(19) United States
(12) Patent Application Publication
(10) Pub. No.: US 2025/0353924 A1
(43) Pub. Date: Nov. 20, 2025

(54) ANTI-CD84 ANTIBODIES AND USES THEREOF

(71) Applicant: CITY OF HOPE, Duarte, CA (US)

(72) Inventors: John C. WILLIAMS, Duarte, CA (US); Alfur HUNG, Duarte, CA (US); Miso PARK, Duarte, CA (US); Flavia PICHIORRI, Duarte, CA (US); Yinghui ZHU, Duarte, CA (US)

(21) Appl. No.: 18/866,493

(22) PCT Filed: May 16, 2023
(86) PCT No.: PCT/US2023/067081

§ 371 Date: Nov. 15, 2024

Related U.S. Application Data
(60) Provisional application No. 63/342,521, filed on May 16, 2022.

(51) Int. Cl. C07K 16/28; A61K 39/00; A61K 47/68; A61P 35/02

(52) U.S. Cl. CPC C07K 16/2896; A61K 47/6849; A61P 35/02; A61K 2039/505; C07K 2317/24; C07K 2317/55; C07K 2317/565; C07K 2317/567; C07K 2317/622; C07K 2317/732

(57) ABSTRACT

Provided herein are, inter alia, antibodies (e.g. humanized antibodies, chimeric antibodies, monoclonal antibodies, antibody fragments (e.g. scFvs)), which bind Cluster of Differentiation 84 (CD84) with high efficiency and specificity. The antibodies provided herein include novel light and heavy chain domain CDRs and framework regions and are, inter alia, useful for treating cancer and other CD84-related diseases.

### CROSS-REFERENCE TO RELATED APPLICATIONS

This International Application claims the benefit of priority under 35 U.S.C. § 119 (e) of the U.S. Patent Application No. 63/342,521, filed on May 16, 2022, which is hereby incorporated by reference in its entirety and for all purposes.

### REFERENCE TO A SEQUENCE LISTING

The Sequence Listing written in file 048440-750001US_SL_ST26.xml, created May 14, 2023, 102, 132 bytes, machine format IBM-PC, MS-Windows operating system, is hereby incorporated by reference in its entirety.

### BACKGROUND

There is a need in the art for anti-CD84 antibodies to act on the tumor microenvironment, resulting in a favorable immune response and subsequent tumor eradication. Provided herein are, inter alia, compositions and methods addressing these and other needs in the art.

### BRIEF SUMMARY OF THE INVENTION

In an aspect, there is provided an anti-CD84 antibody including a light chain variable domain and a heavy chain variable domain, wherein the heavy chain variable domain includes a CDR H1 as set forth in SEQ ID NO:75, a CDR H2 as set forth in SEQ ID NO:76, and a CDR H3 as set forth in SEQ ID NO:77; and wherein the light chain variable domain includes a CDR L1 as set forth in SEQ ID NO:78, a CDR L2 as set forth in SEQ ID NO:79 and a CDR L3 as set forth in SEQ ID NO:80.

In another aspect, there is provided an anti-CD84 antibody including a light chain variable domain and a heavy chain variable domain, wherein the heavy chain variable domain includes a CDR H1 as set forth in SEQ ID NO: 19, a CDR H2 as set forth in SEQ ID NO:20, and a CDR H3 as set forth in SEQ ID NO:21; and wherein the light chain variable domain includes a CDR L1 as set forth in SEQ ID NO:22, a CDR L2 as set forth in SEQ ID NO:23 and a CDR L3 as set forth in SEQ ID NO:24.

In another aspect, there is provided an anti-CD84 antibody including a light chain variable domain and a heavy chain variable domain, wherein the heavy chain variable domain includes a CDR H1 as set forth in SEQ ID NO:39, a CDR H2 as set forth in SEQ ID NO:40, and a CDR H3 as set forth in SEQ ID NO:41; and wherein the light chain variable domain includes a CDR L1 as set forth in SEQ ID NO:42, a CDR L2 as set forth in SEQ ID NO:43 and a CDR L3 as set forth in SEQ ID NO:44.

In another aspect, there is provided an anti-CD84 antibody including a light chain variable domain and a heavy chain variable domain, wherein the heavy chain variable domain includes a CDR H1 as set forth in SEQ ID NO:57, a CDR H2 as set forth in SEQ ID NO:58, and a CDR H3 as set forth in SEQ ID NO:59; and wherein the light chain variable domain includes a CDR L1 as set forth in SEQ ID NO:60, a CDR L2 as set forth in SEQ ID NO:61 and a CDR L3 as set forth in SEQ ID NO:62.

In another aspect, there is provided an anti-CD84 antibody including a light chain variable domain and a heavy chain variable domain, wherein the heavy chain variable domain includes a CDR H1 as set forth in SEQ ID NO:93, a CDR H2 as set forth in SEQ ID NO:94, and a CDR H3 as set forth in SEQ ID NO:95; and wherein the light chain variable domain includes a CDR L1 as set forth in SEQ ID NO:96, a CDR L2 as set forth in SEQ ID NO:97 and a CDR L3 as set forth in SEQ ID NO:98.

In another aspect, there is provided an anti-CD84 antibody, wherein the anti-CD84 antibody binds the same epitope as an antibody including: a heavy chain variable domain including a CDR H1 as set forth in SEQ ID NO:19, a CDR H2 as set forth in SEQ ID NO:20, and a CDR H3 as set forth in SEQ ID NO:21, and a light chain variable domain including a CDR L1 as set forth in SEQ ID NO:22, a CDR L2 as set forth in SEQ ID NO:23 and a CDR L3 as set forth in SEQ ID NO:24.

In another aspect, there is provided an anti-CD84 antibody, wherein the anti-CD84 antibody binds the same epitope as an antibody including: a heavy chain variable domain including a CDR H1 as set forth in SEQ ID NO:39, a CDR H2 as set forth in SEQ ID NO:40, and a CDR H3 as set forth in SEQ ID NO:41, and a light chain variable domain including a CDR L1 as set forth in SEQ ID NO:42, a CDR L2 as set forth in SEQ ID NO:43 and a CDR L3 as set forth in SEQ ID NO:44.

In another aspect, there is provided an anti-CD84 antibody, wherein the anti-CD84 antibody binds the same epitope as an antibody including: a heavy chain variable domain including a CDR H1 as set forth in SEQ ID NO:57, a CDR H2 as set forth in SEQ ID NO:58, and a CDR H3 as set forth in SEQ ID NO:59, and a light chain variable domain including a CDR L1 as set forth in SEQ ID NO:60, a CDR L2 as set forth in SEQ ID NO:61 and a CDR L3 as set forth in SEQ ID NO:62.

In another aspect, there is provided an anti-CD84 antibody, wherein the anti-CD84 antibody binds the same epitope as an antibody including: a heavy chain variable domain including a CDR H1 as set forth in SEQ ID NO:75, a CDR H2 as set forth in SEQ ID NO:76, and a CDR H3 as set forth in SEQ ID NO:77, and a light chain variable domain including a CDR L1 as set forth in SEQ ID NO:78, a CDR L2 as set forth in SEQ ID NO:79 and a CDR L3 as set forth in SEQ ID NO:80.

In another aspect, there is provided an anti-CD84 antibody, wherein the anti-CD84 antibody binds the same epitope as an antibody including: a heavy chain variable domain including a CDR H1 as set forth in SEQ ID NO:93, a CDR H2 as set forth in SEQ ID NO:94, and a CDR H3 as set forth in SEQ ID NO:95, and a light chain variable domain including a CDR L1 as set forth in SEQ ID NO:96, a CDR L2 as set forth in SEQ ID NO:97 and a CDR L3 as set forth in SEQ ID NO:98.

In another aspect, there is provided an isolated nucleic acid encoding an anti-CD84 antibody as provided herein including embodiments thereof.

In another aspect, there is provided a cell including an anti-CD84 antibody as provided herein including embodiments thereof, or a nucleic acid as provided here in including embodiments thereof.

In another aspect, there is provided a pharmaceutical composition including a therapeutically effective amount of an antibody as provided herein including embodiments thereof and a pharmaceutically acceptable excipient.

In another aspect, there is provided a method of forming an antibody capable of binding to CD84, the method including immunizing a mammal with a peptide comprising the sequence of SEQ ID NO:111.

In another aspect, there is provided a method of treating cancer in a subject in need thereof, said method includes administering to a subject a therapeutically effective amount of an anti-CD84 antibody as provided herein including embodiments thereof or a pharmaceutical composition as provided herein including embodiments thereof, thereby treating cancer in said subject.

### BRIEF DESCRIPTION OF THE DRAWINGS

FIG. 1. illustrates recombinant CD84 protein constructs where the extracellular domain (ECD) of human CD84 is fused with either mouse lgG2a.Fc (left) or human IgA1.Fc (right).

FIG. 2 illustrates the purification and characterization of the recombinant CD84 proteins fused to either IgG2a (top) or IgA1 (bottom).

FIG. 3 illustrates exemplary flow cytometry data showing that 16 CD84 clones were capable of binding human CD84 on the cell surface of a mouse myeloma cell line (ECACC).

FIG. 4 illustrates exemplary data from flow cytometry experiments testing the ability of six CD84 clones (2H6, 4B11, 5C6, 7D2, 8A5, and 13D11) to bind human myeloma cell line HL-60, T cell line Jurkat, lymphoma cell line K-562, and mammary gland cell line SKBR3 (ATCC).

FIG. 5 illustrates exemplary data from ELISA experiments showing that the 2H6, 4B11, 5C6, 7D2, 8A5, and 13D11 clones were of Igγ1 and Igk subtypes.

FIGS. 6A-6E show purification and characterization of the chimeric Fab for clones 5C6 (FIG. 6A), 7D2 (FIG. 6B), 8A5 (FIG. 6C), 13D11 (FIG. 6E). FIG. 6A is a chromatogram depicting purification of chimeric Fab of clone 5C6 using size exclusion chromatography. FIG. 6B is a chromatogram depicting purification of chimeric Fab of clone 7D2 using size exclusion chromatography. FIG. 6C is a chromatogram depicting purification of chimeric Fab of clone 8A5 using size exclusion chromatography. FIG. 6D shows SDS-PAGE gels with reducing (R) and non-reducing (NR) conditions characterizing the purified chimeric Fab of clones 5C6, 8A5 and 7D2. FIG. 6E is a chromatogram depicting purification of chimeric Fab of clone 7D2 using size exclusion chromatography (left) and a SDS-PAGE gel with reducing (R) and non-reducing (NR) conditions characterizing the purified chimeric Fab of clone 13D11.

FIGS. 7A-7D show sensorgrams from SPR experiments depicting binding of chimeric anti-CD84 Fabs for clones 13D11 (FIG. 7A), 5C6 (FIG. 7B), 7D2 (FIG. 7C), and 8A5 (FIG. 7D). Anti-HIS antibodies were immobilized on a SPR biosensor chip and anti-CD84 chimeric Fabs were injected at concentrations of 300 nM, 100 nM, 30 nM, and 10 nM at 25° C.

FIG. 8 illustrates thermal stability data and melting point temperatures (Tm) of chimeric anti-CD84 Fabs for 5C6, 7D2, 8A5 and 13D11 clones.

FIG. 9 depicts ribbon diagrams of the atomic structures of the murine chimeric anti-CD84 8A5, 7D2, and 5C6 clones derived from X-ray diffraction data.

FIGS. 10A-10F show purification and characterization of the humanized anti-CD84 Fabs and antibodies for 4B11 and 13D11 clones. FIG. 10A is a chromatogram depicting purification of the humanized anti-CD84 antibody 4B11 clone using size exclusion chromatography. FIG. 10B is a chromatogram depicting purification of the humanized anti-CD84 antibody 13D11 clone using size exclusion chromatography. FIG. 10C shows a SDS-PAGE gel with reducing (R) and non-reducing (NR) conditions characterizing the purified humanized anti-CD84 antibody 4B11 and 13D11 clones. FIG. 10D is a chromatogram depicting purification of the humanized anti-CD84 4B11 clone Fab using size exclusion chromatography. FIG. 10E is a chromatogram depicting purification of the humanized anti-CD84 13D11 clone Fab using size exclusion chromatography. FIG. 10F shows a SDS-PAGE gel with reducing (R) and non-reducing (NR) conditions characterizing the purified humanized anti-CD84 Fabs of clones 4B11 and 13D11.

FIGS. 11A-11B illustrates CD84 expression in specimens from patients with AML compared to healthy donors. FIG. 11A is a scatter plot showing the CD84 messenger RNA (mRNA) expression in GSE9476 and GSE13159 data sets including specimens from patient with AML vs health donor highlighted increased CD84 expression in AML specimens, especially in the myelomonocytic and monoblastic leukemia patients with t (11q23) and inv (16) abnormalities; GSE9476 (health donor n=38; AML n=26); GSE13159 (health donor n=73; AML n=541). FIG. 11B is a violin plot showing CD84 surface protein expression in healthy donor (n=4), AML patient specimens (n=4) and AML cell lines (n=6) analyzed by flow cytometry highlighted CD84 is highly expressed in AML primary patient cells and AML cell lines, relative to health donor. Y axis indicated Mean Fluorescent Intensity (MFI) of CD84 relative to isotype. FIG. 11C is survival curve showing highly CD84 expression is associated with shorter overall survival (OS) in AML patients (GSE10358). * p<0.05, ** p<0.01, *** p<0.001, **** p<0.0001

FIGS. 12A-12C shows exemplary data from experiments using short hairpin RNA (sh) to knockdown CD84 in AML cell lines (FIG. 12A), primary patient AML cells (FIG. 12B), and the THP 1-xenograft mouse model (FIG. 12C). FIG. 12A (left panel) is a line graph representing cell proliferative analysis of THP1 and HEL cells transduced with shControl (shCtr1) or shCD84 lentiviral vector indicated CD84 knockdown inhibit cell proliferation in AML cell lines. Error bars represent SEM from three independent experiments. FIG. 12A (right panel) is a bar graph showing apoptosis analysis indicated Annexin-APC/DAPI in THP1 and HEL cells transduced with shCtr1 or shCD84 lentiviral vector indicated CD84 knockdown induced AML cell apoptosis. FIG. 12B is a bar chart showing apoptosis analysis indicated by Annexin-APC/DAPI in two AML patient specimens transduced with shCtr1 or shCD84 lentiviral vector demonstrated that CD84 knockdown induced cell apoptosis in AML primary patient cells. Error bars represent SEM from three technological experiments. FIG. 12C illustrates bioluminescent imaging showing the tumor burden in xenograft NSG mice on day 35 following shCtr1 or shCD84 transduced THP1-luciferase cell transplantation. Kaplan-Meier analysis of survival of THP1-luciferase cell (shCtr1 or shCD84) transplanted NSG mice (n=5 per group). Attenuated tumor burden as well as prolonged survival were observed in the mice transplanted with CD84 knockdown cells, relative to control mice. * p<0.05, ** p<0.01, *** p<0.001.

FIGS. 13A-13E illustrates exemplary data from experiments showing that CD84 knockdown inhibited MLL-AF9 cell growth in vitro and attenuated cell engraftment in vivo. FIG. 13A showing workflow of generating the MLL-AF9 AML cells with CD84 knockdown. Briefly, c-kit positive bone marrow cells collected from C57BL/6 mice (CD45.2) were transduced with MLL-AF9 retrovirus, and then transduced with shCtr1, shCD84-1 or shCD84-2 lentivirus. The cells were used for cell proliferative analysis, colony formation assay. MLL-AF9 transduced cells were also transplanted into irradiated recipient cells for leukemogenesis. FIG. 13B is a line graph representing cell proliferative analysis of MLL-AF9 cells transduced with shCtr1 or shCD84 lentiviral vector indicated CD84 knockdown inhibit cell proliferation in MLL-AF9 AML cells. FIG. 13C shown the graph and bar chart of MLL-AF9 AML cells which transduced with shCtr1, shCD84-1 or shCD84-2 and then plated for colony formation assay. These results indicated the CD84 knockdown inhibit AML cell colony forming capacity. FIG. 13D illustrated exemplary data from mice experiment showing that CD84 knockdown inhibits AML engraftment in MLL-AF9 xenograft mouse model. Scatter plot showing donor AML mouse cell engraftment (mouse CD45.2) in bone marrow. FIG. 13E showing MLL-AF9 xenograft with CD84 knockdown cells decrease splenomegaly. * p<0.05, ** p<0.01, *** p<0.001, **** p<0.0001.

FIGS. 14A-14B illustrates exemplary data from experiments showing that CD84 knockdown decreased AML engraftment in AML Patient Derived Xenograft (PDX) mouse model. Scatter plot showing lower AML engraftment (human CD45/CD33 positive ratio) in bone marrow, spleen, peripheral blood (PB) (FIG. 14A) as well as reduced spleen weight (FIG. 14B) of NSG mice transplanted with AML primary cells transfected with shCD84 lentivirus, relative to shCtr1 groups. * p<0.05, ** p<0.01.

FIGS. 15A-15B illustrates exemplary data from experiments in which AML cells cultured with total PBMC or NK cells from a healthy donor were treated with anti-CD84 antibodies. FIG. 15A shows offset histograms demonstrating CD84 expression detected by flow cytometry in CD84 high expressed cell THP1 and CD84 low/null expressed cell MMIS (top panel) and bar charts showing the anti-CD84 monoclonal antibody clones (5C6, 7D2 and 8A5) induced antibody-dependent cellular cytotoxicity (ADCC) in THP1 (middle panel) and MMIS cells (bottom panel), highlighted that anti-CD84 7D2 antibody shown high anti-leukemic activity and specificity. FIG. 15B shows line graphs illustrating that anti-CD84 7D2 antibody induced ADCC of THP1-luciferase cells (Target cells) in presence of total PBMC (top and middle panels) or NK cells (bottom panel) as Effector cells, as indicated by 7-ADD positive ratio. The results highlighted that anti-CD84 antibody induced ADCC were E: T ratio-dependent and antibody dose-dependent. Health donor PBMC (n=2); Health donor NK cell (n=2).

FIGS. 16A-16B illustrates exemplary data from experiments showing that anti-CD84 antibody treatment (clone 7D2) decreases tumor burden and extends survival in the THP1 and U937-xenograft model. FIG. 16A shown bioluminescent imaging demonstrated the tumor burden in luciferase expressed THP1-xenograft NSG mice following anti-human IgG/anti-CD84 antibody treatment (clone 7D2). Kaplan-Meier analysis of survival of treated NSG mice (n=6 per group). Attenuated tumor burden as well as prolonged survival highlighted that anti-CD84 antibody (clone 7D2) shown significantly anti-leukemic effect. FIG. 16B shown bioluminescent imaging indicated the tumor burden in luciferase expressed U937-xenograft NSG mice following anti-human IgG/anti-CD84 antibody treatment (clone 7D2). Kaplan-Meier analysis of survival of treated NSG mice (n=6 for IgG group; n=7 for anti-CD84 7D2 antibody group). Attenuated tumor burden as well as prolonged survival highlighted that anti-CD84 antibody (clone 7D2) shown significantly anti-leukemic effect. ** p<0.01, *** p<0.001.

FIG. 17 illustrates exemplary data from experiments showing that anti-CD84 antibody treatment (clone 7D2) decreases AML engraftment in AML Patient Derived Xenograft (PDX) mouse model. Scatter plot showing AML engraftment (Human CD45/CD33 positive ratio) in bone marrow, spleen, peripheral blood as well as spleen weight of AML PDX transplanted NSG mice treated with anti-human IgG or anti-CD84 antibody (clone 7D2) (n=6 per group). The results indicated that anti-CD84 antibody (clone 7D2) significantly inhibited AML progression in vivo. Error bars represent SEM. ** p<0.01, *** p<0.001.

FIG. 18 shows statistics from data collection and refinement of an 8A5-CD84 complex.

FIG. 19 shows the crystal structure of an 8A5-CD84 complex using X-ray crystallography. Note, the dimer interface is generated by symmetry elements of the crystal.

FIGS. 20A-20F show data of co-elution of anti-CD84 Fabs binding using fast protein liquid chromatography (FPLC) data. FIGS. 20A-20C show chromatograms of CD84 mixed with the individual Fabs. The shift to earlier volumes indicates the formation of a Fab/CD84 complex. FIGS. 20D-20F show chromatograms using a mixture of two Fabs and the CD84. Note the X axes are different-FIG. 20D is from 10 to 15 ml and FIGS. 20E-20F are 8 to 15 mL. In FIG. 20D, the shift of the complex is similar to the individual Fab traces (˜11 ml), indicating that 7D2 and 5C6 bind to an epitope that is either shared or in proximity such that one sterically occludes the other from binding to CD84. In FIGS. 20E-20F, there is a substantial shift to the left (early elution times, ˜9.5 ml) indicating that the bind to distinct, non-overlapping epitopes.

### DETAILED DESCRIPTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., EGFR) in other proteins with different numbering systems. For example, by performing a simple sequence alignment with a protein (e.g., EGFR) the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the residue to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
   (see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2: 482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P (N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

“CD84” as referred to herein includes any of the recombinant or naturally-occurring forms of the Cluster of Differentiation 84 (CD84) proteins or variants or homologs thereof that comprise the CD84 complex that mediates signal transduction and maintains CD84 complex activity (e.g. within at least 50%, 80%, 90%, 95%, 96%, 97%, 98%, 99% or 100% activity compared to the CD84 complex). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD84 proteins in the CD84 complex. In embodiments, the CD84 protein is substantially identical to the protein identified by the UniProt reference number Q9UIB8 or a variant or homolog having substantial identity thereto.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab₂, bispecific Fab₂, trispecific Fab₃, monovalent IgGs, scFv, bispecific antibodies, bispecific diabodies, trispecific triabodies, scFv-Fc, minibodies, IgNAR, V-NAR, hclgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable heavy chain provided herein includes in N-terminal to C-terminal direction a CDR H1, a CDR H2 and a CDR H3. In embodiments, the CDRs of the light chain are referred to as CDR1, CDR2, and CDR3 of VL and the CDRs of the heavy chain are referred to as CDR1, CDR2, and CDR3 of VH. See, for example the tables as provided herein.

The terms “FR L1”, “FR L2”, “FR L3” and “FR L4” as provided herein are used according to their common meaning in the art and refer to the framework regions (FR) 1, 2, 3 and 4 of the variable light (L) chain of an antibody. In embodiments, the variable light chain provided herein includes in N-terminal to C-terminal direction a FR L1, a FR L2, a FR L3 and a FR L4. Likewise, the terms “FR H1”, “FR H2”, “FR H3” and “FR H4” as provided herein are used according to their common meaning in the art and refer to the framework regions (FR) 1, 2, 3 and 4 of the variable heavy (H) chain of an antibody. In embodiments, the variable heavy chain provided herein includes in N-terminal to C-terminal direction a FR H1, a FR H2, a FR H3 and a FR H4.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins. The term “light chain” is used according to its ordinary meaning in the biological arts, and refers to the polypeptide formed by a light chain variable domain (VL) and a light chain constant domain (CL). Likewise, the term “heavy chain” is used according to its ordinary meaning in the biological arts, and refers to the polypeptide formed by a heavy chain variable domain (VH) and one or more heavy chain constant domains (CH1, CH2, CH3).

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F (ab)′2, a dimer of Fab which itself is a light chain joined to V_{H}-C_{H} by a disulfide bond. The F (ab)′2 may be reduced under mild conditions to break the disulfide linkage in the hinge region, thereby converting the F (ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). The term “antibody” as referred to herein further includes antibody variants such as single domain antibodies. Thus, in embodiments an antibody includes a single monomeric variable antibody domain. Thus, in embodiments, the antibody, includes a variable light chain (VL) domain or a variable heavy chain (VH) domain. In embodiments, the antibody is a variable light chain (VL) domain or a variable heavy chain (VH) domain.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A single-chain variable fragment (scFv) is typically a fusion protein of the variable domains of the heavy (VH) and light chain (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

The term “antigen” as provided herein refers to molecules capable of binding to the antibody binding domain provided herein. An “antigen binding domain” as provided herein is a region of an antibody that binds to an antigen (epitope). As described above, the antigen binding domain is generally composed of one constant and one variable domain of each of the heavy and the light chain (VL, VH, CL and CH1, respectively). The paratope or antigen-binding site is formed on the N-terminus of the antigen binding domain. The two variable domains of an antigen binding domain typically bind the epitope on an antigen.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31 (3): 169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (e.g, variable region including domain VH and VL) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “ligand” refers to an agent, e.g., a polypeptide or other molecule, capable of binding to a receptor or antibody, antibody variant, antibody region or fragment thereof. In embodiments, a ligand is an antigen, fragment or portion thereof.

Techniques for conjugating therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies'84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)). As used herein, the term “antibody-drug conjugate” or “ADC” refers to a therapeutic agent conjugated or otherwise covalently bound to to an antibody.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “EGFR protein” or “EGFR” as used herein includes any of the recombinant or naturally-occurring forms of epidermal growth factor receptor, also known as Proto-oncogene c-ErbB-1, Receptor tyrosine-protein kinase erbB-1, ERBB, ERBB1, HER1, or variants or homologs thereof that maintain EGFR activity (e.g. within at least 50%, 80%, 90%, 95%, 96%, 97%, 98%, 99% or 100% activity compared to EGFR). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein. In embodiments, the EGFR protein is substantially identical to the protein identified by the UniProt reference number P00533 or a variant or homolog having substantial identity thereto.

Epidermal growth factor receptor, also known as EGFR, ErbB1 and HER1, is a cell-surface receptor of the epidermal growth factor family of extracellular ligands. Alterations in EGFR activity have been implicated in certain cancers. In embodiments, a gene encoding an EGFR polypeptide is provided that is formed by removal of nucleic acid sequences that encode polypeptides including the membrane distal EGF-binding domain and the cytoplasmic signaling tail (a “truncated EGFR”, “tEGFR” or “EGFRt”), but retains the extracellular domain IV epitope recognized by any anti-EGFR antibody (e.g., anti-domain IV EGFR antibody) provided herein including embodiments thereof. In embodiments, tEGFR does not include EGFR domain III.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetofection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

When the label or detectable moiety is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the PSMA antibody or functional antibody fragment by a group, which enables the formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as 223Ra for RAIT may be used. In certain embodiments, chelating moieties may be used to attach a PET imaging agent, such as an Al-¹⁸F complex, to a targeting molecule for use in PET analysis.

“Contacting” is used in accordance with its plain ordinary meaning and refers to the process of allowing at least two distinct species (e.g. antibodies and antigens) to become sufficiently proximal to react, interact, or physically touch. It should be appreciated; however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with the compounds or methods provided herein. The disease may be a cancer. The cancer may refer to a solid tumor malignancy. Solid tumor malignancies include malignant tumors that may be devoid of fluids or cysts. For example, the solid tumor malignancy may include breast cancer, ovarian cancer, pancreatic cancer, cervical cancer, gastric cancer, renal cancer, head and neck cancer, bone cancer, skin cancer or prostate cancer. In some further instances, “cancer” refers to human cancers and carcinomas, sarcomas, adenocarcinomas, lymphomas, leukemias, including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including acute myeloid leukemia (AML), ALL, and CML), or multiple myeloma.

As used herein, the term “cancer” refers to all types of cancer, neoplasm or malignant tumors found in mammals (e.g., humans), including leukemia, carcinomas and sarcomas. Exemplary cancers that may be treated with a compound or method provided herein include breast cancer, colon cancer, kidney cancer, leukemia, lung cancer, melanoma, ovarian cancer,

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g. a protein associated disease, a cancer (e.g., breast cancer, lung cancer)) means that the disease (e.g. cancer) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

A “therapeutic agent” as referred to herein, is a composition useful in treating or preventing a disease such as cancer (e.g., leukemia). In embodiments, the therapeutic agent is an anti-cancer agent. “Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with a compound, pharmaceutical composition, or method provided herein. In embodiments, the disease is cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma (Mantel cell lymphoma), head and neck cancer, colorectal cancer, prostate cancer, pancreatic cancer, melanoma, breast cancer, neuroblastoma).

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma (e.g., Mantel cell lymphoma, follicular lymphoma, diffuse large B-cell lymphoma, marginal zona lymphoma, Burkitt's lymphoma), sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia (e.g., lymphoblastic leukemia, chronic lymphocytic leukemia, hairy cell leukemia), acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The P388 leukemia model is widely accepted as being predictive of in vivo anti-leukemic activity. It is believed that a compound that tests positive in the P388 assay will generally exhibit some level of anti-leukemic activity in vivo regardless of the type of leukemia being treated. Accordingly, the present application includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “associated” or “associated with” in the context of a substance or substance activity or function associated with a disease (e.g., cancer) means that the disease is caused by (in whole or in part), or a symptom of the disease is caused by (in whole or in part) the substance or substance activity or function.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. For example, certain methods herein treat cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma). For example, certain methods herein treat cancer by decreasing or reducing or preventing the occurrence, growth, metastasis, or progression of cancer; or treat cancer by decreasing a symptom of cancer. Symptoms of cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma) would be known or may be determined by a person of ordinary skill in the art.

As used herein the terms “treatment,” “treat,” or “treating” refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

An “effective amount” is an amount sufficient to accomplish a stated purpose (e.g. achieve the effect for which it is administered, treat a disease, reduce enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein relative to the absence of the antagonist. A “function disrupting amount,” as used herein, refers to the amount of antagonist required to disrupt the function of an enzyme or protein relative to the absence of the antagonist. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

In embodiments, the methods provided herein further include administering to the subject an additional therapeutic agent. As described above, a therapeutic agent is a composition useful in treating or preventing a disease such as cancer. In embodiments, the additional therapeutic agent is an anti-cancer agent.

The terms “anti-cancer agent” and “anticancer agent” are used in accordance with their plain ordinary meaning and refer to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec.®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin Il (including recombinant interleukin II, or rlL.sub.2), interferon alfa-2a; interferon alfa-2b; interferon alfa-n1; interferon alfa-n3; interferon beta-la; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol.™ (i.e. paclitaxel), Taxotere.™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin Al (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, Inanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to 111 In, 90Y, or 131I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like, that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Anti-Cd84 Antibodies

Provided herein are, inter alia, antibodies capable of binding Cluster of Differentiation 84 (CD84). The antibodies provided herein include novel light chain and heavy chain sequences and bind CD84 with high efficiency and specificity. Based on these binding features the antibodies provided herein are able to effectively convey antibody-dependent cellular cytotoxicity (ADCC) in a cancer microenvironment, which results in cell-mediated immune defense, whereby an effector cell of the immune system actively lyses a CD84-expressing cancer cell. Thus, the CD84 antibodies as provided herein including embodiments thereof, are useful for anti-cancer therapeutic purposes.

In an aspect, there is provided an anti-CD84 antibody including a light chain variable domain and a heavy chain variable domain, wherein the heavy chain variable domain includes a CDR H1 as set forth in SEQ ID NO:75, a CDR H2 as set forth in SEQ ID NO:76, and a CDR H3 as set forth in SEQ ID NO:77; and wherein the light chain variable domain includes a CDR L1 as set forth in SEQ ID NO:78, a CDR L2 as set forth in SEQ ID NO:79 and a CDR L3 as set forth in SEQ ID NO:80.

In another aspect, there is provided an anti-CD84 antibody including a light chain variable domain and a heavy chain variable domain, wherein the heavy chain variable domain includes a CDR H1 including the sequence as set forth in SEQ ID NO:75, a CDR H2 including the sequence as set forth in SEQ ID NO:76, and a CDR H3 including the sequence as set forth in SEQ ID NO: 77; and wherein the light chain variable domain includes a CDR L1 including the sequence as set forth in SEQ ID NO:78, a CDR L2 including the sequence as set forth in SEQ ID NO:79 and a CDR L3 including the sequence as set forth in SEQ ID NO:80.

In embodiments, the anti-CD84 antibody is a chimeric antibody. In embodiments, the anti-CD84 antibody is a humanized antibody.

In embodiments, the heavy chain variable domain includes a FR H1 as set forth in SEQ ID NO: 81, a FR H2 as set forth in SEQ ID NO:82, a FR H3 as set forth in SEQ ID NO:83 and a FR H4 as set forth in SEQ ID NO:84. In embodiments, the light chain variable domain includes a FR L1 as set forth in SEQ ID NO:85, a FR L2 as set forth in SEQ ID NO:86, a FR L3 as set forth in SEQ ID NO:87 and a FR L4 as set forth in SEQ ID NO:88.

In embodiments, the heavy chain variable domain includes a FR H1 including the sequence as set forth in SEQ ID NO:81, a FR H2 including the sequence as set forth in SEQ ID NO: 82, a FR H3 including the sequence as set forth in SEQ ID NO:83 and a FR H4 including the sequence as set forth in SEQ ID NO:84. In embodiments, the light chain variable domain includes a FR L1 including the sequence as set forth in SEQ ID NO:85, a FR L2 including the sequence as set forth in SEQ ID NO:86, a FR L3 including the sequence as set forth in SEQ ID NO:87 and a FR L4 including the sequence as set forth in SEQ ID NO:88.

In embodiments, the heavy chain variable domain includes the sequence of SEQ ID NO: 89. In embodiments, the heavy chain variable domain has the sequence of SEQ ID NO:89. In embodiments, the light chain variable domain includes the sequence of SEQ ID NO:90. In embodiments, the light chain variable domain has the sequence of SEQ ID NO:90.

In embodiments, the anti-CD84 antibody includes a heavy chain including the sequence of SEQ ID NO:91. In embodiments, the anti-CD84 antibody includes a heavy chain that has the sequence of SEQ ID NO:91. In embodiments, the anti-CD84 antibody includes a light chain including the sequence of SEQ ID NO:92. In embodiments, the anti-CD84 antibody includes a light chain that has the sequence of SEQ ID NO:92. In embodiments, the anti-CD84 antibody is referred to herein as clone 7D2.

In another aspect, there is provided an anti-CD84 antibody including a light chain variable domain and a heavy chain variable domain, wherein the heavy chain variable domain includes a CDR H1 as set forth in SEQ ID NO:19, a CDR H2 as set forth in SEQ ID NO:20, and a CDR H3 as set forth in SEQ ID NO:21; and wherein the light chain variable domain includes a CDR L1 as set forth in SEQ ID NO:22, a CDR L2 as set forth in SEQ ID NO:23 and a CDR L3 as set forth in SEQ ID NO:24.

In another aspect, there is provided an anti-CD84 antibody including a light chain variable domain and a heavy chain variable domain, wherein the heavy chain variable domain includes a CDR H1 including the sequence as set forth in SEQ ID NO:19, a CDR H2 including the sequence as set forth in SEQ ID NO:20, and a CDR H3 including the sequence as set forth in SEQ ID NO: 21; and wherein the light chain variable domain includes a CDR L1 including the sequence as set forth in SEQ ID NO:22, a CDR L including the sequence as set forth in SEQ ID NO:23 and a CDR L3 including the sequence as set forth in SEQ ID NO:24.

In embodiments, the anti-CD84 antibody is a humanized antibody. In embodiments, the antibody includes a heavy chain including the sequence of SEQ ID NO:37. In embodiments, the antibody includes a heavy chain that has the sequence of SEQ ID NO:37. In embodiments, the antibody includes a light chain including the sequence of SEQ ID NO:38. In embodiments, the antibody includes a light chain that has the sequence of SEQ ID NO:38.

In embodiments, the anti-CD84 antibody is a chimeric antibody. In embodiments, the heavy chain variable domain includes a FR H1 as set forth in SEQ ID NO:25, a FR H2 as set forth in SEQ ID NO:26, a FR H3 as set forth in SEQ ID NO:27 and a FR H4 as set forth in SEQ ID NO: 28. In embodiments, the light chain variable domain includes a FR L1 as set forth in SEQ ID NO: 29, a FR L2 as set forth in SEQ ID NO:30, a FR L3 as set forth in SEQ ID NO:31 and a FR L4 as set forth in SEQ ID NO:32. In embodiments, the heavy chain variable domain includes a FR H1 including the sequence as set forth in SEQ ID NO:25, a FR H2 including the sequence as set forth in SEQ ID NO:26, a FR H3 including the sequence as set forth in SEQ ID NO:27 and a FR H4 including the sequence as set forth in SEQ ID NO:28. In embodiments, the light chain variable domain includes a FR L1 including the sequence as set forth in SEQ ID NO:29, a FR L2 including the sequence as set forth in SEQ ID NO:30, a FR L3 including the sequence as set forth in SEQ ID NO: 31 and a FR L4 including the sequence as set forth in SEQ ID NO:32.

In embodiments, the heavy chain variable domain includes the sequence of SEQ ID NO: 33. In embodiments, the heavy chain variable domain has the sequence of SEQ ID NO:33. In embodiments, the light chain variable domain includes the sequence of SEQ ID NO:34. In embodiments, the light chain variable domain has the sequence of SEQ ID NO:34. In embodiments, the antibody includes a heavy chain including the sequence of SEQ ID NO:35. In embodiments, the antibody includes a heavy chain that has the sequence of SEQ ID NO:35. In embodiments, the antibody includes a light chain including the sequence of SEQ ID NO:36. In embodiments, the antibody includes a light chain that has the sequence of SEQ ID NO:36. In embodiments, the anti-CD84 antibody is referred to herein as clone 13D11.

In another aspect, there is provided an anti-CD84 antibody including a light chain variable domain and a heavy chain variable domain, wherein the heavy chain variable domain includes a CDR H1 as set forth in SEQ ID NO:39, a CDR H2 as set forth in SEQ ID NO:40, and a CDR H3 as set forth in SEQ ID NO:41; and wherein the light chain variable domain includes a CDR L1 as set forth in SEQ ID NO:42, a CDR L2 as set forth in SEQ ID NO:43 and a CDR L3 as set forth in SEQ ID NO:44.

In another aspect, there is provided an anti-CD84 antibody including a light chain variable domain and a heavy chain variable domain, wherein the heavy chain variable domain includes a CDR H1 including the sequence as set forth in SEQ ID NO:39, a CDR H2 including the sequence as set forth in SEQ ID NO:40, and a CDR H3 including the sequence as set forth in SEQ ID NO: 41; and wherein the light chain variable domain includes a CDR L1 including the sequence as set forth in SEQ ID NO:42, a CDR L2 including the sequence as set forth in SEQ ID NO:43 and a CDR L3 including the sequence as set forth in SEQ ID NO:44.

In embodiments, the anti-CD84 antibody is a chimeric antibody. In embodiments, the anti-CD84 antibody is a humanized antibody. In embodiments, heavy chain variable domain includes a FR H1 as set forth in SEQ ID NO:45, a FR H2 as set forth in SEQ ID NO:46, a FR H3 as set forth in SEQ ID NO:47 and a FR H4 as set forth in SEQ ID NO:48. In embodiments, the light chain variable domain includes a FR L1 as set forth in SEQ ID NO:49, a FR L2 as set forth in SEQ ID NO:50, a FR L3 as set forth in SEQ ID NO:51 and a FR L4 as set forth in SEQ ID NO: 52. In embodiments, heavy chain variable domain includes a FR H1 including the sequence as set forth in SEQ ID NO:45, a FR H2 including the sequence as set forth in SEQ ID NO:46, a FR H3 including the sequence as set forth in SEQ ID NO:47 and a FR H4 including the sequence as set forth in SEQ ID NO:48. In embodiments, the light chain variable domain includes a FR L1 including the sequence as set forth in SEQ ID NO:49, a FR L2 including the sequence as set forth in SEQ ID NO:50, a FR L3 including the sequence as set forth in SEQ ID NO:51 and a FR L4 including the sequence as set forth in SEQ ID NO:52.

In embodiments, the heavy chain variable domain includes the sequence of SEQ ID NO: 53. In embodiments, the heavy chain variable domain has the sequence of SEQ ID NO:53. In embodiments, the light chain variable domain includes the sequence of SEQ ID NO:54. In embodiments, the light chain variable domain has the sequence of SEQ ID NO:54. In embodiments, the anti-CD84 antibody includes a heavy chain including the sequence of SEQ ID NO: 55. In embodiments, the anti-CD84 antibody includes a heavy chain that has the sequence of SEQ ID NO:55. In embodiments, the anti-CD84 antibody includes a light chain including the sequence of SEQ ID NO:56. In embodiments, the anti-CD84 antibody includes a light chain that has the sequence of SEQ ID NO:56. In embodiments, the anti-CD84 antibody is referred to herein as clone 2H7.

In another aspect, there is provided an anti-CD84 antibody including a light chain variable domain and a heavy chain variable domain, wherein the heavy chain variable domain includes a CDR H1 as set forth in SEQ ID NO:57, a CDR H2 as set forth in SEQ ID NO:58, and a CDR H3 as set forth in SEQ ID NO:59; and wherein the light chain variable domain includes a CDR L1 as set forth in SEQ ID NO:60, a CDR L2 as set forth in SEQ ID NO:61 and a CDR L3 as set forth in SEQ ID NO:62.

In another aspect, there is provided an anti-CD84 antibody including a light chain variable domain and a heavy chain variable domain, wherein the heavy chain variable domain includes a CDR H1 including the sequence as set forth in SEQ ID NO:57, a CDR H2 including the sequence as set forth in SEQ ID NO:58, and a CDR H3 including the sequence as set forth in SEQ ID NO: 59; and wherein the light chain variable domain includes a CDR L1 including the sequence as set forth in SEQ ID NO:60, a CDR L2 including the sequence as set forth in SEQ ID NO:61 and a CDR L3 including the sequence as set forth in SEQ ID NO:62.

In embodiments, the anti-CD84 antibody is a chimeric antibody. In embodiments, the anti-CD84 antibody is a humanized antibody. In embodiments, the heavy chain variable domain includes a FR H1 as set forth in SEQ ID NO:63, a FR H2 as set forth in SEQ ID NO:64, a FR H3 as set forth in SEQ ID NO:65 and a FR H4 as set forth in SEQ ID NO:66. In embodiments, the light chain variable domain includes a FR L1 as set forth in SEQ ID NO:67, a FR L2 as set forth in SEQ ID NO:68, a FR L3 as set forth in SEQ ID NO:69 and a FR L4 as set forth in SEQ ID NO: 70. In embodiments, the heavy chain variable domain includes a FR H1 including the sequence as set forth in SEQ ID NO:63, a FR H2 including the sequence as set forth in SEQ ID NO: 64, a FR H3 including the sequence as set forth in SEQ ID NO:65 and a FR H4 including the sequence as set forth in SEQ ID NO:66. In embodiments, the light chain variable domain includes a FR L1 including the sequence as set forth in SEQ ID NO:67, a FR L2 including the sequence as set forth in SEQ ID NO:68, a FR L3 including the sequence as set forth in SEQ ID NO:69 and a FR L4 including the sequence as set forth in SEQ ID NO:70.

In embodiments, the heavy chain variable domain includes the sequence of SEQ ID NO: 71. In embodiments, the heavy chain variable domain has the sequence of SEQ ID NO:71. In embodiments, the light chain variable domain includes the sequence of SEQ ID NO:72. In embodiments, the light chain variable domain has the sequence of SEQ ID NO:72. In embodiments, the anti-CD84 antibody includes a heavy chain including the sequence of SEQ ID NO: 73. In embodiments, the anti-CD84 antibody includes a heavy chain that has the sequence of SEQ ID NO:73. In embodiments, the anti-CD84 antibody includes a light chain including the sequence of SEQ ID NO:74. In embodiments, the anti-CD84 antibody includes a light chain that has the sequence of SEQ ID NO:74. In embodiments, the anti-CD84 antibody is referred to herein as clone 5C6.

In another aspect, there is provided an anti-CD84 antibody including a light chain variable domain and a heavy chain variable domain, wherein the heavy chain variable domain includes a CDR H1 as set forth in SEQ ID NO:93, a CDR H2 as set forth in SEQ ID NO:94, and a CDR H3 as set forth in SEQ ID NO:95; and wherein the light chain variable domain includes a CDR L1 as set forth in SEQ ID NO:96, a CDR L2 as set forth in SEQ ID NO:97 and a CDR L3 as set forth in SEQ ID NO:98.

In another aspect, there is provided an anti-CD84 antibody including a light chain variable domain and a heavy chain variable domain, wherein the heavy chain variable domain includes a CDR H1 including the sequence as set forth in SEQ ID NO:93, a CDR H2 including the sequence as set forth in SEQ ID NO:94, and a CDR H3 including the sequence as set forth in SEQ ID NO: 95; and wherein the light chain variable domain includes a CDR L1 including the sequence as set forth in SEQ ID NO:96, a CDR L2 including the sequence as set forth in SEQ ID NO:97 and a CDR L3 including the sequence as set forth in SEQ ID NO:98.

In embodiments, the anti-CD84 antibody is a chimeric antibody. In embodiments, the anti-CD84 antibody is a humanized antibody. In embodiments, the heavy chain variable domain includes a FR H1 as set forth in SEQ ID NO:99, a FR H2 as set forth in SEQ ID NO: 100, a FR H3 as set forth in SEQ ID NO: 101 and a FR H4 as set forth in SEQ ID NO:102. In embodiments, the light chain variable domain includes a FR L1 as set forth in SEQ ID NO:103, a FR L2 as set forth in SEQ ID NO: 104, a FR L3 as set forth in SEQ ID NO: 105 and a FR L4 as set forth in SEQ ID NO: 106. In embodiments, the heavy chain variable domain includes a FR H1 including the sequence as set forth in SEQ ID NO:99, a FR H2 including the sequence as set forth in SEQ ID NO: 100, a FR H3 including the sequence as set forth in SEQ ID NO: 101 and a FR H4 including the sequence as set forth in SEQ ID NO:102. In embodiments, the light chain variable domain includes a FR L1 including the sequence as set forth in SEQ ID NO: 103, a FR L2 including the sequence as set forth in SEQ ID NO:104, a FR L3 including the sequence as set forth in SEQ ID NO: 105 and a FR L4 including the sequence as set forth in SEQ ID NO:106.

In embodiments, the heavy chain variable domain includes the sequence of SEQ ID NO: 107. In embodiments, the heavy chain variable domain has the sequence of SEQ ID NO:107. In embodiments, the light chain variable domain includes the sequence of SEQ ID NO: 108. In embodiments, the light chain variable domain has the sequence of SEQ ID NO: 108. In embodiments, the anti-CD84 antibody includes a heavy chain including the sequence of SEQ ID NO: 109. In embodiments, the anti-CD84 antibody includes a heavy chain that has the sequence of SEQ ID NO:109. In embodiments, the anti-CD84 antibody includes a light chain including the sequence of SEQ ID NO:110. In embodiments, the anti-CD84 antibody includes a light chain that has the sequence of SEQ ID NO:110. In embodiments, the anti-CD84 antibody is referred to herein as clone 8A5.

In another aspect is provided an anti-CD84 antibody including a light chain variable domain and a heavy chain variable domain, wherein the heavy chain variable domain includes a CDR H1 as set forth in SEQ ID NO:1, a CDR H2 as set forth in SEQ ID NO:2, and a CDR H3 as set forth in SEQ ID NO:3; and wherein the light chain variable domain includes a CDR L1 as set forth in SEQ ID NO:4, a CDR L2 as set forth in SEQ ID NO:5 and a CDR L3 as set forth in SEQ ID NO: 6.

In another aspect is provided an anti-CD84 antibody including a light chain variable domain and a heavy chain variable domain, wherein the heavy chain variable domain includes a CDR H1 including the sequence as set forth in SEQ ID NO:1, a CDR H2 including the sequence as set forth in SEQ ID NO:2, and a CDR H3 including the sequence as set forth in SEQ ID NO:3; and wherein the light chain variable domain includes a CDR L1 including the sequence as set forth in SEQ ID NO:4, a CDR L2 including the sequence as set forth in SEQ ID NO:5 and a CDR L3 including the sequence as set forth in SEQ ID NO:6.

In embodiments, the anti-CD84 antibody is a chimeric antibody. In embodiments, the anti-CD84 antibody is a humanized antibody. In embodiments, the heavy chain variable domain includes a FR H1 as set forth in SEQ ID NO:7, a FR H2 as set forth in SEQ ID NO:8, a FR H3 as set forth in SEQ ID NO:9 and a FR H4 as set forth in SEQ ID NO: 10. In embodiments, the light chain variable domain includes a FR L1 as set forth in SEQ ID NO:11, a FR L2 as set forth in SEQ ID NO:12, a FR L3 as set forth in SEQ ID NO:13 and a FR L4 as set forth in SEQ ID NO:14. In embodiments, the heavy chain variable domain includes a FR H1 including the sequence as set forth in SEQ ID NO:7, a FR H2 including the sequence as set forth in SEQ ID NO:8, a FR H3 including the sequence as set forth in SEQ ID NO:9 and a FR H4 including the sequence as set forth in SEQ ID NO:10. In embodiments, the light chain variable domain includes a FR L1 including the sequence as set forth in SEQ ID NO: 11, a FR L2 including the sequence as set forth in SEQ ID NO:12, a FR L3 including the sequence as set forth in SEQ ID NO:13 and a FR L4 including the sequence as set forth in SEQ ID NO: 14.

In embodiments, the heavy chain variable domain includes the sequence of SEQ ID NO: 15. In embodiments, the heavy chain variable domain has the sequence of SEQ ID NO:15. In embodiments, the light chain variable domain includes the sequence of SEQ ID NO:16. In embodiments, the light chain variable domain has the sequence of SEQ ID NO: 16. In embodiments, the anti-CD84 antibody includes a heavy chain including the sequence of SEQ ID NO: 17. In embodiments, the anti-CD84 antibody includes a heavy chain that has the sequence of SEQ ID NO:17. In embodiments, the anti-CD84 antibody includes a light chain including the sequence of SEQ ID NO:18. In embodiments, the anti-CD84 antibody includes a light chain that has the sequence of SEQ ID NO:18. In embodiments, the anti-CD84 antibody is referred to herein as clone 4B11.

In embodiments, anti-CD84 antibody is a Fab′ fragment. In embodiments, the anti-CD84 antibody is an IgG. In embodiments, the anti-CD84 antibody is an IgG1, IgG2, IgG3, or IgG4. In embodiments, the anti-CD84 antibody is an IgG1. In embodiments, the anti-CD84 antibody is an IgG2. In embodiments, the anti-CD84 antibody is an IgG3. In embodiments, the anti-CD84 antibody is an IgG4. In embodiments, the light chain variable domain and the heavy chain variable domain form part of a scFv.

In embodiments, the anti-CD84 antibody is capable of binding a CD84 protein. In embodiments, the anti-CD84 antibody is bound to a CD84 protein. In embodiments, the CD84 protein is a human CD84 protein. In embodiments, the CD84 protein forms part of a cell. In embodiments, the CD84 protein is expressed on the surface of a cell. In embodiments, the cell is a cancer cell. In embodiments, the cancer cell is a leukemia cancer cell, a myeloid cancer cell, or a lymphoma cancer cell. In embodiments, the cancer cell is a leukemia cancer cell. In embodiments, the cancer cell is a myeloid cancer cell. In embodiments, the cancer cell is a lymphoma cancer cell.

In another aspect, there is provided an anti-CD84 antibody, wherein the anti-CD84 antibody binds the same epitope as an antibody including: a heavy chain variable domain including a CDR H1 as set forth in SEQ ID NO:19, a CDR H2 as set forth in SEQ ID NO:20, and a CDR H3 as set forth in SEQ ID NO:21, and a light chain variable domain including a CDR L1 as set forth in SEQ ID NO:22, a CDR L2 as set forth in SEQ ID NO:23 and a CDR L3 as set forth in SEQ ID NO:24.

In another aspect, there is provided an anti-CD84 antibody, wherein the anti-CD84 antibody binds the same epitope as an antibody including: a heavy chain variable domain including a CDR H1 as set forth in SEQ ID NO:39, a CDR H2 as set forth in SEQ ID NO:40, and a CDR H3 as set forth in SEQ ID NO:41, and a light chain variable domain including a CDR L1 as set forth in SEQ ID NO:42, a CDR L2 as set forth in SEQ ID NO:43 and a CDR L3 as set forth in SEQ ID NO:44.

In another aspect, there is provided an anti-CD84 antibody, wherein the anti-CD84 antibody binds the same epitope as an antibody including: a heavy chain variable domain including a CDR H1 as set forth in SEQ ID NO:57, a CDR H2 as set forth in SEQ ID NO:58, and a CDR H3 as set forth in SEQ ID NO:59, and a light chain variable domain including a CDR L1 as set forth in SEQ ID NO:60, a CDR L2 as set forth in SEQ ID NO:61 and a CDR L3 as set forth in SEQ ID NO:62.

In another aspect, there is provided an anti-CD84 antibody, wherein the anti-CD84 antibody binds the same epitope as an antibody including: a heavy chain variable domain including a CDR H1 as set forth in SEQ ID NO:75, a CDR H2 as set forth in SEQ ID NO:76, and a CDR H3 as set forth in SEQ ID NO:77, and a light chain variable domain including a CDR L1 as set forth in SEQ ID NO:78, a CDR L2 as set forth in SEQ ID NO:79 and a CDR L3 as set forth in SEQ ID NO:80.

In another aspect, there is provided an anti-CD84 antibody, wherein the anti-CD84 antibody binds the same epitope as an antibody including: a heavy chain variable domain including a CDR H1 as set forth in SEQ ID NO:93, a CDR H2 as set forth in SEQ ID NO:94, and a CDR H3 as set forth in SEQ ID NO:95, and a light chain variable domain including a CDR L1 as set forth in SEQ ID NO:96, a CDR L2 as set forth in SEQ ID NO:97 and a CDR L3 as set forth in SEQ ID NO:98.

In another aspect, there is provided an anti-CD84 antibody, wherein the anti-CD84 antibody binds the same epitope as an antibody including: a heavy chain variable domain including a CDR H1 as set forth in SEQ ID NO:1, a CDR H2 as set forth in SEQ ID NO:2, and a CDR H3 as set forth in SEQ ID NO:3, and a light chain variable domain including a CDR L1 as set forth in SEQ ID NO:4, a CDR L2 as set forth in SEQ ID NO:5 and a CDR L3 as set forth in SEQ ID NO:6.

In embodiments, the anti-CD84 antibody is attached to a therapeutic or a diagnostic moiety. In embodiments, the anti-CD84 antibody is attached to a therapeutic moiety. In embodiments, the anti-CD84 antibody is attached to a diagnostic moiety. In embodiments, the therapeutic moiety is an anti-cancer moiety.

As described above, a “light chain variable (VL) domain” as provided herein forms part of the variable region of the light chain of an antibody, an antibody variant or fragment thereof. Likewise, the “heavy chain variable (VH) domain” as provided herein forms part of the variable region of the heavy chain of an antibody, an antibody variant or fragment thereof. The light chain variable domain and the heavy chain variable domain together form the paratope, which binds an antigen (epitope). The paratope or antigen-binding site is formed at the N-terminus of an antibody, an antibody variant or fragment thereof. In embodiments, the light chain variable (VL) domain includes CDR L1, CDR L2, CDR L3 and FR L1, FR L2, FR L3 and FR L4 (framework regions) of an antibody light chain. In embodiments, the heavy chain variable (VH) domain includes CDR H1, CDR H2, CDR H3 and FR H1, FR H2, FR H3 and FR H4 (framework regions) of an antibody heavy chain. In embodiments, the light chain variable (VL) domain and a light chain constant (CL) domain form part of an antibody light chain. In embodiments, the heavy chain variable (VH) domain and a heavy chain constant (CH1) domain form part of an antibody heavy chain. In embodiments, the heavy chain variable (VH) domain and one or more heavy chain constant (CH1, CH2, or CH3) domains form part of an antibody heavy chain. Thus, in embodiments, the light chain variable (VL) domain forms part of an antibody. In embodiments, the heavy chain variable (VH) domain forms part of an antibody. In embodiments, the light chain variable (VL) domain forms part of a therapeutic antibody. In embodiments, the heavy chain variable (VH) domain forms part of a therapeutic antibody. In embodiments, the light chain variable (VL) domain forms part of a human antibody. In embodiments, the heavy chain variable (VH) domain forms part of a human antibody. In embodiments, the light chain variable (VL) domain forms part of a humanized antibody. In embodiments, the heavy chain variable (VH) domain forms part of a humanized antibody. In embodiments, the light chain variable (VL) domain forms part of a chimeric antibody. In embodiments, the heavy chain variable (VH) domain forms part of a chimeric antibody. In embodiments, the light chain variable (VL) domain forms part of an antibody fragment. In embodiments, the heavy chain variable (VH) domain forms part of an antibody fragment. In embodiments, the light chain variable (VL) domain forms part of an antibody variant. In embodiments, the heavy chain variable (VH) domain forms part of an antibody variant. In embodiments, the light chain variable (VL) domain forms part of a Fab. In embodiments, the heavy chain variable (VH) domain forms part of a Fab. In embodiments, the light chain variable (VL) domain forms part of a scFv. In embodiments, the heavy chain variable (VH) domain forms part of a scFv. In embodiments, the light chain variable (VL) domain forms part of a single domain antibody. In embodiments, the heavy chain variable (VH) domain forms part of a single domain antibody.

In one embodiment the heavy chain variable domain has the sequence of SEQ ID NO:15 and the light chain variable domain has the sequence of SEQ ID NO: 16. In one embodiment, the anti-CD84 antibody has a heavy chain of SEQ ID NO: 17 and a light chain of SEQ ID NO: 18. In one further embodiment, the antibody is 4B11.

In one embodiment the heavy chain variable domain has the sequence of SEQ ID NO:33 and the light chain variable domain has the sequence of SEQ ID NO:34. In one embodiment, the anti-CD84 antibody has a heavy chain of SEQ ID NO:35 and a light chain of SEQ ID NO:36. In one further embodiment, the antibody is 13D11.

In one embodiment, the anti-CD84 antibody has a heavy chain of SEQ ID NO:37 and a light chain of SEQ ID NO:38. In one further embodiment, the antibody is 13D11 Hu.

In one embodiment the heavy chain variable domain has the sequence of SEQ ID NO:53 and the light chain variable domain has the sequence of SEQ ID NO:54. In one embodiment, the anti-CD84 antibody has a heavy chain of SEQ ID NO:55 and a light chain of SEQ ID NO:56. In one further embodiment, the antibody is 2H7.

In one embodiment the heavy chain variable domain has the sequence of SEQ ID NO:71 and the light chain variable domain has the sequence of SEQ ID NO:72. In one embodiment, the anti-CD84 antibody has a heavy chain of SEQ ID NO:73 and a light chain of SEQ ID NO:74. In one further embodiment, the antibody is 5C6.

In one embodiment the heavy chain variable domain has the sequence of SEQ ID NO:89 and the light chain variable domain has the sequence of SEQ ID NO:90. In one embodiment, the anti-CD84 antibody has a heavy chain of SEQ ID NO:91 and a light chain of SEQ ID NO:92. In one further embodiment, the antibody is 7D2.

In one embodiment the heavy chain variable domain has the sequence of SEQ ID NO: 107 and the light chain variable domain has the sequence of SEQ ID NO: 108. In one embodiment, the anti-CD84 antibody has a heavy chain of SEQ ID NO: 109 and a light chain of SEQ ID NO:110. In one further embodiment, the antibody is 8A5.

### Cd84 Epitopes

The antibodies provided herein including embodiments thereof are capable of specifically binding to a CD84 epitope. In embodiments, the epitope is a human CD84 protein.

In embodiments, the epitope interacts with the heavy chain of the anti-CD84 antibody. In embodiments, the epitope includes a Val at a position corresponding to position 21 of SEQ ID NO: 111, an Asn at a position corresponding to position 22 of SEQ ID NO: 111, an Ile at a position corresponding to position 23 of SEQ ID NO: 111, a Gln at a position corresponding to position 24 of SEQ ID NO: 111, a Glu at a position corresponding to position 25 of SEQ ID NO: 111, a Pro at a position corresponding to position 26 of SEQ ID NO: 111, an Arg at a position corresponding to position 27 of SEQ ID NO: 111, a Gln at a position corresponding to position 28 of SEQ ID NO: 111, a Ser at a position corresponding to position 48 of SEQ ID NO: 111, a Glu at a position corresponding to position 49 of SEQ ID NO: 111, a Pro at a position corresponding to position 70 of SEQ ID NO: 111, a Tyr at a position corresponding to position 72 of SEQ ID NO: 111, an Ala at a position corresponding to position 95 of SEQ ID NO: 111, an Asp at a position corresponding to position 96 of SEQ ID NO: 111, a Pro at a position corresponding to position 97 of SEQ ID NO: 111, a Tyr at a position corresponding to position 98 of SEQ ID NO: 111, a Thr at a position corresponding to position 100 of SEQ ID NO: 111, or a Lys at a position corresponding to position 102 of SEQ ID NO: 111.

In embodiments, the epitope includes a Val at a position corresponding to position 21 of SEQ ID NO: 111. In embodiments, the epitope includes an Asn at a position corresponding to position 22 of SEQ ID NO: 111. In embodiments, the epitope includes an Ile at a position corresponding to position 23 of SEQ ID NO: 111. In embodiments, the epitope includes a Gln at a position corresponding to position 24 of SEQ ID NO: 111. In embodiments, the epitope includes a Glu at a position corresponding to position 25 of SEQ ID NO: 111. In embodiments, the epitope includes a Pro at a position corresponding to position 26 of SEQ ID NO: 111. In embodiments, the epitope includes an Arg at a position corresponding to position 27 of SEQ ID NO: 111. In embodiments, the epitope includes a Gln at a position corresponding to position 28 of SEQ ID NO: 111. In embodiments, the epitope includes a Ser at a position corresponding to position 48 of SEQ ID NO: 111. In embodiments, the epitope includes a Glu at a position corresponding to position 49 of SEQ ID NO: 111. In embodiments, the epitope includes a Pro at a position corresponding to position 70 of SEQ ID NO: 111. In embodiments, the epitope includes a Tyr at a position corresponding to position 72 of SEQ ID NO: 111. In embodiments, the epitope includes an Ala at a position corresponding to position 95 of SEQ ID NO: 111. In embodiments, the epitope includes an Asp at a position corresponding to position 96 of SEQ ID NO: 111. In embodiments, the epitope includes a Pro at a position corresponding to position 97 of SEQ ID NO: 111. In embodiments, the epitope includes a Tyr at a position corresponding to position 98 of SEQ ID NO: 111. In embodiments, the epitope includes a Thr at a position corresponding to position 100 of SEQ ID NO: 111. In embodiments, the epitope includes or a Lys at a position corresponding to position 102 of SEQ ID NO: 111.

In embodiments, the epitope includes a Val at a position corresponding to position 21 of SEQ ID NO: 111, an Asn at a position corresponding to position 22 of SEQ ID NO: 111, an Ile at a position corresponding to position 23 of SEQ ID NO: 111, a Gln at a position corresponding to position 24 of SEQ ID NO: 111, a Glu at a position corresponding to position 25 of SEQ ID NO: 111, a Pro at a position corresponding to position 26 of SEQ ID NO: 111, an Arg at a position corresponding to position 27 of SEQ ID NO: 111, a Gln at a position corresponding to position 28 of SEQ ID NO: 111, a Ser at a position corresponding to position 48 of SEQ ID NO: 111, a Glu at a position corresponding to position 49 of SEQ ID NO: 111, a Pro at a position corresponding to position 70 of SEQ ID NO: 111, a Tyr at a position corresponding to position 72 of SEQ ID NO: 111, an Ala at a position corresponding to position 95 of SEQ ID NO: 111, an Asp at a position corresponding to position 96 of SEQ ID NO: 111, a Pro at a position corresponding to position 97 of SEQ ID NO: 111, a Tyr at a position corresponding to position 98 of SEQ ID NO: 111, a Thr at a position corresponding to position 100 of SEQ ID NO: 111, and a Lys at a position corresponding to position 102 of SEQ ID NO: 111.

In one embodiment, the heavy chain of the anti-CD84 antibody binds the epitope with a Thr at a position corresponding to position 30 of SEQ ID NO:109, a Thr at a position corresponding to position 31 of SEQ ID NO: 109, a Tyr at a position corresponding to position 32 of SEQ ID NO: 109, a Ser at a position corresponding to position 33 of SEQ ID NO:109, a His at a position corresponding to position 35 of SEQ ID NO: 109, a Trp at a position corresponding to position 47 of SEQ ID NO: 109, a Trp at a position corresponding to position 50 of SEQ ID NO: 109, an Asp at a position corresponding to position 52 of SEQ ID NO:109, a Thr at a position corresponding to position 53 of SEQ ID NO:109, an Ala at a position corresponding to position 54 of SEQ ID NO:109, a Thr at a position corresponding to position 55 of SEQ ID NO:109, a Glu at a position corresponding to position 57 of SEQ ID NO:109, a Pro at a position corresponding to position 58 of SEQ ID NO:109, a Thr at a position corresponding to position 59 of SEQ ID NO: 109, a Tyr at a position corresponding to position 60 of SEQ ID NO:109, an Ala at a position corresponding to position 61 of SEQ ID NO: 109, a Lys at a position corresponding to position 65 of SEQ ID NO: 109, a Ser at a position corresponding to position 99 of SEQ ID NO:109, a Tyr at a position corresponding to position 101 of SEQ ID NO:109, a Trp at a position corresponding to position 102 of SEQ ID NO: 109, a Tyr at a position corresponding to position 103 of SEQ ID NO: 109, or a Phe at a position corresponding to position 104 of SEQ ID NO:109.

In one further embodiment, the heavy chain of the anti-CD84 antibody binds the epitope with a Thr at a position corresponding to position 30 of SEQ ID NO: 109, a Thr at a position corresponding to position 31 of SEQ ID NO: 109, a Tyr at a position corresponding to position 32 of SEQ ID NO:109, a Ser at a position corresponding to position 33 of SEQ ID NO:109, a His at a position corresponding to position 35 of SEQ ID NO: 109, a Trp at a position corresponding to position 47 of SEQ ID NO:109, a Trp at a position corresponding to position 50 of SEQ ID NO: 109, an Asp at a position corresponding to position 52 of SEQ ID NO:109, a Thr at a position corresponding to position 53 of SEQ ID NO: 109, an Ala at a position corresponding to position 54 of SEQ ID NO: 109, a Thr at a position corresponding to position 55 of SEQ ID NO:109, a Glu at a position corresponding to position 57 of SEQ ID NO:109, a Pro at a position corresponding to position 58 of SEQ ID NO:109, a Thr at a position corresponding to position 59 of SEQ ID NO: 109, a Tyr at a position corresponding to position 60 of SEQ ID NO:109, an Ala at a position corresponding to position 61 of SEQ ID NO:109, a Lys at a position corresponding to position 65 of SEQ ID NO:109, a Ser at a position corresponding to position 99 of SEQ ID NO:109, a Tyr at a position corresponding to position 101 of SEQ ID NO:109, a Trp at a position corresponding to position 102 of SEQ ID NO: 109, a Tyr at a position corresponding to position 103 of SEQ ID NO: 109, and a Phe at a position corresponding to position 104 of SEQ ID NO:109.

In one embodiment, the heavy chain of the anti-CD84 antibody binds a Gln at a position corresponding to position 24 of SEQ ID NO:111 with a Ser at a position corresponding to position 99 of SEQ ID NO:109. In one embodiment, the heavy chain of the anti-CD84 antibody binds a Gln at a position corresponding to position 24 of SEQ ID NO:111 with a Thr at a position corresponding to position 53 of SEQ ID NO: 109. In one embodiment, the heavy chain of the anti-CD84 antibody binds a Glu at a position corresponding to position 25 of SEQ ID NO:111 with a Ser at a position corresponding to position 33 of SEQ ID NO:109. In one embodiment, the heavy chain of the anti-CD84 antibody binds a Gln at a position corresponding to position 24 of SEQ ID NO: 111 with a Thr at a position corresponding to position 30 of SEQ ID NO:109.

In embodiments, the epitope interacts with the light chain of the anti-CD84 antibody. In embodiments, the epitope includes a Glu at a position corresponding to position 25 of SEQ ID NO: 111, an Arg at a position corresponding to position 27 of SEQ ID NO: 111, a Gln at a position corresponding to position 28 of SEQ ID NO: 111, a Val at a position corresponding to position 29 of SEQ ID NO: 111, a Lys at a position corresponding to position 30 of SEQ ID NO: 111, a Pro at a position corresponding to position 45 of SEQ ID NO: 111, a Gly at a position corresponding to position 46 of SEQ ID NO: 111, a Ser at a position corresponding to position 48 of SEQ ID NO: 111, a Glu at a position corresponding to position 49 of SEQ ID NO: 111, a Gln at a position corresponding to position 94 of SEQ ID NO: 111, or an Ala at a position corresponding to position 95 of SEQ ID NO: 111.

In embodiments, the epitope includes a Glu at a position corresponding to position 25 of SEQ ID NO: 111. In embodiments, the epitope includes an Arg at a position corresponding to position 27 of SEQ ID NO: 111. In embodiments, the epitope includes a Gln at a position corresponding to position 28 of SEQ ID NO: 111. In embodiments, the epitope includes a Val at a position corresponding to position 29 of SEQ ID NO: 111. In embodiments, the epitope includes a Lys at a position corresponding to position 30 of SEQ ID NO: 111. In embodiments, the epitope includes a Pro at a position corresponding to position 45 of SEQ ID NO: 111. In embodiments, the epitope includes a Gly at a position corresponding to position 46 of SEQ ID NO: 111. In embodiments, the epitope includes a Ser at a position corresponding to position 48 of SEQ ID NO: 111. In embodiments, the epitope includes a Glu at a position corresponding to position 49 of SEQ ID NO: 111. In embodiments, the epitope includes a Gln at a position corresponding to position 94 of SEQ ID NO: 111. In embodiments, the epitope includes an Ala at a position corresponding to position 95 of SEQ ID NO: 111.

In embodiments, the epitope includes a Glu at a position corresponding to position 25 of SEQ ID NO: 111, an Arg at a position corresponding to position 27 of SEQ ID NO: 111, a Gln at a position corresponding to position 28 of SEQ ID NO: 111, a Val at a position corresponding to position 29 of SEQ ID NO: 111, a Lys at a position corresponding to position 30 of SEQ ID NO: 111, a Pro at a position corresponding to position 45 of SEQ ID NO: 111, a Gly at a position corresponding to position 46 of SEQ ID NO: 111, a Ser at a position corresponding to position 48 of SEQ ID NO: 111, a Glu at a position corresponding to position 49 of SEQ ID NO: 111, a Gln at a position corresponding to position 94 of SEQ ID NO: 111, and an Ala at a position corresponding to position 95 of SEQ ID NO: 111.

In one embodiment, the light chain of the anti-CD84 antibody binds the epitope with a His at a position corresponding to position 31 of SEQ ID NO:110, an Ile at a position corresponding to position 32 of SEQ ID NO:110, an Asn at a position corresponding to position 33 of SEQ ID NO:110, a Gly at a position corresponding to position 34 of SEQ ID NO:110, an Asn at a position corresponding to position 35 of SEQ ID NO:110, a Tyr at a position corresponding to position 37 of SEQ ID NO:110, a Glu at a position corresponding to position 39 of SEQ ID NO: 110, a Lys at a position corresponding to position 55 of SEQ ID NO:110, a Phe at a position corresponding to position 94 of SEQ ID NO:110, a Gly at a position corresponding to position 96 of SEQ ID NO: 110, a Ser at a position corresponding to position 97 of SEQ ID NO: 110, a His at a position corresponding to position 98 of SEQ ID NO:110, a Val at a position corresponding to position 99 of SEQ ID NO: 110, or a Trp at a position corresponding to position 101 of SEQ ID NO:110.

In one embodiment, the light chain of the anti-CD84 antibody binds the epitope with a His at a position corresponding to position 31 of SEQ ID NO: 110, an Ile at a position corresponding to position 32 of SEQ ID NO:110, an Asn at a position corresponding to position 33 of SEQ ID NO:110, a Gly at a position corresponding to position 34 of SEQ ID NO:110, an Asn at a position corresponding to position 35 of SEQ ID NO:110, a Tyr at a position corresponding to position 37 of SEQ ID NO: 110, a Glu at a position corresponding to position 39 of SEQ ID NO: 110, a Lys at a position corresponding to position 55 of SEQ ID NO:110, a Phe at a position corresponding to position 94 of SEQ ID NO:110, a Gly at a position corresponding to position 96 of SEQ ID NO:110, a Ser at a position corresponding to position 97 of SEQ ID NO: 110, a His at a position corresponding to position 98 of SEQ ID NO:110, a Val at a position corresponding to position 99 of SEQ ID NO:110, and a Trp at a position corresponding to position 101 of SEQ ID NO:110.

In one embodiment, the light chain of the anti-CD84 antibody binds a Ser at a position corresponding to position 48 of SEQ ID NO:111 with an Asn at a position corresponding to position 33 of SEQ ID NO:110. In one embodiment, the light chain of the anti-CD84 antibody binds a Glu at a position corresponding to position 49 of SEQ ID NO:111 with an Lys at a position corresponding to position 55 of SEQ ID NO:110. In one embodiment, the light chain of the anti-CD84 antibody binds an Arg at a position corresponding to position 27 of SEQ ID NO:111 with a Gly at a position corresponding to position 96 of SEQ ID NO:110. In one embodiment, the light chain of the anti-CD84 antibody binds a Ser at a position corresponding to position 48 of SEQ ID NO: 111 with an Asn at a position corresponding to position 35 of SEQ ID NO:110. In one embodiment, the light chain of the anti-CD84 antibody binds a Glu at a position corresponding to position 49 of SEQ ID NO:111 with a Lys at a position corresponding to position 55 of SEQ ID NO: 110. In one embodiment, the light chain of the anti-CD84 antibody binds an Arg at a position corresponding to position 27 of SEQ ID NO: 111 with a Glu at a position corresponding to position 39 of SEQ ID NO:110.

### Nucleic Acid Compositions

The compositions provided herein include nucleic acid molecules encoding the anti-CD84 antibodies or portions thereof as provided herein including embodiments thereof. The antibodies encoded by the isolated nucleic acid as provided herein are described in detail throughout this application (including the description above and in the examples section). Thus, in an aspect is provided isolated nucleic acid encoding an anti-CD84 antibody as provided herein including embodiments thereof.

### Cell Compositions

In another aspect, there is provided a cell including an anti-CD84 antibody as provided herein including embodiments thereof, or a nucleic acid as provided here in including embodiments thereof.

### Pharmaceutical Compositions

The compositions provided herein include pharmaceutical compositions including the anti-CD84 antibodies or portions thereof as provided herein including embodiments thereof. Thus, in an aspect is provided a pharmaceutical composition including a therapeutically effective amount of an antibody as provided herein including embodiments thereof and a pharmaceutically acceptable excipient.

### Methods of Producing an Antibody

In an aspect, there is provided a method of forming an antibody capable of binding to CD84, the method including immunizing a mammal with a peptide including the sequence of SEQ ID NO: 111.

### Methods of Treatment

The compositions (e.g., the anti-CD84 antibodies) provided herein, including embodiments thereof, are contemplated as providing effective treatments for diseases such as cancer (e.g., acute myeloid leukemia). Thus, in an aspect is provided a method of treating cancer in a subject in need thereof, said method includes administering to a subject a therapeutically effective amount of an anti-CD84 antibody as provided herein including embodiments thereof or a pharmaceutical composition as provided herein including embodiments thereof, thereby treating cancer in said subject. In embodiments, the cancer is lymphoma, myeloma, and/or leukemia. In embodiments, the cancer is lymphoma, myeloma, and leukemia. In embodiments, the cancer is lymphoma. In embodiments, the cancer is myeloma. In embodiments, the cancer is leukemia. In embodiments, the cancer is acute myeloid leukemia (AML).

In embodiments, the anti-CD84 antibody is administered at an amount that is less than the amount of an anti-cancer antibody normally administered to treat a cancer. In embodiments, the amount is 2, 5, 10, 100, 200, 500, 1000, or 10000 times less than the amount of an anti-cancer antibody. In embodiments, the amount is 2 times less than the amount of an anti-cancer antibody. In embodiments, the amount is 5 times less than the amount of an anti-cancer antibody. In embodiments, the amount is 10 times less than the amount of an anti-cancer antibody. In embodiments, the amount is 100 times less than the amount of an anti-cancer antibody. In embodiments, the amount is 200 times less than the amount of an anti-cancer antibody. In embodiments, the amount is 500 times less than the amount of an anti-cancer antibody. In embodiments, the amount is 1000 times less than the amount of an anti-cancer antibody. In embodiments, the amount is 10000 times less than the amount of an anti-cancer antibody.

In embodiments, the anti-CD84 antibody is administered at an amount from about 0.5 mg/kg to about 20 mg/kg. In embodiments, the anti-CD84 antibody is administered at an amount from about 1 mg/kg to about 20 mg/kg. In embodiments, the anti-CD84 antibody is administered at an amount from about 1.5 mg/kg to about 20 mg/kg. In embodiments, the anti-CD84 antibody is administered at an amount from about 2 mg/kg to about 20 mg/kg. In embodiments, the anti-CD84 antibody is administered at an amount from about 2.5 mg/kg to about 20 mg/kg. In embodiments, the anti-CD84 antibody is administered at an amount from about 3 mg/kg to about 20 mg/kg. In embodiments, the anti-CD84 antibody is administered at an amount from about 3.5 mg/kg to about 20 mg/kg. In embodiments, the anti-CD84 antibody is administered at an amount from about 4 mg/kg to about 20 mg/kg. In embodiments, the anti-CD84 antibody is administered at an amount from about 4.5 mg/kg to about 20 mg/kg. In embodiments, the anti-CD84 antibody is administered at an amount from about 5 mg/kg to about 20 mg/kg. In embodiments, the anti-CD84 antibody is administered at an amount from about 5.5 mg/kg to about 20 mg/kg. In embodiments, the anti-CD84 antibody is administered at an amount from about 6 mg/kg to about 20 mg/kg. In embodiments, the anti-CD84 antibody is administered at an amount from about 6.5 mg/kg to about 20 mg/kg. In embodiments, the anti-CD84 antibody is administered at an amount from about 7 mg/kg to about 20 mg/kg. In embodiments, the anti-CD84 antibody is administered at an amount from about 7.5 mg/kg to about 20 mg/kg. In embodiments, the anti-CD84 antibody is administered at an amount from about 8 mg/kg to about 20 mg/kg. In embodiments, the anti-CD84 antibody is administered at an amount from about 8.5 mg/kg to about 20 mg/kg. In embodiments, the anti-CD84 antibody is administered at an amount from about 9 mg/kg to about 20 mg/kg. In embodiments, the anti-CD84 antibody is administered at an amount from about 9.5 mg/kg to about 20 mg/kg. In embodiments, the anti-CD84 antibody is administered at an amount from about 10 mg/kg to about 20 mg/kg.

In embodiments, the anti-CD84 antibody is administered at an amount from about 10.5 mg/kg to about 20 mg/kg. In embodiments, the anti-CD84 antibody is administered at an amount from about 11 mg/kg to about 20 mg/kg. In embodiments, the anti-CD84 antibody is administered at an amount from about 11.5 mg/kg to about 20 mg/kg. In embodiments, the anti-CD84 antibody is administered at an amount from about 12 mg/kg to about 20 mg/kg. In embodiments, the anti-CD84 antibody is administered at an amount from about 12.5 mg/kg to about 20 mg/kg. In embodiments, the anti-CD84 antibody is administered at an amount from about 13 mg/kg to about 20 mg/kg. In embodiments, the anti-CD84 antibody is administered at an amount from about 13.5 mg/kg to about 20 mg/kg. In embodiments, the anti-CD84 antibody is administered at an amount from about 14 mg/kg to about 20 mg/kg. In embodiments, the anti-CD84 antibody is administered at an amount from about 14.5 mg/kg to about 20 mg/kg. In embodiments, the anti-CD84 antibody is administered at an amount from about 15 mg/kg to about 20 mg/kg. In embodiments, the anti-CD84 antibody is administered at an amount from about 15.5 mg/kg to about 20 mg/kg. In embodiments, the anti-CD84 antibody is administered at an amount from about 6 mg/kg to about 20 mg/kg. In embodiments, the anti-CD84 antibody is administered at an amount from about 16.5 mg/kg to about 20 mg/kg. In embodiments, the anti-CD84 antibody is administered at an amount from about 17 mg/kg to about 20 mg/kg. In embodiments, the anti-CD84 antibody is administered at an amount from about 17.5 mg/kg to about 20 mg/kg. In embodiments, the anti-CD84 antibody is administered at an amount from about 18 mg/kg to about 20 mg/kg. In embodiments, the anti-CD84 antibody is administered at an amount from about 18.5 mg/kg to about 20 mg/kg. In embodiments, the anti-CD84 antibody is administered at an amount from about 19 mg/kg to about 20 mg/kg. In embodiments, the anti-CD84 antibody is administered at an amount from about 19.5 mg/kg to about 20 mg/kg.

In embodiments, the anti-CD84 antibody is administered at an amount of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 mg/kg. In embodiments, the anti-CD84 antibody is administered at an amount of about 0.5 mg/kg. In embodiments, the anti-CD84 antibody is administered at an amount of about 1 mg/kg. In embodiments, the anti-CD84 antibody is administered at an amount of about 1.5 mg/kg. In embodiments, the anti-CD84 antibody is administered at an amount of about 2 mg/kg. In embodiments, the anti-CD84 antibody is administered at an amount of about 2.5 mg/kg. In embodiments, the anti-CD84 antibody is administered at an amount of about 3 mg/kg. In embodiments, the anti-CD84 antibody is administered at an amount of about 3.5 mg/kg. In embodiments, the anti-CD84 antibody is administered at an amount of about 4 mg/kg. In embodiments, the anti-CD84 antibody is administered at an amount of about 4.5 mg/kg. In embodiments, the anti-CD84 antibody is administered at an amount of about 5 mg/kg. In embodiments, the anti-CD84 antibody is administered at an amount of about 5.5 mg/kg. In embodiments, the anti-CD84 antibody is administered at an amount of about 6 mg/kg. In embodiments, the anti-CD84 antibody is administered at an amount of about 6.5 mg/kg. In embodiments, the anti-CD84 antibody is administered at an amount of about 7 mg/kg. In embodiments, the anti-CD84 antibody is administered at an amount of about 7.5 mg/kg. In embodiments, the anti-CD84 antibody is administered at an amount of about 8 mg/kg. In embodiments, the anti-CD84 antibody is administered at an amount of about 8.5 mg/kg. In embodiments, the anti-CD84 antibody is administered at an amount of about 9 mg/kg. In embodiments, the anti-CD84 antibody is administered at an amount of about 9.5 mg/kg. In embodiments, the anti-CD84 antibody is administered at an amount of about 10 mg/kg. In embodiments, the anti-CD84 antibody is administered at an amount of about 10.5 mg/kg. In embodiments, the anti-CD84 antibody is administered at an amount of about 11 mg/kg. In embodiments, the anti-CD84 antibody is administered at an amount of about 11.5 mg/kg. In embodiments, the anti-CD84 antibody is administered at an amount of about 12 mg/kg. In embodiments, the anti-CD84 antibody is administered at an amount of about 12.5 mg/kg. In embodiments, the anti-CD84 antibody is administered at an amount of about 13 mg/kg. In embodiments, the anti-CD84 antibody is administered at an amount of about 13.5 mg/kg. In embodiments, the anti-CD84 antibody is administered at an amount of about 14 mg/kg. In embodiments, the anti-CD84 antibody is administered at an amount of about 14.5 mg/kg. In embodiments, the anti-CD84 antibody is administered at an amount of about 15 mg/kg. In embodiments, the anti-CD84 antibody is administered at an amount of about 15.5 mg/kg. In embodiments, the anti-CD84 antibody is administered at an amount of about 16 mg/kg. In embodiments, the anti-CD84 antibody is administered at an amount of about 16.5 mg/kg. In embodiments, the anti-CD84 antibody is administered at an amount of about 17 mg/kg. In embodiments, the anti-CD84 antibody is administered at an amount of about 17.5 mg/kg. In embodiments, the anti-CD84 antibody is administered at an amount of about 18 mg/kg. In embodiments, the anti-CD84 antibody is administered at an amount of about 18.5 mg/kg. In embodiments, the anti-CD84 antibody is administered at an amount of about 19 mg/kg. In embodiments, the anti-CD84 antibody is administered at an amount of about 19.5 mg/kg. In embodiments, the anti-CD84 antibody is administered at an amount of about 20 mg/kg.

In embodiments, the anti-CD84 antibody is administered at an amount from 0.5 mg/kg to 20 mg/kg. In embodiments, the anti-CD84 antibody is administered at an amount from 1 mg/kg to 20 mg/kg. In embodiments, the anti-CD84 antibody is administered at an amount from 1.5 mg/kg to 20 mg/kg. In embodiments, the anti-CD84 antibody is administered at an amount from 2 mg/kg to 20 mg/kg. In embodiments, the anti-CD84 antibody is administered at an amount from 2.5 mg/kg to 20 mg/kg. In embodiments, the anti-CD84 antibody is administered at an amount from 3 mg/kg to 20 mg/kg. In embodiments, the anti-CD84 antibody is administered at an amount from 3.5 mg/kg to 20 mg/kg. In embodiments, the anti-CD84 antibody is administered at an amount from 4 mg/kg to 20 mg/kg. In embodiments, the anti-CD84 antibody is administered at an amount from 4.5 mg/kg to 20 mg/kg. In embodiments, the anti-CD84 antibody is administered at an amount from 5 mg/kg to 20 mg/kg. In embodiments, the anti-CD84 antibody is administered at an amount from 5.5 mg/kg to 20 mg/kg. In embodiments, the anti-CD84 antibody is administered at an amount from 6 mg/kg to 20 mg/kg. In embodiments, the anti-CD84 antibody is administered at an amount from 6.5 mg/kg to 20 mg/kg. In embodiments, the anti-CD84 antibody is administered at an amount from 7 mg/kg to 20 mg/kg. In embodiments, the anti-CD84 antibody is administered at an amount from 7.5 mg/kg to 20 mg/kg. In embodiments, the anti-CD84 antibody is administered at an amount from 8 mg/kg to 20 mg/kg. In embodiments, the anti-CD84 antibody is administered at an amount from 8.5 mg/kg to 20 mg/kg. In embodiments, the anti-CD84 antibody is administered at an amount from 9 mg/kg to 20 mg/kg. In embodiments, the anti-CD84 antibody is administered at an amount from 9.5 mg/kg to 20 mg/kg. In embodiments, the anti-CD84 antibody is administered at an amount from 10 mg/kg to 20 mg/kg.

In embodiments, the anti-CD84 antibody is administered at an amount from 10.5 mg/kg to 20 mg/kg. In embodiments, the anti-CD84 antibody is administered at an amount from 11 mg/kg to 20 mg/kg. In embodiments, the anti-CD84 antibody is administered at an amount from 11.5 mg/kg to 20 mg/kg. In embodiments, the anti-CD84 antibody is administered at an amount from 12 mg/kg to 20 mg/kg. In embodiments, the anti-CD84 antibody is administered at an amount from 12.5 mg/kg to 20 mg/kg. In embodiments, the anti-CD84 antibody is administered at an amount from 13 mg/kg to 20 mg/kg. In embodiments, the anti-CD84 antibody is administered at an amount from 13.5 mg/kg to 20 mg/kg. In embodiments, the anti-CD84 antibody is administered at an amount from 14 mg/kg to 20 mg/kg. In embodiments, the anti-CD84 antibody is administered at an amount from 14.5 mg/kg to 20 mg/kg. In embodiments, the anti-CD84 antibody is administered at an amount from 15 mg/kg to 20 mg/kg. In embodiments, the anti-CD84 antibody is administered at an amount from 15.5 mg/kg to 20 mg/kg. In embodiments, the anti-CD84 antibody is administered at an amount from 6 mg/kg to 20 mg/kg. In embodiments, the anti-CD84 antibody is administered at an amount from 16.5 mg/kg to 20 mg/kg. In embodiments, the anti-CD84 antibody is administered at an amount from 17 mg/kg to 20 mg/kg. In embodiments, the anti-CD84 antibody is administered at an amount from 17.5 mg/kg to 20 mg/kg. In embodiments, the anti-CD84 antibody is administered at an amount from 18 mg/kg to 20 mg/kg. In embodiments, the anti-CD84 antibody is administered at an amount from 18.5 mg/kg to 20 mg/kg. In embodiments, the anti-CD84 antibody is administered at an amount from 19 mg/kg to 20 mg/kg. In embodiments, the anti-CD84 antibody is administered at an amount from 19.5 mg/kg to 20 mg/kg.

In embodiments, the anti-CD84 antibody is administered at an amount of 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 mg/kg. In embodiments, the anti-CD84 antibody is administered at an amount of about 0.5 mg/kg. In embodiments, the anti-CD84 antibody is administered at an amount of about 1 mg/kg. In embodiments, the anti-CD84 antibody is administered at an amount of about 1.5 mg/kg. In embodiments, the anti-CD84 antibody is administered at an amount of about 2 mg/kg. In embodiments, the anti-CD84 antibody is administered at an amount of about 2.5 mg/kg. In embodiments, the anti-CD84 antibody is administered at an amount of about 3 mg/kg. In embodiments, the anti-CD84 antibody is administered at an amount of about 3.5 mg/kg. In embodiments, the anti-CD84 antibody is administered at an amount of about 4 mg/kg. In embodiments, the anti-CD84 antibody is administered at an amount of about 4.5 mg/kg. In embodiments, the anti-CD84 antibody is administered at an amount of about 5 mg/kg. In embodiments, the anti-CD84 antibody is administered at an amount of about 5.5 mg/kg. In embodiments, the anti-CD84 antibody is administered at an amount of about 6 mg/kg. In embodiments, the anti-CD84 antibody is administered at an amount of about 6.5 mg/kg. In embodiments, the anti-CD84 antibody is administered at an amount of about 7 mg/kg. In embodiments, the anti-CD84 antibody is administered at an amount of about 7.5 mg/kg. In embodiments, the anti-CD84 antibody is administered at an amount of about 8 mg/kg. In embodiments, the anti-CD84 antibody is administered at an amount of about 8.5 mg/kg. In embodiments, the anti-CD84 antibody is administered at an amount of about 9 mg/kg. In embodiments, the anti-CD84 antibody is administered at an amount of about 9.5 mg/kg. In embodiments, the anti-CD84 antibody is administered at an amount of about 10 mg/kg. In embodiments, the anti-CD84 antibody is administered at an amount of about 10.5 mg/kg. In embodiments, the anti-CD84 antibody is administered at an amount of about 11 mg/kg. In embodiments, the anti-CD84 antibody is administered at an amount of about 11.5 mg/kg. In embodiments, the anti-CD84 antibody is administered at an amount of about 12 mg/kg. In embodiments, the anti-CD84 antibody is administered at an amount of about 12.5 mg/kg. In embodiments, the anti-CD84 antibody is administered at an amount of about 13 mg/kg. In embodiments, the anti-CD84 antibody is administered at an amount of about 13.5 mg/kg. In embodiments, the anti-CD84 antibody is administered at an amount of about 14 mg/kg. In embodiments, the anti-CD84 antibody is administered at an amount of about 14.5 mg/kg. In embodiments, the anti-CD84 antibody is administered at an amount of about 15 mg/kg. In embodiments, the anti-CD84 antibody is administered at an amount of about 15.5 mg/kg. In embodiments, the anti-CD84 antibody is administered at an amount of about 16 mg/kg. In embodiments, the anti-CD84 antibody is administered at an amount of about 16.5 mg/kg. In embodiments, the anti-CD84 antibody is administered at an amount of about 17 mg/kg. In embodiments, the anti-CD84 antibody is administered at an amount of about 17.5 mg/kg. In embodiments, the anti-CD84 antibody is administered at an amount of about 18 mg/kg. In embodiments, the anti-CD84 antibody is administered at an amount of about 18.5 mg/kg. In embodiments, the anti-CD84 antibody is administered at an amount of about 19 mg/kg. In embodiments, the anti-CD84 antibody is administered at an amount of about 19.5 mg/kg. In embodiments, the anti-CD84 antibody is administered at an amount of about 20 mg/kg. The term “mg/kg” as provided herein refers to the mg of anti-CD84 antibody or salt thereof per kg body weight.

In embodiments, the anti-CD84 antibody is administered at an amount from about 0.5 mg/kg to about 19.5 mg/kg. In embodiments, the anti-CD84 antibody is administered at an amount from about 0.5 mg/kg to about 19 mg/kg. In embodiments, the anti-CD84 antibody is administered at an amount from about 0.5 mg/kg to about 18.5 mg/kg. In embodiments, the anti-CD84 antibody is administered at an amount from about 0.5 mg/kg to about 18 mg/kg. In embodiments, the anti-CD84 antibody is administered at an amount from about 0.5 mg/kg to about 17.5 mg/kg. In embodiments, the anti-CD84 antibody is administered at an amount from about 0.5 mg/kg to about 17 mg/kg. In embodiments, the anti-CD84 antibody is administered at an amount from about 0.5 mg/kg to about 16.5 mg/kg. In embodiments, the anti-CD84 antibody is administered at an amount from about 0.5 mg/kg to about 16 mg/kg. In embodiments, the anti-CD84 antibody is administered at an amount from about 0.5 mg/kg to about 15.5 mg/kg. In embodiments, the anti-CD84 antibody is administered at an amount from about 0.5 mg/kg to about 15 mg/kg. In embodiments, the anti-CD84 antibody is administered at an amount from about 0.5 mg/kg to about 14.5 mg/kg. In embodiments, the anti-CD84 antibody is administered at an amount from about 0.5 mg/kg to about 14 mg/kg. In embodiments, the anti-CD84 antibody is administered at an amount from about 0.5 mg/kg to about 13.5 mg/kg. In embodiments, the anti-CD84 antibody is administered at an amount from about 0.5 mg/kg to about 13 mg/kg. In embodiments, the anti-CD84 antibody is administered at an amount from about 0.5 mg/kg to about 12.5 mg/kg. In embodiments, the anti-CD84 antibody is administered at an amount from about 0.5 mg/kg to about 12 mg/kg. In embodiments, the anti-CD84 antibody is administered at an amount from about 0.5 mg/kg to about 11.5 mg/kg. In embodiments, the anti-CD84 antibody is administered at an amount from about 0.5 mg/kg to about 11 mg/kg. In embodiments, the anti-CD84 antibody is administered at an amount from about 0.5 mg/kg to about 10.5 mg/kg. In embodiments, the anti-CD84 antibody is administered at an amount from about 0.5 mg/kg to about 10 mg/kg.

In embodiments, the anti-CD84 antibody is administered at an amount from about 0.5 mg/kg to about 9.5 mg/kg. In embodiments, the anti-CD84 antibody is administered at an amount from about 0.5 mg/kg to about 9 mg/kg. In embodiments, the anti-CD84 antibody is administered at an amount from about 0.5 mg/kg to about 8.5 mg/kg. In embodiments, the anti-CD84 antibody is administered at an amount from about 0.5 mg/kg to about 8 mg/kg. In embodiments, the anti-CD84 antibody is administered at an amount from about 0.5 mg/kg to about 7.5 mg/kg. In embodiments, the anti-CD84 antibody is administered at an amount from about 0.5 mg/kg to about 7 mg/kg. In embodiments, the anti-CD84 antibody is administered at an amount from about 0.5 mg/kg to about 6.5 mg/kg. In embodiments, the anti-CD84 antibody is administered at an amount from about 0.5 mg/kg to about 6 mg/kg. In embodiments, the anti-CD84 antibody is administered at an amount from about 0.5 mg/kg to about 5.5 mg/kg. In embodiments, the anti-CD84 antibody is administered at an amount from about 0.5 mg/kg to about 5 mg/kg. In embodiments, the anti-CD84 antibody is administered at an amount from about 0.5 mg/kg to about 4.5 mg/kg. In embodiments, the anti-CD84 antibody is administered at an amount from about 0.5 mg/kg to about 4 mg/kg. In embodiments, the anti-CD84 antibody is administered at an amount from about 0.5 mg/kg to about 3.5 mg/kg. In embodiments, the anti-CD84 antibody is administered at an amount from about 0.5 mg/kg to about 3 mg/kg. In embodiments, the anti-CD84 antibody is administered at an amount from about 0.5 mg/kg to about 2.5 mg/kg. In embodiments, the anti-CD84 antibody is administered at an amount from about 0.5 mg/kg to about 2 mg/kg. In embodiments, the anti-CD84 antibody is administered at an amount from about 0.5 mg/kg to about 1.5 mg/kg. In embodiments, the anti-CD84 antibody is administered at an amount from about 0.5 mg/kg to about 1 mg/kg.

In embodiments, the anti-CD84 antibody is administered at an amount from 0.5 mg/kg to 19.5 mg/kg. In embodiments, the anti-CD84 antibody is administered at an amount from 0.5 mg/kg to 19 mg/kg. In embodiments, the anti-CD84 antibody is administered at an amount from 0.5 mg/kg to 18.5 mg/kg. In embodiments, the anti-CD84 antibody is administered at an amount from 0.5 mg/kg to 18 mg/kg. In embodiments, the anti-CD84 antibody is administered at an amount from 0.5 mg/kg to 17.5 mg/kg. In embodiments, the anti-CD84 antibody is administered at an amount from 0.5 mg/kg to 17 mg/kg. In embodiments, the anti-CD84 antibody is administered at an amount from 0.5 mg/kg to 16.5 mg/kg. In embodiments, the anti-CD84 antibody is administered at an amount from 0.5 mg/kg to 16 mg/kg. In embodiments, the anti-CD84 antibody is administered at an amount from 0.5 mg/kg to 15.5 mg/kg. In embodiments, the anti-CD84 antibody is administered at an amount from 0.5 mg/kg to 15 mg/kg. In embodiments, the anti-CD84 antibody is administered at an amount from 0.5 mg/kg to 14.5 mg/kg. In embodiments, the anti-CD84 antibody is administered at an amount from 0.5 mg/kg to 14 mg/kg. In embodiments, the anti-CD84 antibody is administered at an amount from 0.5 mg/kg to 13.5 mg/kg. In embodiments, the anti-CD84 antibody is administered at an amount from 0.5 mg/kg to 13 mg/kg. In embodiments, the anti-CD84 antibody is administered at an amount from 0.5 mg/kg to 12.5 mg/kg. In embodiments, the anti-CD84 antibody is administered at an amount from 0.5 mg/kg to 12 mg/kg. In embodiments, the anti-CD84 antibody is administered at an amount from 0.5 mg/kg to 11.5 mg/kg. In embodiments, the anti-CD84 antibody is administered at an amount from 0.5 mg/kg to 11 mg/kg. In embodiments, the anti-CD84 antibody is administered at an amount from 0.5 mg/kg to 10.5 mg/kg. In embodiments, the anti-CD84 antibody is administered at an amount from 0.5 mg/kg to 10 mg/kg.

In embodiments, the anti-CD84 antibody is administered at an amount from 0.5 mg/kg to 9.5 mg/kg. In embodiments, the anti-CD84 antibody is administered at an amount from 0.5 mg/kg to 9 mg/kg. In embodiments, the anti-CD84 antibody is administered at an amount from 0.5 mg/kg to 8.5 mg/kg. In embodiments, the anti-CD84 antibody is administered at an amount from 0.5 mg/kg to 8 mg/kg. In embodiments, the anti-CD84 antibody is administered at an amount from 0.5 mg/kg to 7.5 mg/kg. In embodiments, the anti-CD84 antibody is administered at an amount from 0.5 mg/kg to 7 mg/kg. In embodiments, the anti-CD84 antibody is administered at an amount from 0.5 mg/kg to 6.5 mg/kg. In embodiments, the anti-CD84 antibody is administered at an amount from 0.5 mg/kg to 6 mg/kg. In embodiments, the anti-CD84 antibody is administered at an amount from 0.5 mg/kg to 5.5 mg/kg. In embodiments, the anti-CD84 antibody is administered at an amount from 0.5 mg/kg to 5 mg/kg. In embodiments, the anti-CD84 antibody is administered at an amount from 0.5 mg/kg to 4.5 mg/kg. In embodiments, the anti-CD84 antibody is administered at an amount from 0.5 mg/kg to 4 mg/kg. In embodiments, the anti-CD84 antibody is administered at an amount from 0.5 mg/kg to 3.5 mg/kg. In embodiments, the anti-CD84 antibody is administered at an amount from 0.5 mg/kg to 3 mg/kg. In embodiments, the anti-CD84 antibody is administered at an amount from 0.5 mg/kg to 2.5 mg/kg. In embodiments, the anti-CD84 antibody is administered at an amount from 0.5 mg/kg to 2 mg/kg. In embodiments, the anti-CD84 antibody is administered at an amount from 0.5 mg/kg to 1.5 mg/kg. In embodiments, the anti-CD84 antibody is administered at an amount from 0.5 mg/kg to 1 mg/kg.

### EXAMPLES

### Example 1: Production of Recombinant Human CD84 Proteins

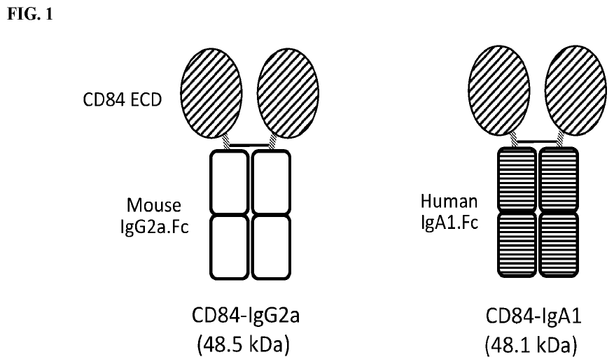

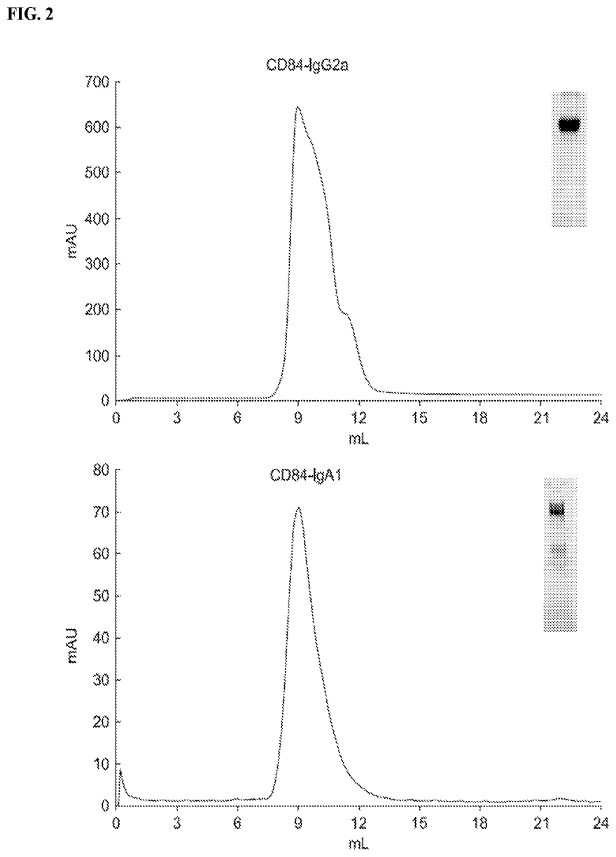

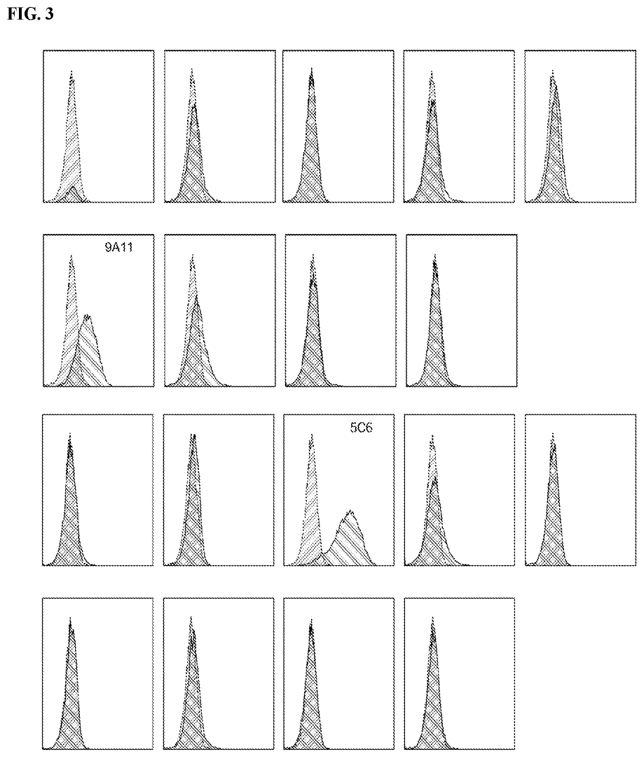

To construct recombinant CD84 proteins, the extracellular domain (ECD) of human CD84 was fused with mouse IgG2a. Fc or human IgA1.Fc, respectively (FIG. 1). Recombinant DNAs encoding these two proteins were synthesized and cloned into mammalian expression vectors by the manufacturer (Twist Bioscience). The DNAs were prepared using a ZymoPURE™ II Plasmid Maxiprep Kit (Zymo Research). The CD84 fusion proteins were produced using an ExpiCHO expression system (Thermo Fisher Scientific). The procedures were followed according to the manufacturer's manual. In brief, CHO cells were seeded at 3-4×10⁶ cells/mL in fresh ExpiCHO expression medium 24 h before DNA transfection. On the day of transfection cells were adjusted to 6×10⁶ cells/mL with fresh medium. For a 100-mL transfection, 80 mg of DNA was diluted in 4 mL of OptiPRO™ SFM and then mixed with 320 mL of ExpiFectamine™ diluted in 3.7 mL OptiPRO™ SFM. The mixture was slowly added into the cell culture with gentle swirling. Cells were then cultured at 37° C. After 16-22 h 6 ml of ExpiCHO™ Enhancer and 24 mL of ExpiCHO™ Feed were added into the cell culture, and cells were kept culturing at 32° C. for 10 days. Culture supernatants were harvested by centrifugation at 4000×g for 30 minutes and passed through 0.22-mm filters for protein purification. Protein A resins (GE Healthcare) and CaptureSelect™ IgA Affinity Matrix (Thermo Fisher Scientific) were used to purify CD84-IgG2a and CD84-IgA1, respectively. Purification procedures were followed according to manufacturer's manuals. Purified proteins were run using reduced SDS-PAGE with Mini-PROTEAN® TGX Stain-Free™ Precast Gels (Bio-Rad) and analyzed using a Superdex 200 Increase 10/300 GL column (GE Healthcare) with PBS (FIG. 2).

### Example 2: Preparations of Anti-Cd84 Hybridomas

All animal experiments were conducted under the approval of Institutional Animal Care and Use Committee of City of Hope (IUCAC #19070 and #19023). For mouse immunization, recombinant CD84-IgG2a proteins were emulsified with complete Freund's adjuvants (Sigma Aldrich) and subcutaneously injected into 10 Balb/c mice (The Jackson Laboratory). Fifty micrograms of proteins were injected for each mouse. After three weeks, mice received two subcutaneous injections with 50 mg of CD84-IgG2a emulsified with incomplete Freund's adjuvants (Sigma Aldrich) in a two-week interval. Three days before spleen harvests, 10 mg of CD84-IgG2a were injected into mice via tail veins. Spleen cells were harvested for fusion with mouse myeloma cell line FO (ATCC) using PEG 1500 (Roche). The cell fusion procedures were followed according to the manufacturer's manual. After fusion, cells were selected in complete DMEM medium containing hypoxanthine/aminopterin/thymidine (Thermo Fisher Scientific) and % UltraCruz® Hybridoma Cloning Supplement (Santa Cruz) for 10-12 days. Hybridoma culture supernatants were screened for reacting to CD84-IgA1 proteins using ELISAs. For screening with ELISA 50 mL of CD84-IgA1 diluted in carbonate/bicarbonate buffer, pH 9.6 at the concentration of 1 mg/mL were added into micro-wells and incubated at 4° C. overnight. Wells were washed with PBS containing 0.1% Tween 20 (PBST) three times and blocked with 200 mL of PBS containing 1% bovine serum albumin. After incubation at room temperature for 1 h and wash with PBST three times, 50 mL of culture supernatants were added into wells and incubated at room temperature for 1 hr. After wash, 50 mL of 1:10,000 diluted goat anti-mouse IgG.Fc-HRP (Jackson ImmunoResearch) were added into wells and incubated at room temperature for 1 hr. After wash six times, 50 mL of TMB substrates (Thermo Fisher Scientific) were added into wells for color development. The reactions were stopped by adding 50 mL of 1N HCl. Wells were read at optical density 450 nm with a Synergy 4 microplate reader (BioTek). In ELISA screening results 71 hybridoma clones showed strong binding signals with CD84-IgA1 proteins.

### Example 3: Flow Cytometry of Anti-CD84 Hybridoma Clones

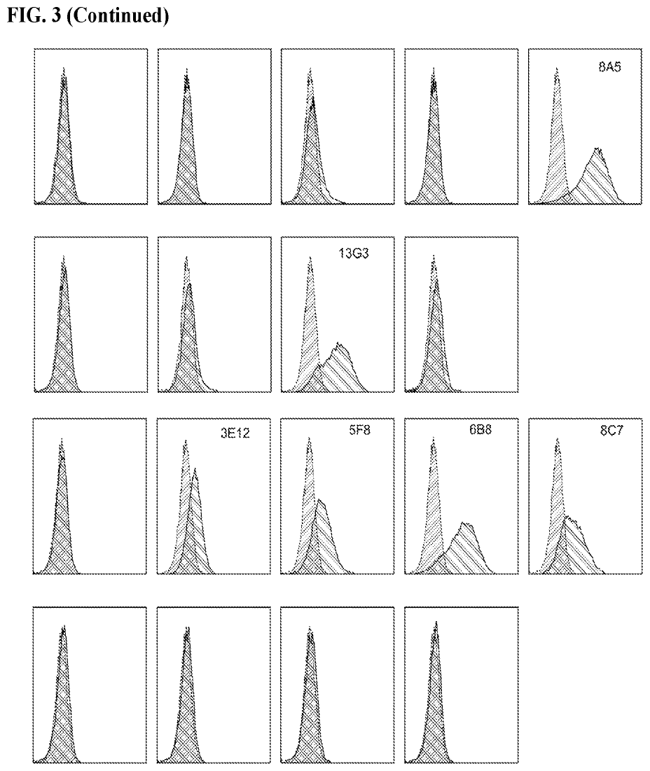

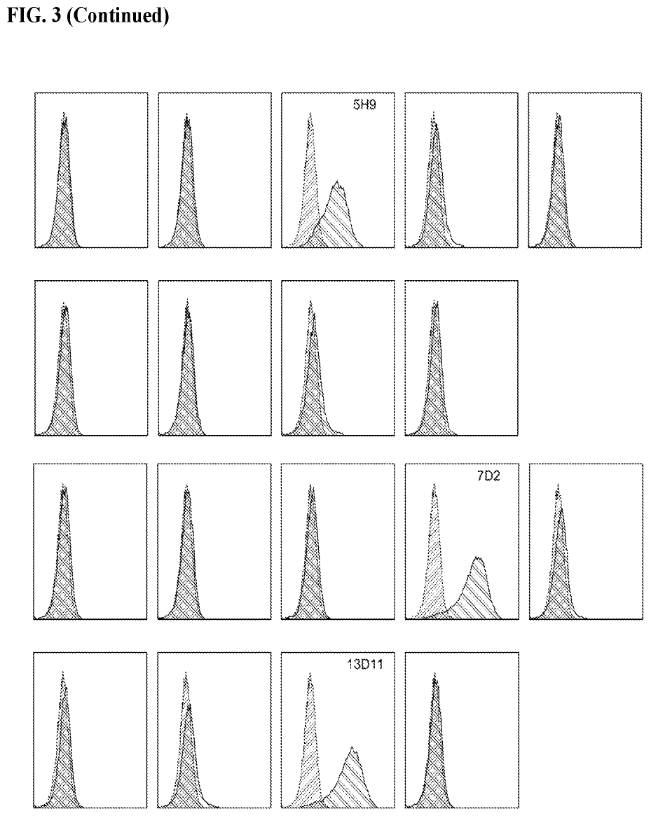

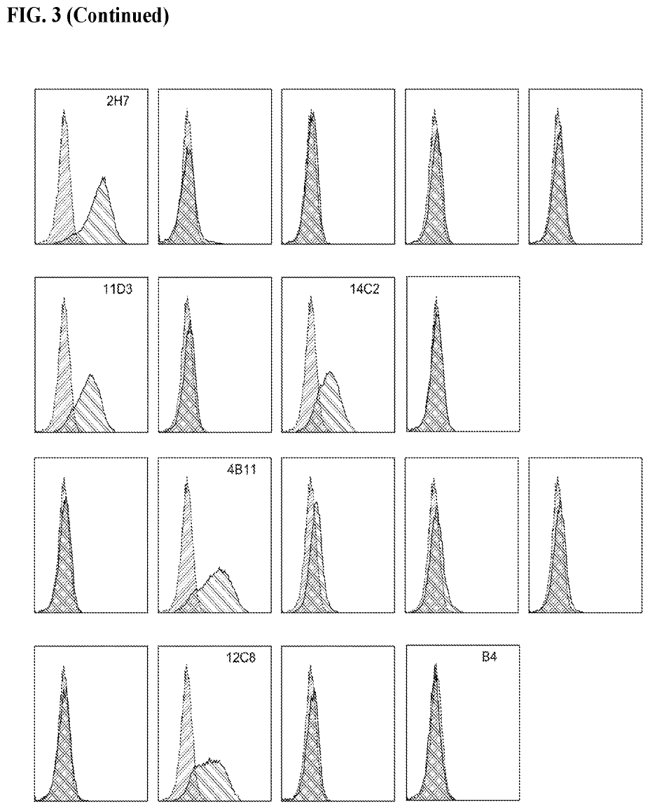

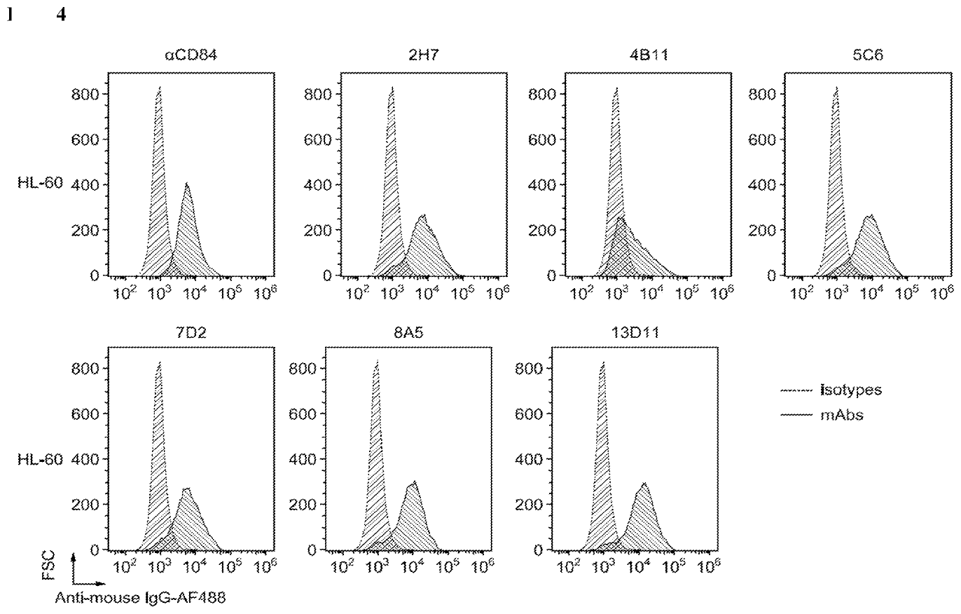

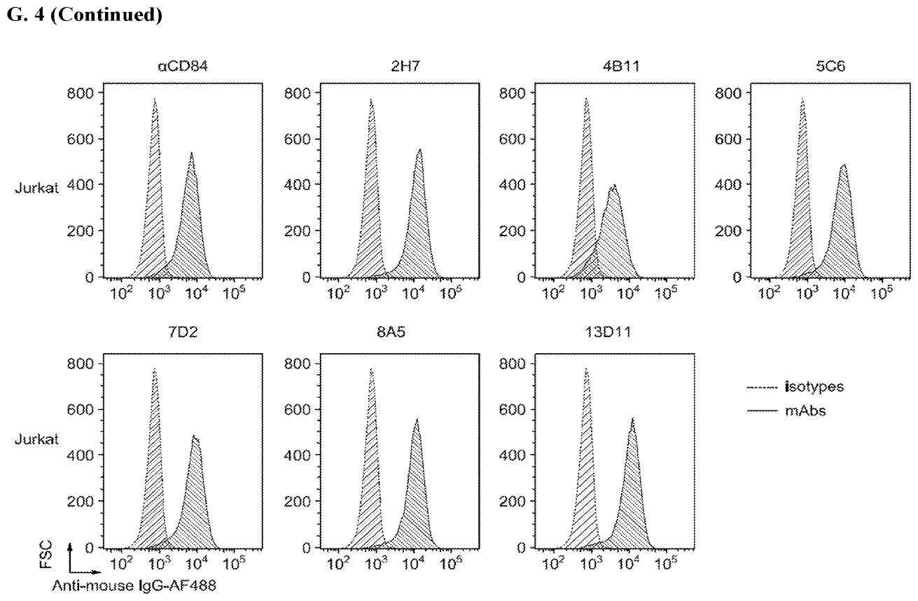

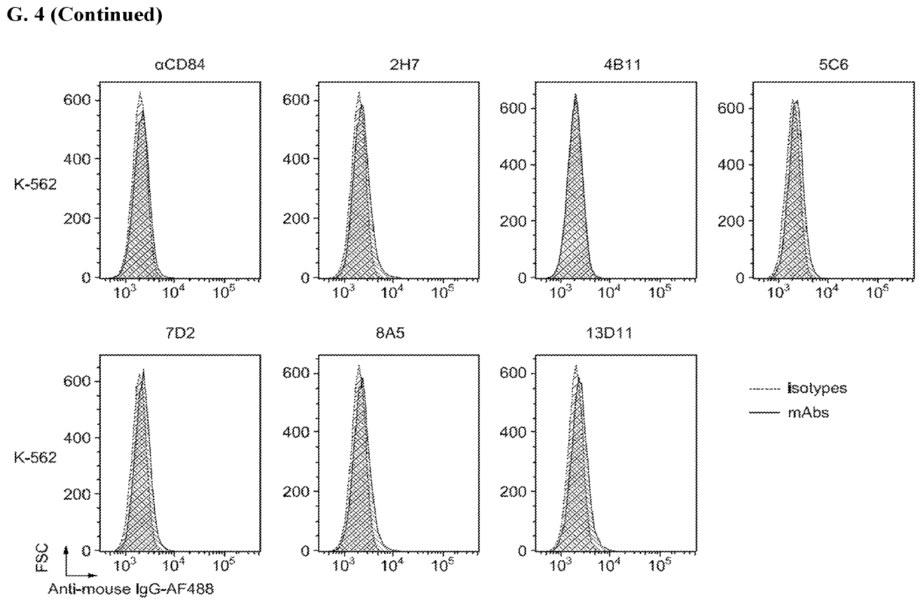

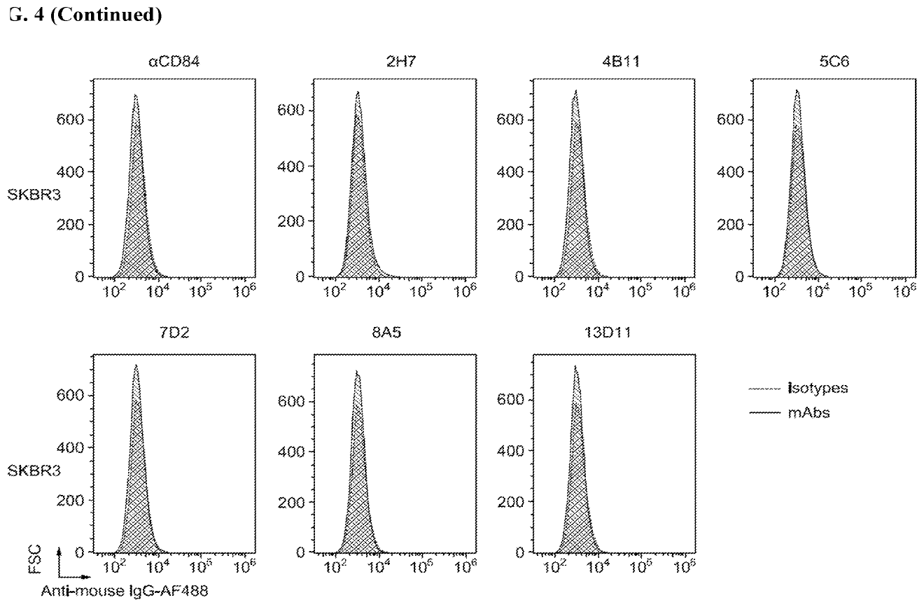

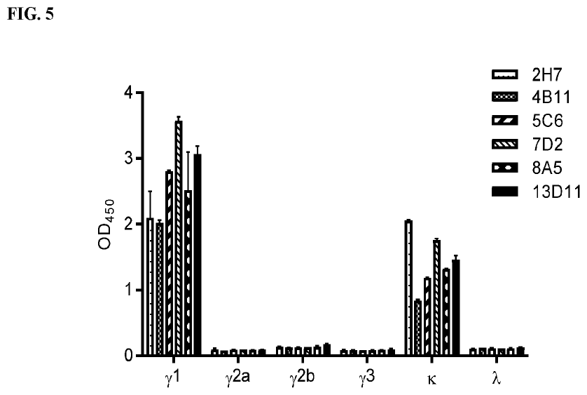

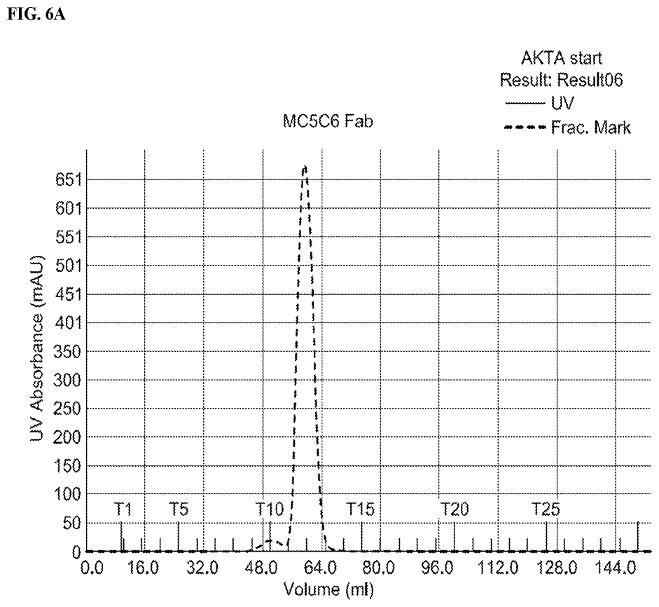

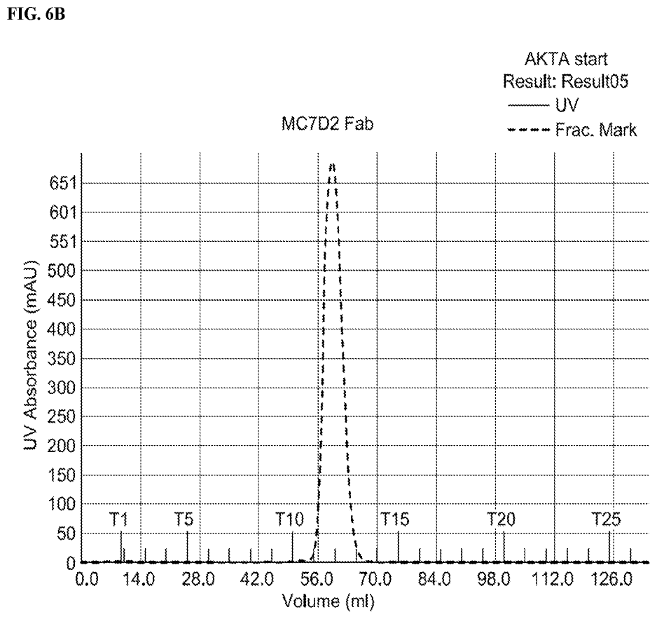

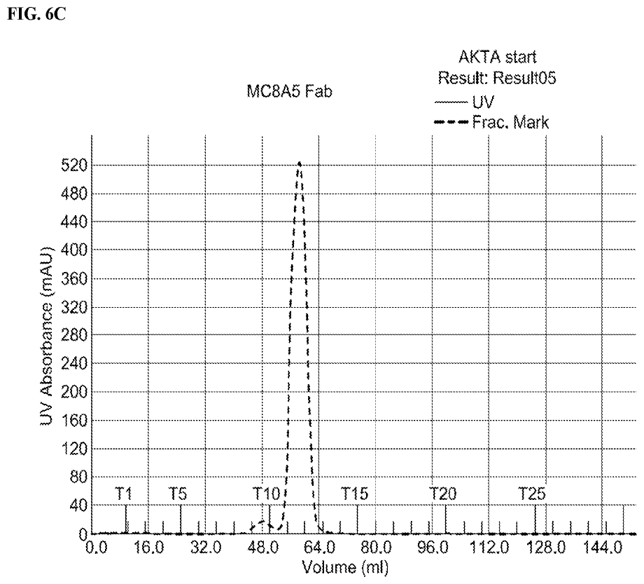

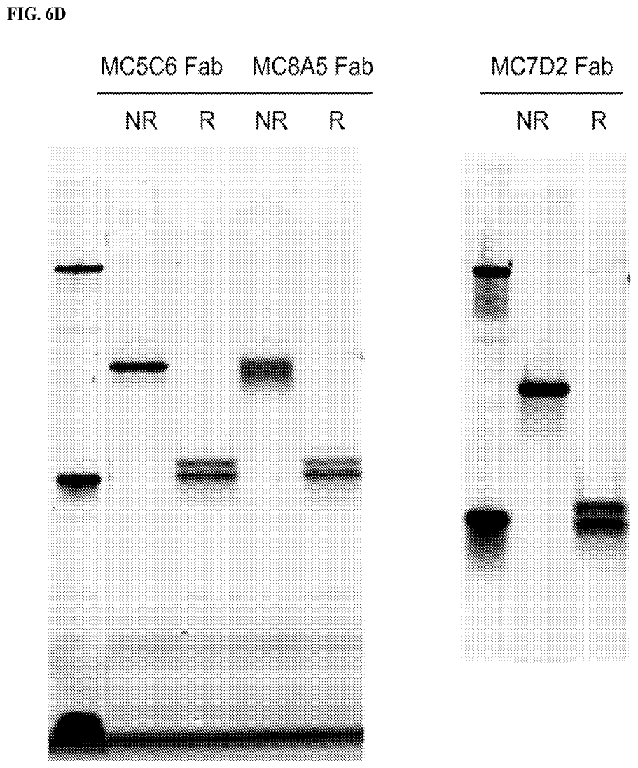

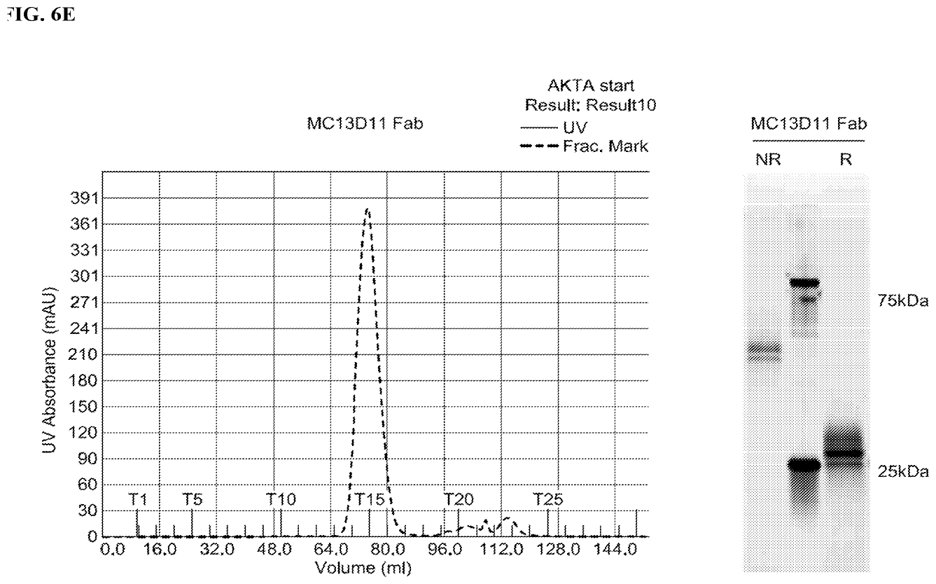

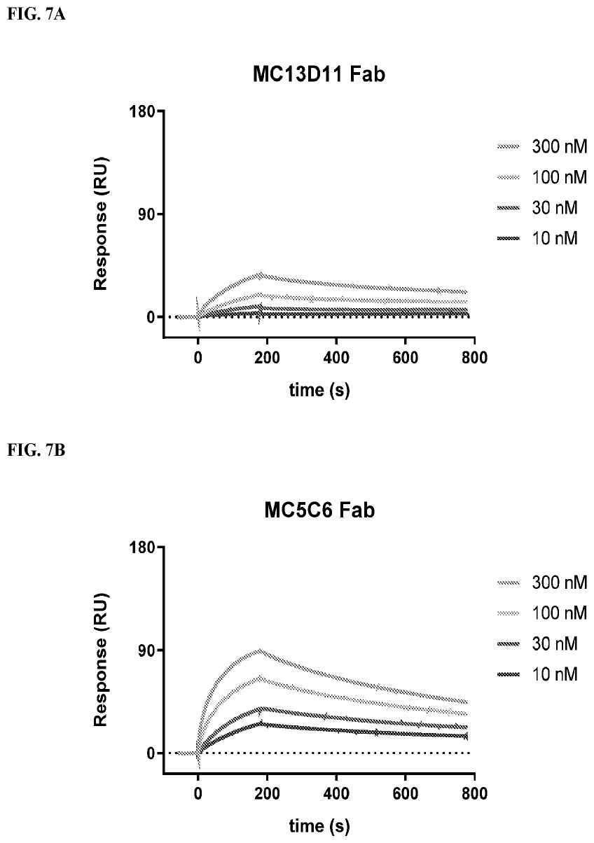

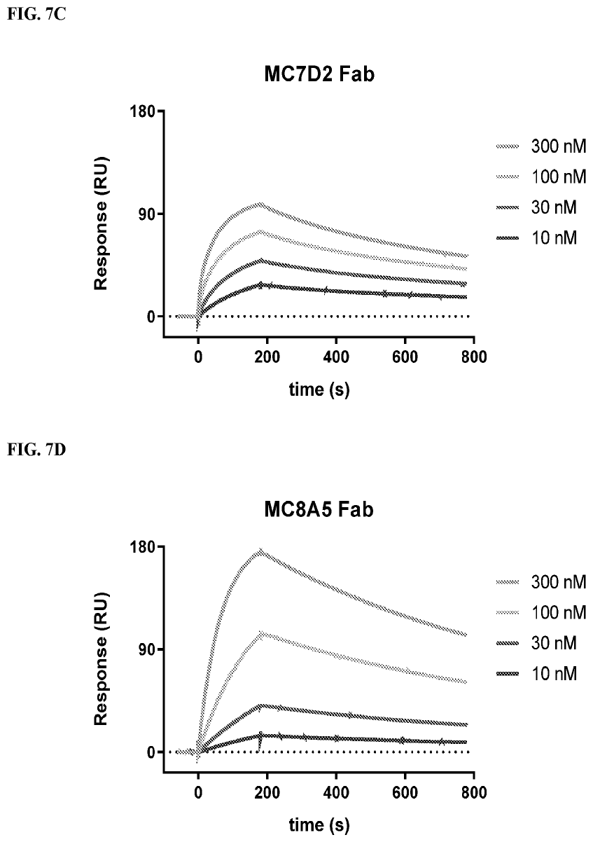

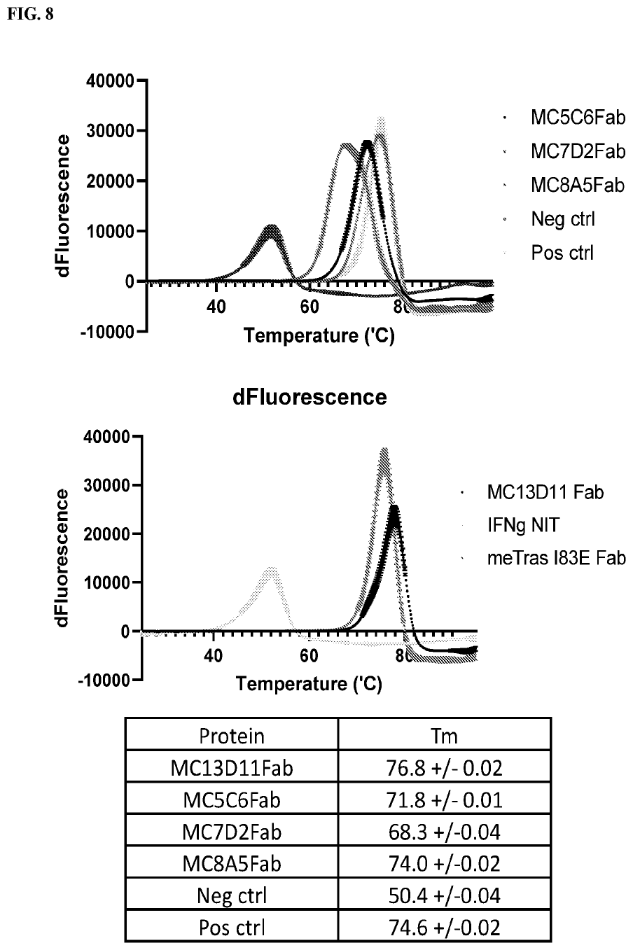

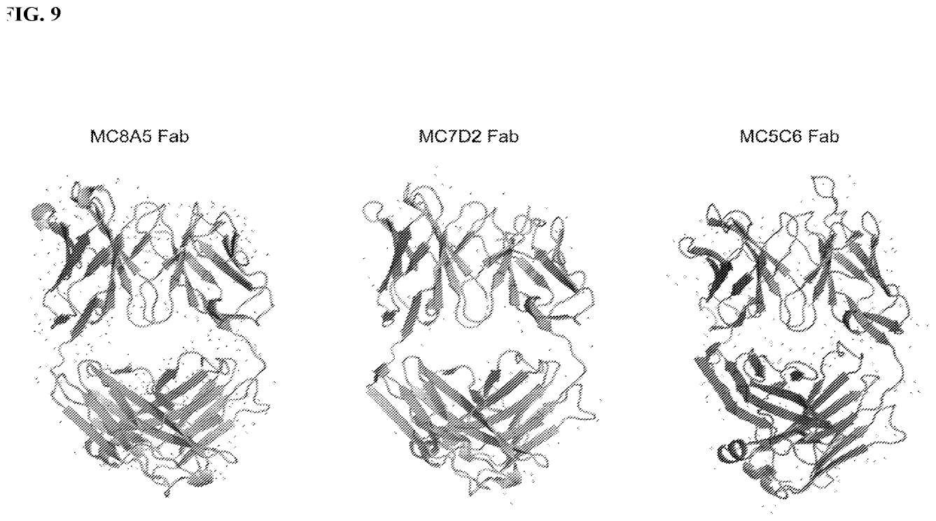

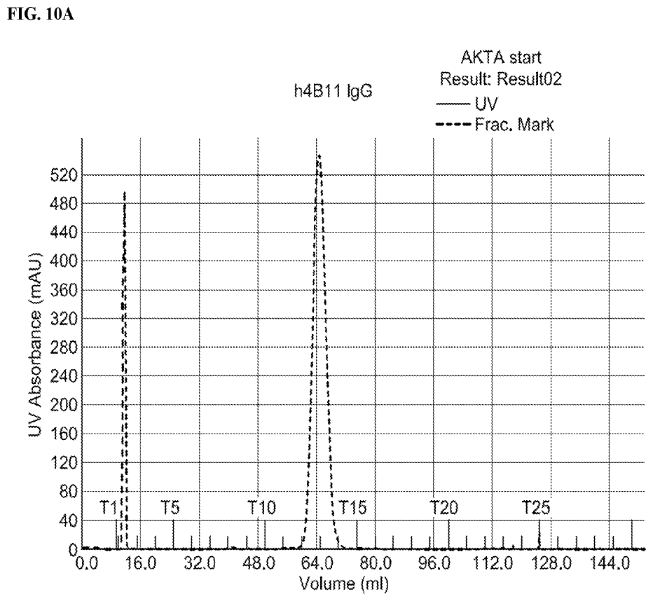

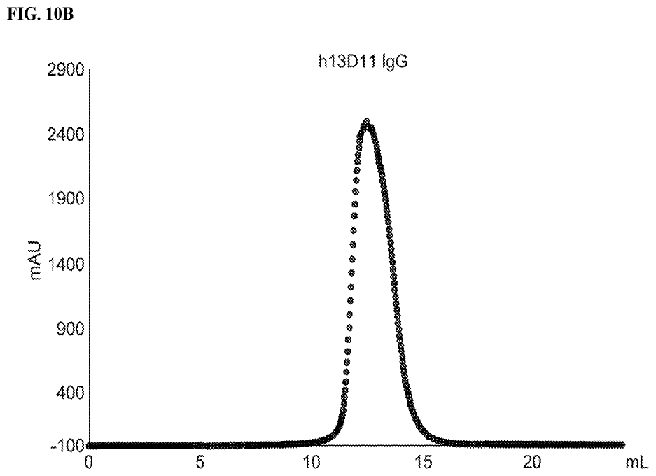

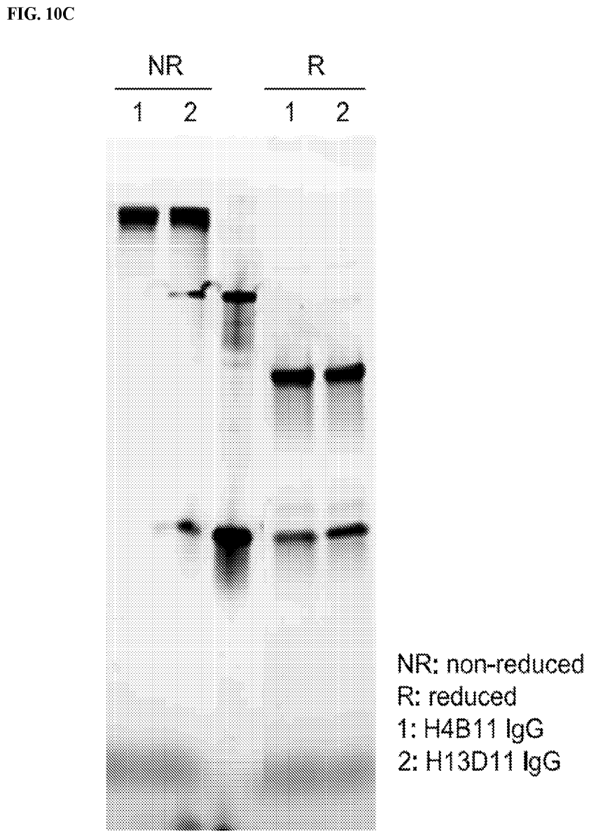

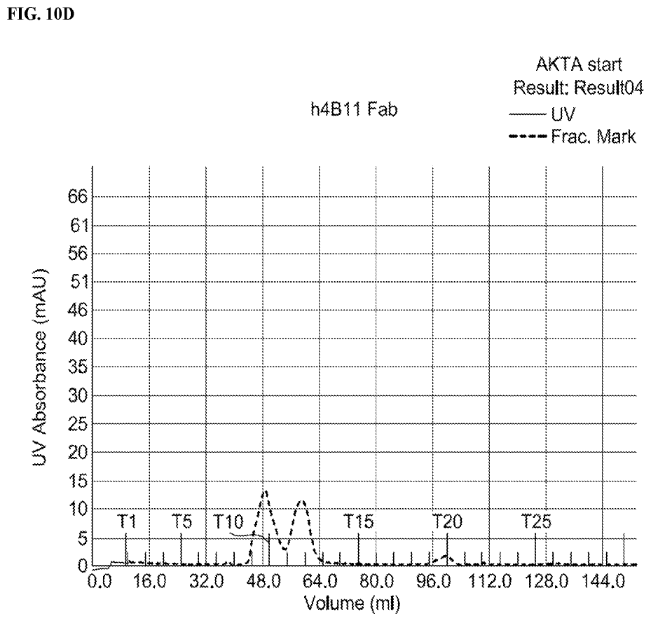

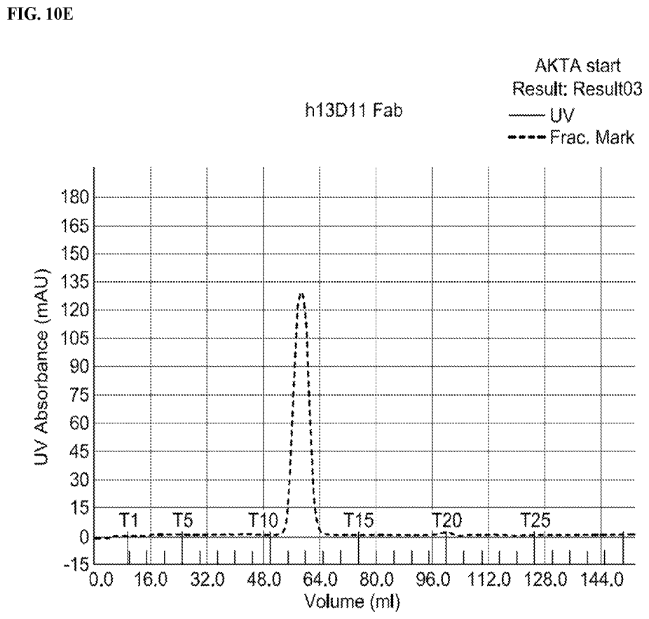

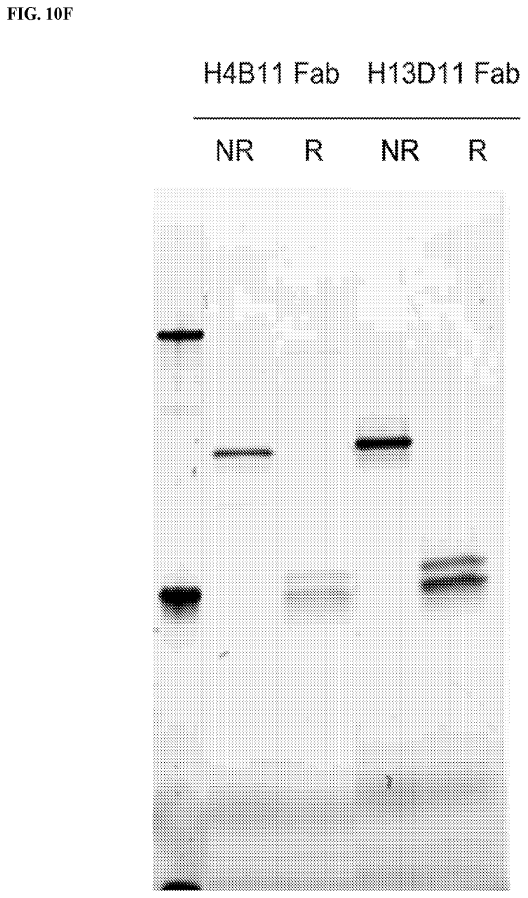

Hybridoma clones selected from ELISA screening were subjected to flow cytometric analyses with NSO, a mouse myeloma cell line (ECACC), expressing human CD84 via lentiviral transduction. Half a million of transduced cells were incubated with 100 mL of hybridoma culture supernatants on ice for 30 min and washed with cold PBS containing 1% fetal bovine serum and 0.1% sodium azide. Cells were then incubated with 100 mL of 1:400 diluted Alexa Fluor® 488 AffiniPure goat anti-mouse IgG (Jackson ImmunoResearch) on ice for 30 min and washed for analyses with an Accuri C6 flow cytometer (BD). The results showed that 16 clones could bind to human CD84 on the cell surface (FIG. 3). Six clones with strong staining signals, which were 2H7, 4B11, 5C6, 7D2, 8A5, and 13D11 (Table 1), were further tested for their binding to human myeloma cell line HL-60, T cell line Jurkat, lymphoma cell line K-562, and mammary gland cell line SKBR3 (ATCC). A commercial mouse anti-human CD84 monoclonal antibody (aCD84, BioLegend) was used as a positive control. The results showed that, consistent with the positive control, these anti-CD84 antibodies specifically bind to CD84-expressing cells without reacting to CD84-negative cells (FIG. 4).

TABLE 1

| | Geometric |
|---|---|
| Clones | Mean |
| 2H7 | 1546 |
| 3E12 | 208 |
| 4B11 | 1098 |
| 5C6 | 1687 |
| 5H9 | 698 |
| 5F8 | 276 |
| 6B8 | 935 |
| 7D2 | 2255 |
| 8A5 | 1812 |
| 8C7 | 343 |
| 9A11 | 351 |
| 11D3 | 673 |
| 12C8 | 619 |
| 13D11 | 2125 |
| 13G3 | 739 |
| 14C2 | 423 |
| Ctrl | 88 |

### Example 4: Isotyping and Cloning of VH/VL of Anti-CD84 Antibodies

To determine the Ig isotypes of mouse antibodies, CD84-IgA1 proteins were used as antigens immobilized for incubation with hybridoma culture supernatants in ELISAs. HRP-conjugated goat anti-mouse Igg1, Igg2a, Igg2b, Igg3, Igk, and Ig1 (SouthernBiotech) were used to detect antibody isotypes and to develop colors with TMB substrates. All of these six antibodies were of g1 and kappa isotypes (FIG. 5). To clone VH/VL sequences from hybridomas, mRNAs were extracted using a Quick-RNA Microprep kit (Zymo Research). First-strand cDNAs were synthesized using a SuperScript III First-Strand Synthesis System (Thermo Fisher Scientific). The VH and VL fragments were amplified by PCRs using a Mouse Ig-Primer Set (Millipore Sigma) and OneTaq 2× Master Mix (NEB). Amplified DNA fragments were purified using a DNA Clean-up kit (Zymo Research) and ligated into pGEM-T vectors (Promega) for sequencing.

### Example 5: CD84 is Overexpressed in AML and is Required for the Growth of Human AML Cells

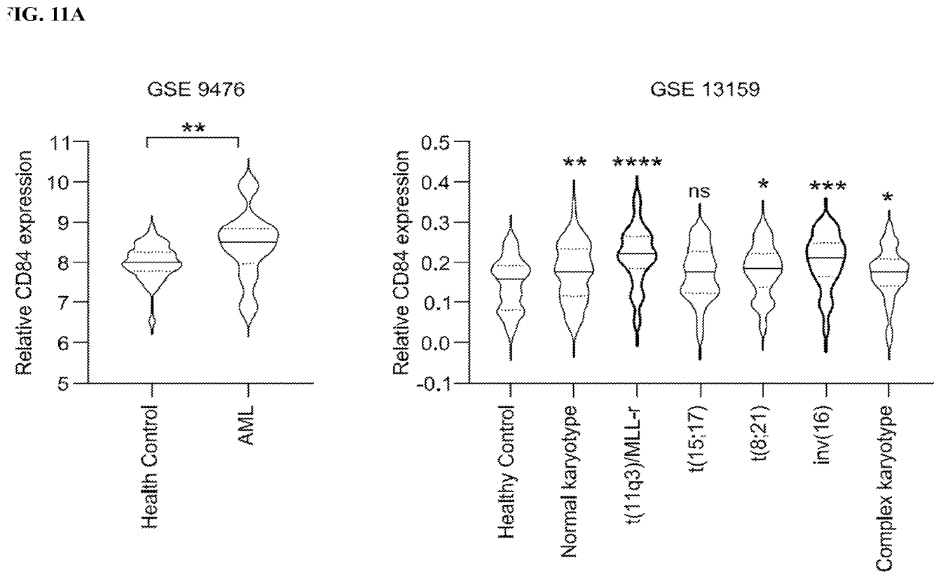

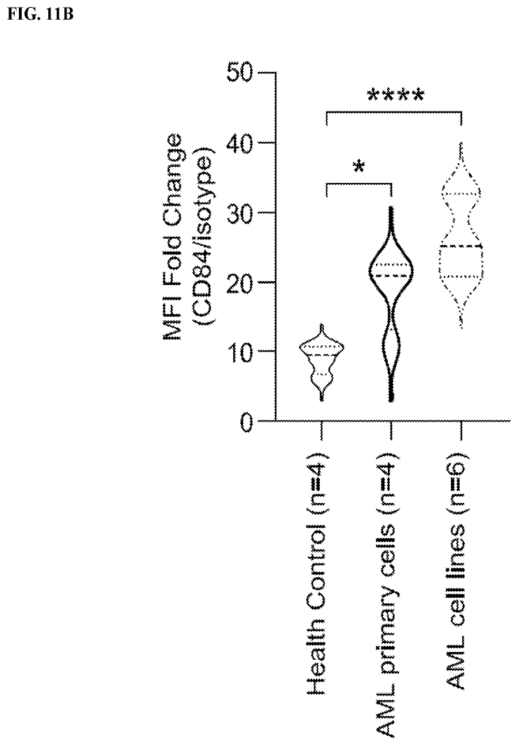

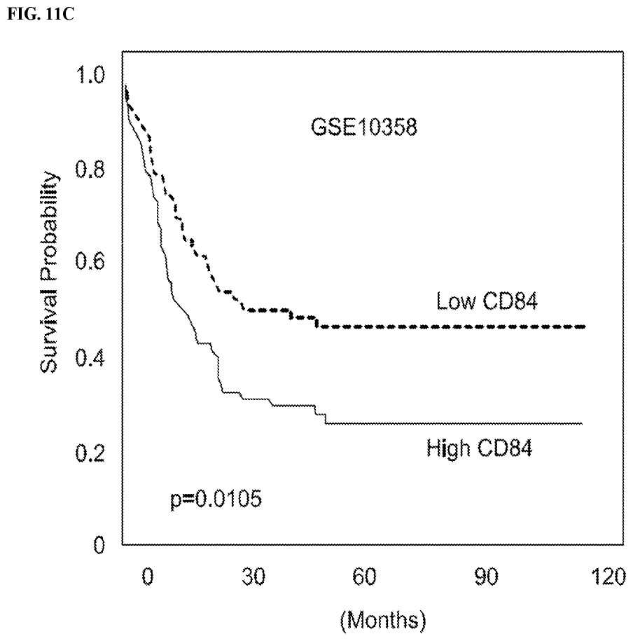

Based on the gene expression profiling datasets that including large cohorts of AML patients (GSE 9476 and GSE13159), we observed that bone marrow mononuclear cells of AML specimens showed significantly increased CD84 mRNA levels compare to those of normal healthy donor (FIG. 11A, left panel). Notably, relative to other AML subtypes, the myelomonocytic and monoblastic leukemia patients with t (11q23) and inv (16) abnormalities exhibited even higher CD84 expression (FIG. 11A, highlighted in right panel). We further confirmed that surfaced CD84 was overexpressed in AML cell lines (n=6) and primary cells (n=4) compared with that seen in healthy donor (FIG. 11B). Moreover, we also found that higher CD84 expression is associated with shorter overall survival (OS) in AML patients (GSE10358) (FIG. 11C).

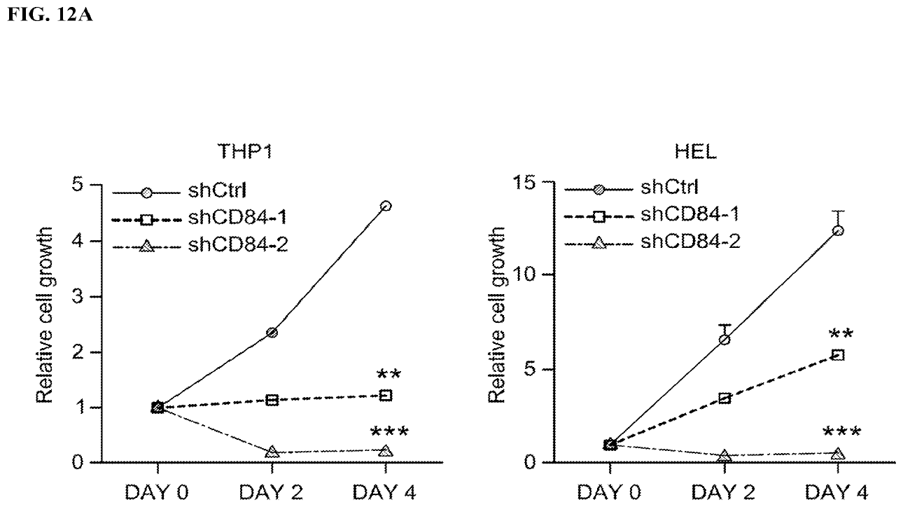

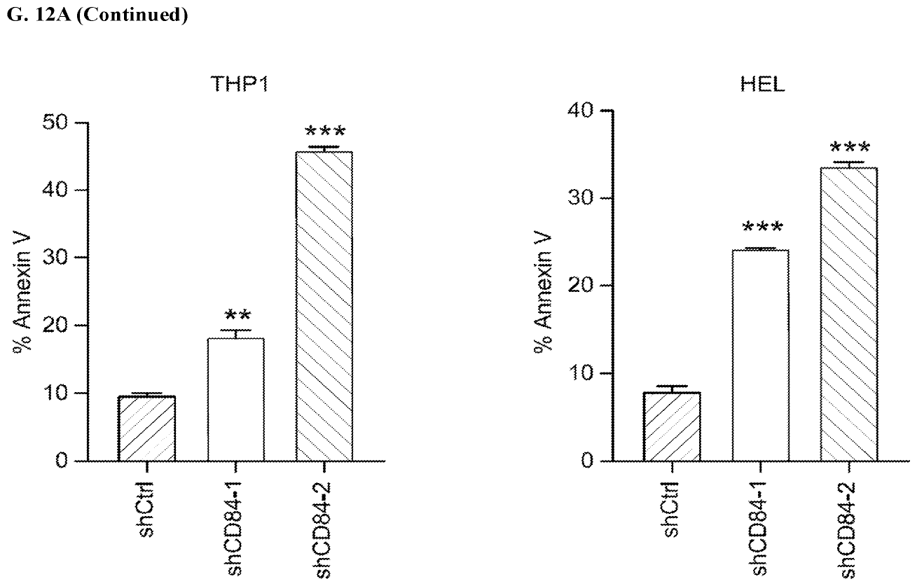

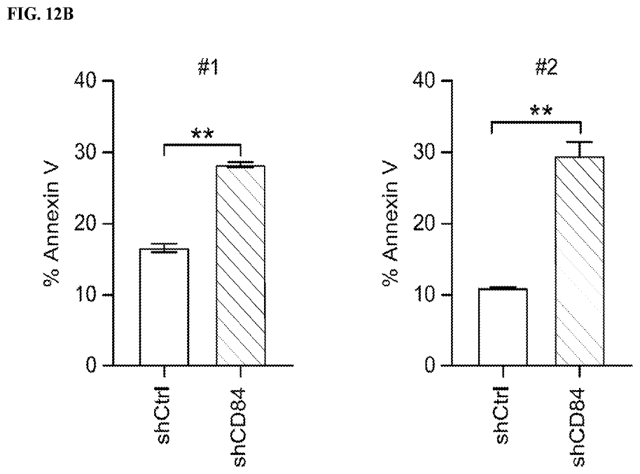

To investigate the role of CD84 in AML, both gain- and loss-of-function studies are conducted. CD84 knockdown by short hairpin RNA (shRNAs) caused a substantial inhibition on cell growth and apoptosis (FIG. 12A, left and right panel, respectively) in AML cell lines. In primary AML cells, we found CD84 knockdown dramatically induced cell apoptosis and inhibited colony number (FIG. 12B, left and right panel, respectively).

Furthermore, we knockdown CD84 in luciferase-expressing THP1 cells, and then transplanted selected cells into immunodeficient NSG mice. Attenuated tumor burden (FIG. 12C, left panel) as well as prolonged survival (FIG. 12C, right panel) were observed in the mice transplanted with CD84 knockdown cells, relative to control mice.

### Example 6: CD84 Expression Depletion Impairs AML Cell Repopulation and AML Maintenance

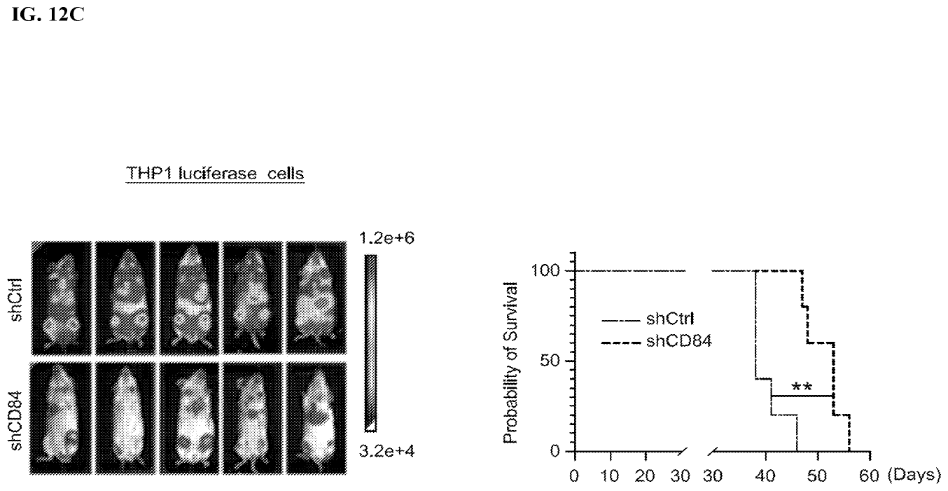

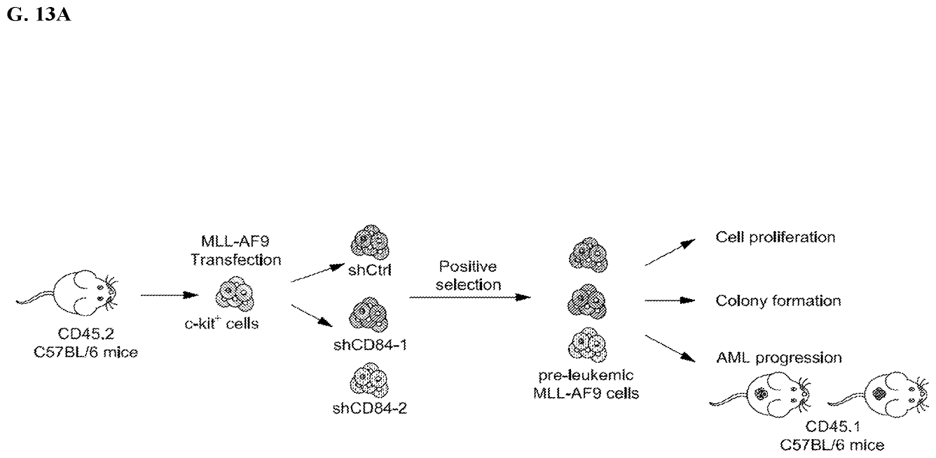

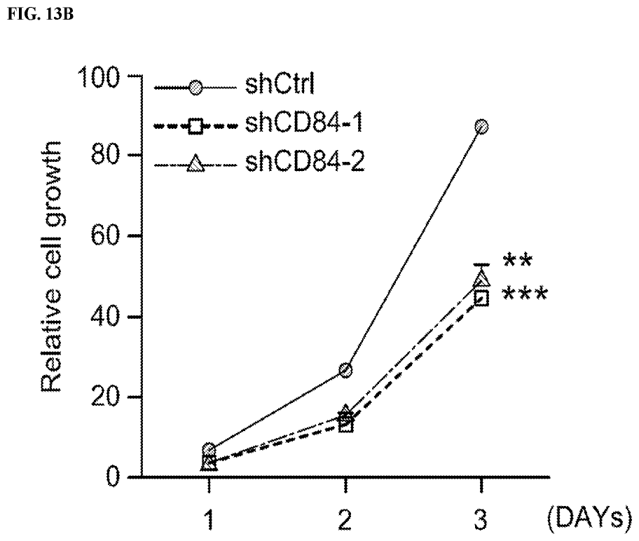

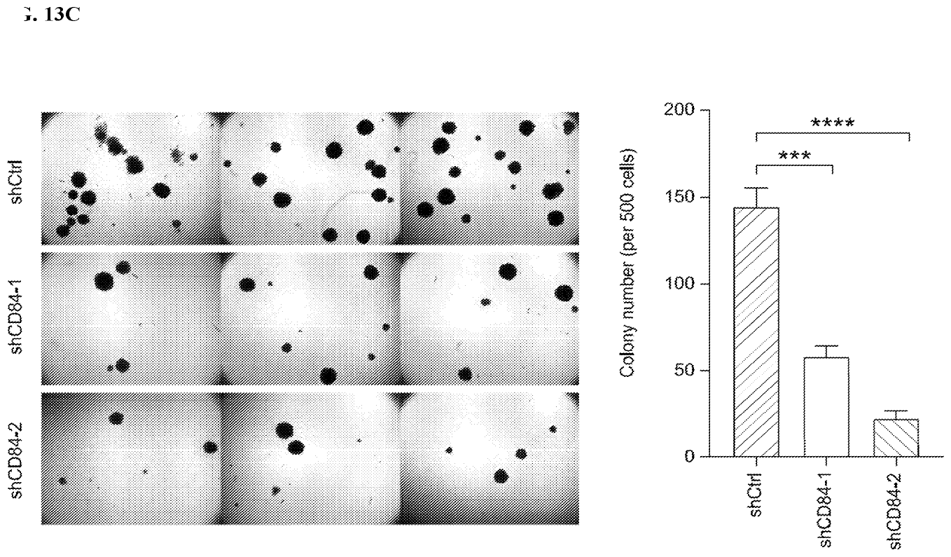

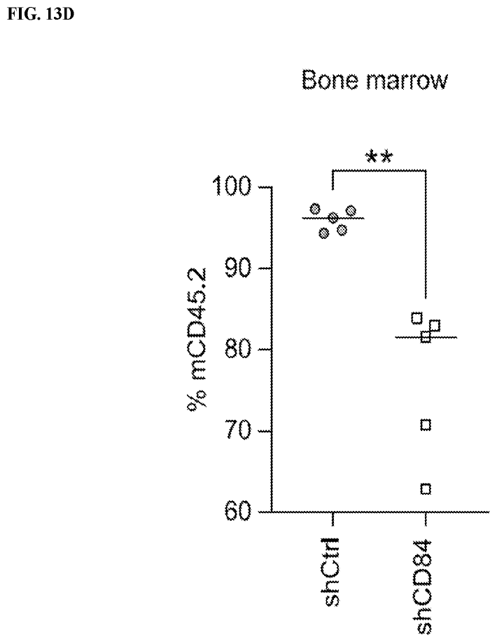

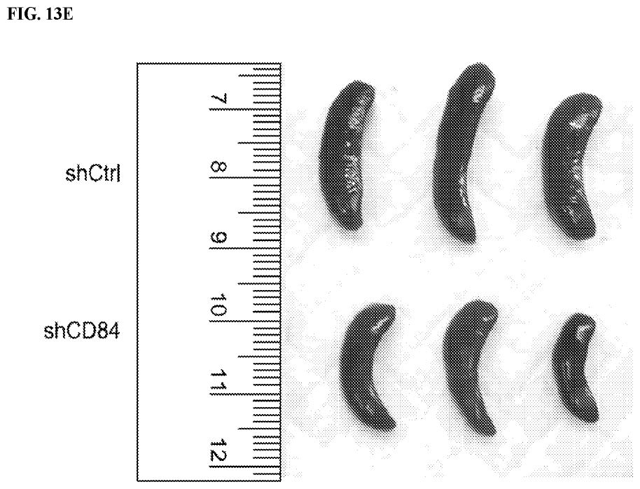

To understand the functional involvement of CD84 in leukemogenesis, we used the lentiviral vector-based shRNA system to knockdown the expression of CD84 (shCD84-1 and shCD84-2) in murine MLL-AF9-HSPC (hematopoietic stem/progenitor cell) pre-LSCs (Leukemia Stem Cells), which were generated by transducing mouse HSPC with lentivirus encoding the MLL-AF9 fusion oncogene. As shown in FIG. 13A, MLL-AF9-HSPC pre-LSCs transduced with shCD84 (CD84 KD) had slower cell growth (FIG. 13B). As expected, colony formation assay (CFA) indicated stable CD84 KD with both shRNA significantly decreased the colony number in transduced MLL-AF9-HSPC cells (FIG. 13C). To evaluate the role of CD84 in leukemogenesis in vivo, we conducted mouse bone marrow transplantation assays in sublethally irradiated C57BL/6 syngeneic recipient mice. We found that mice transplanted with CD84KD MLL-AF9-HSPC cells had reduced bone marrow and splenomegaly (FIGS. 13D-13E) compared to recipient mice engrafted with MLL-AF9 cells transduced with the sh-control vector.

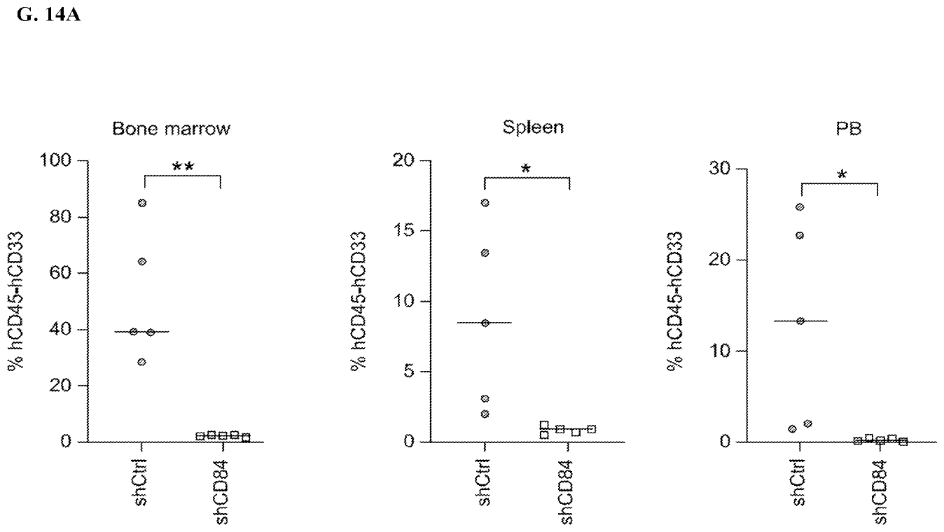

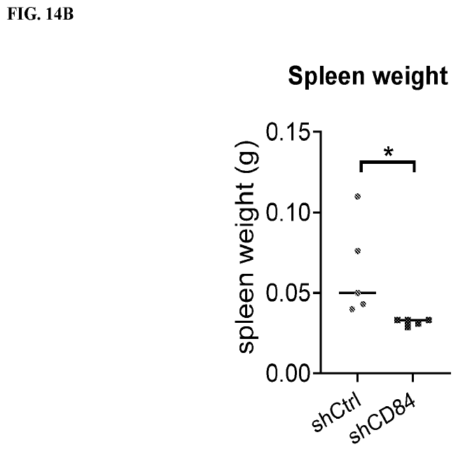

We next conducted in vivo transplantation (to evaluate the potential of CD84 in the maintenance of human AML. We transfected CD84 shRNA (sh-CD84-2) and control shRNA into AML primary patient cells. Also in this case, we observed that AML burden was significantly lower in the BM, spleen and peripheral blood (PB) of recipient mice engrafted with primary CD84 knockdown AML cells, compared with mice engrafted with shcontrol AML cells (FIG. 14A). We also found the reduced spleen weight in CD84 knockdown mice (FIG. 14B). Collectively, our data suggest that CD84 is required for the maintenance of AML.

### Example 7: CD84 is a Survival Receptor in AML and Anti-CD84 Antibodies Strongly Induce ADCC in AML Cell Lines

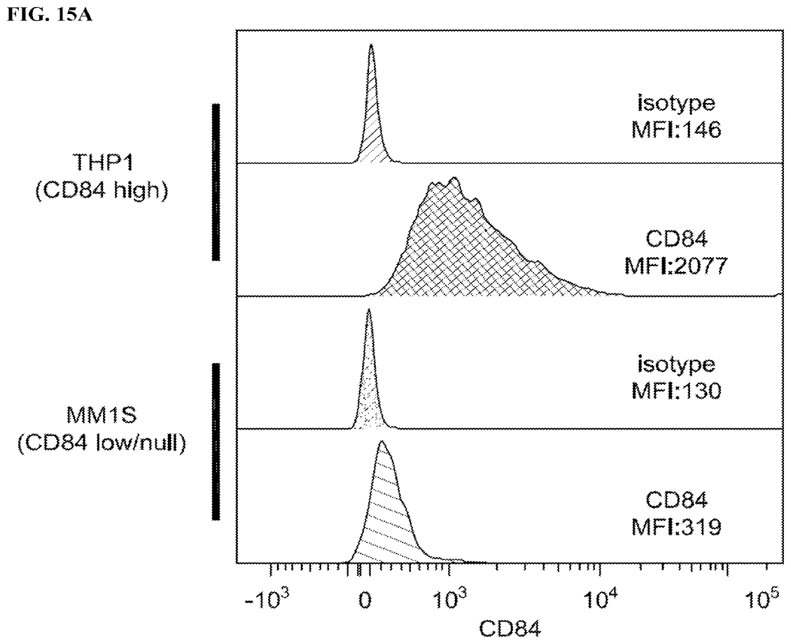

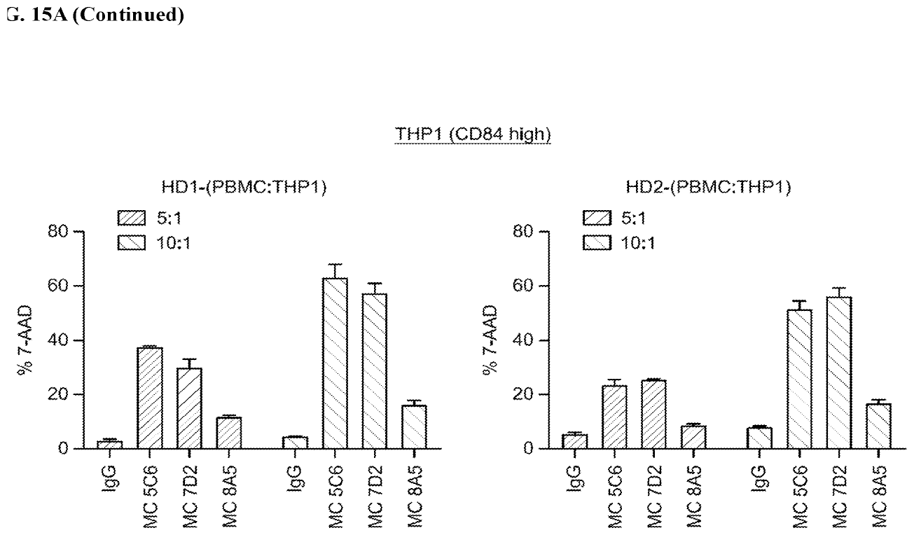

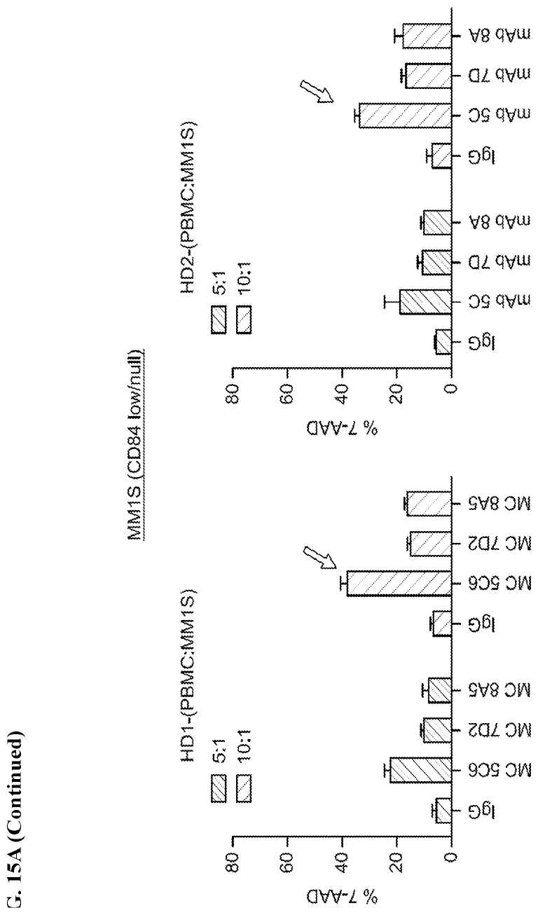

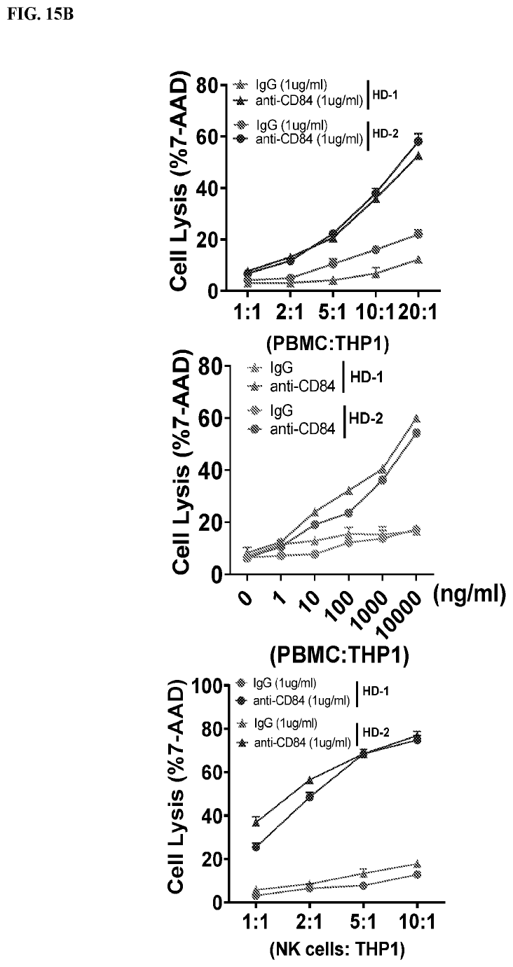

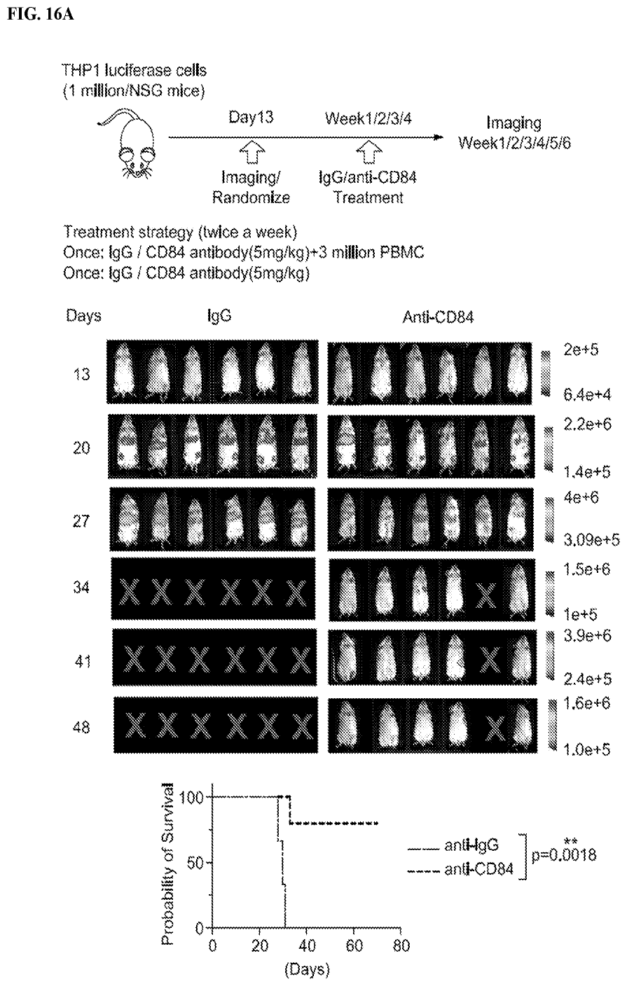

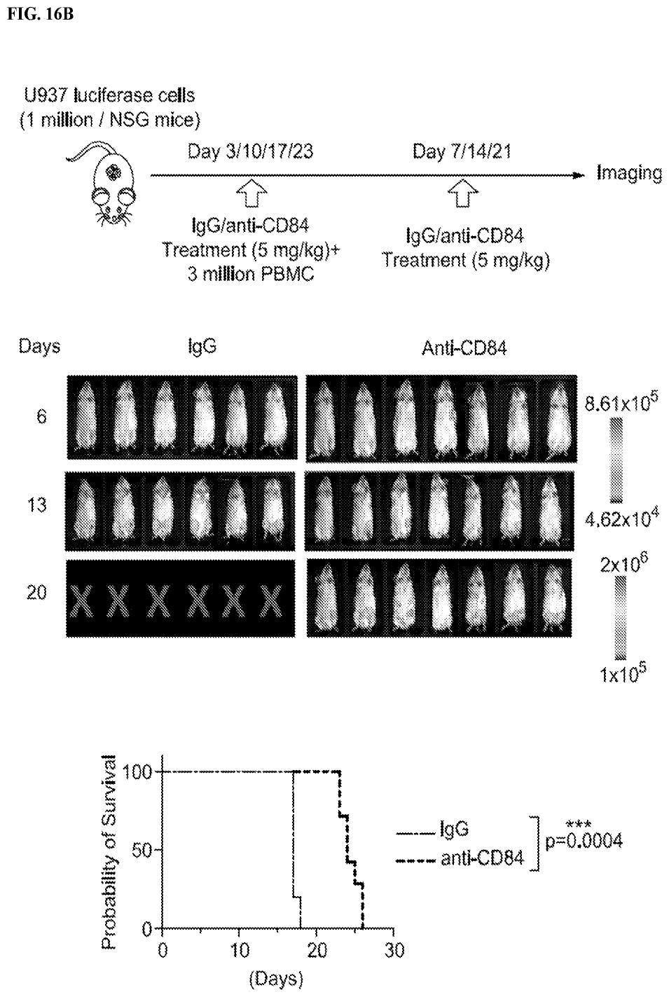

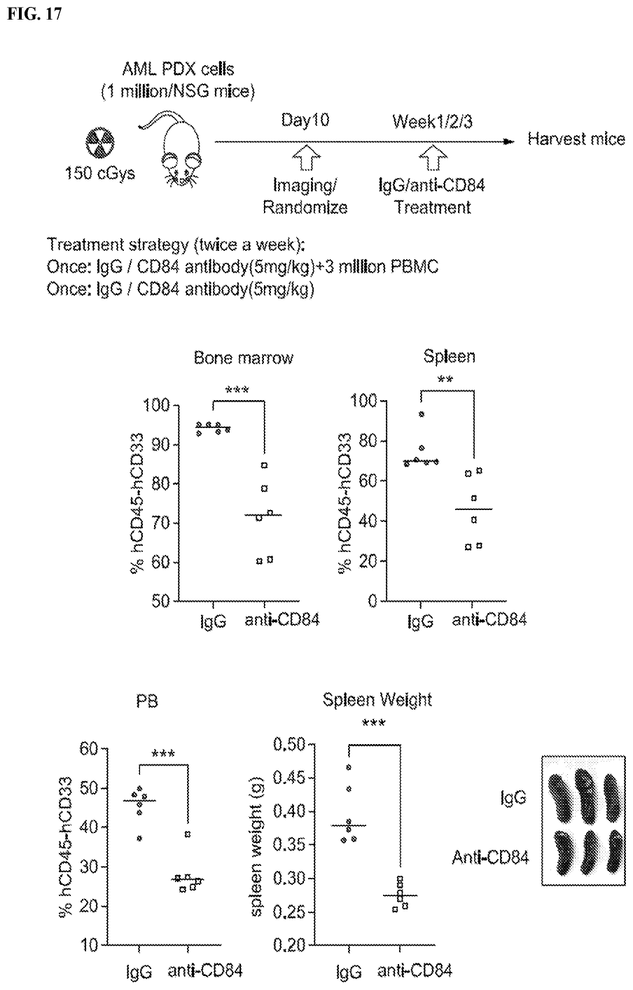

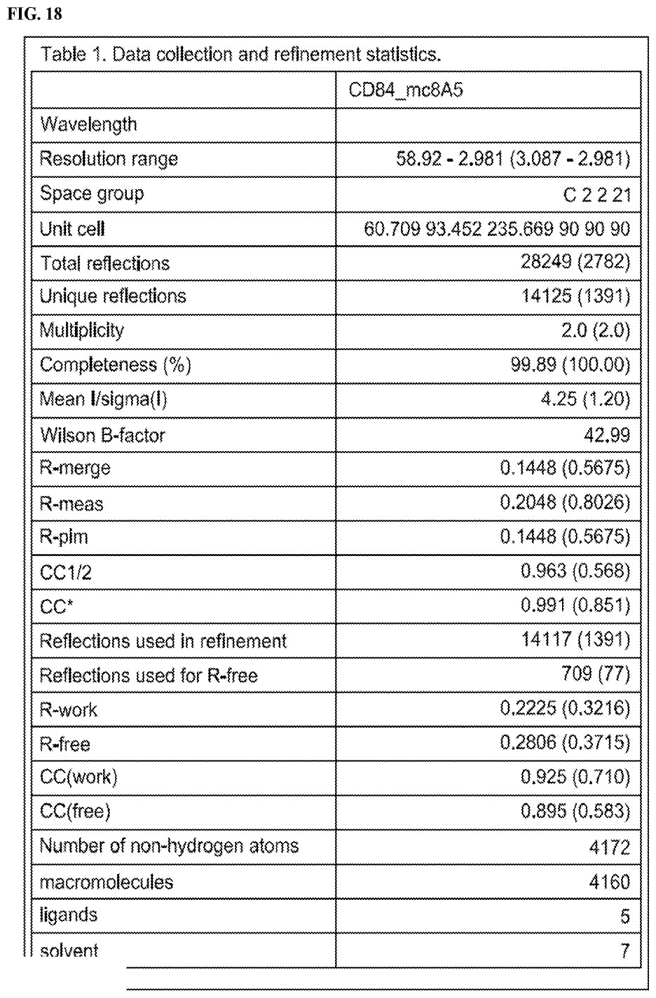

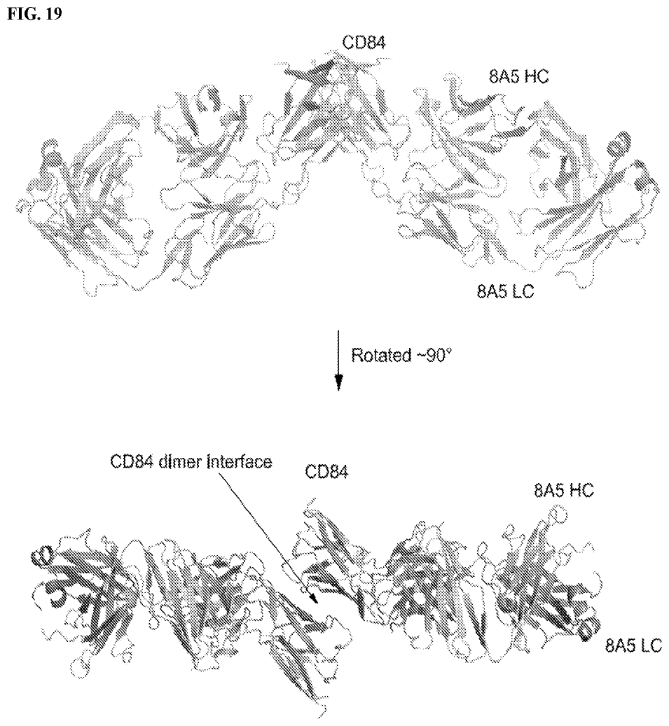

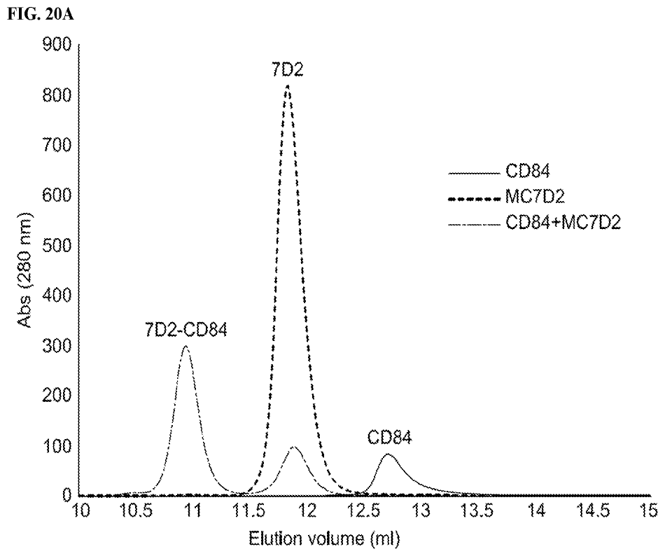

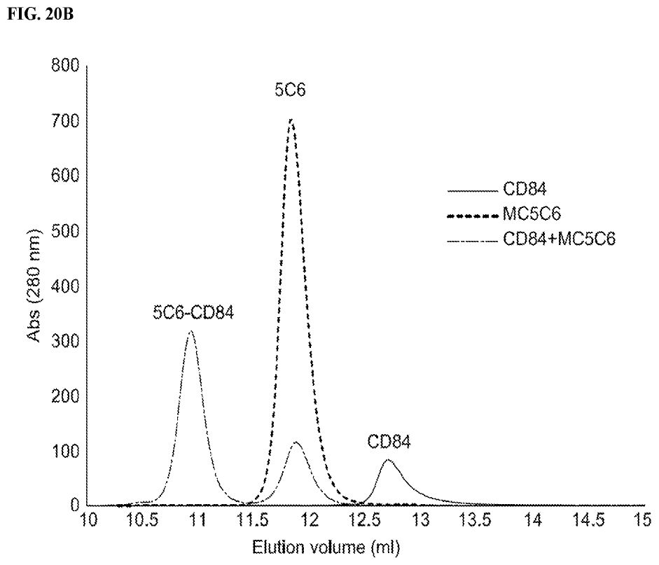

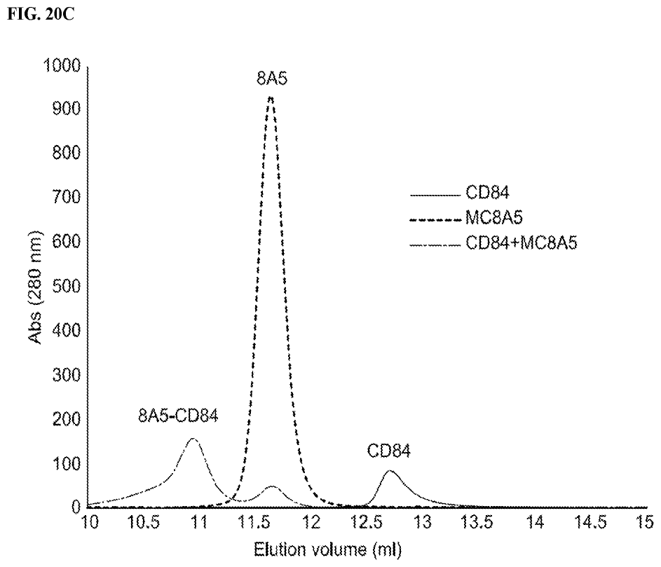

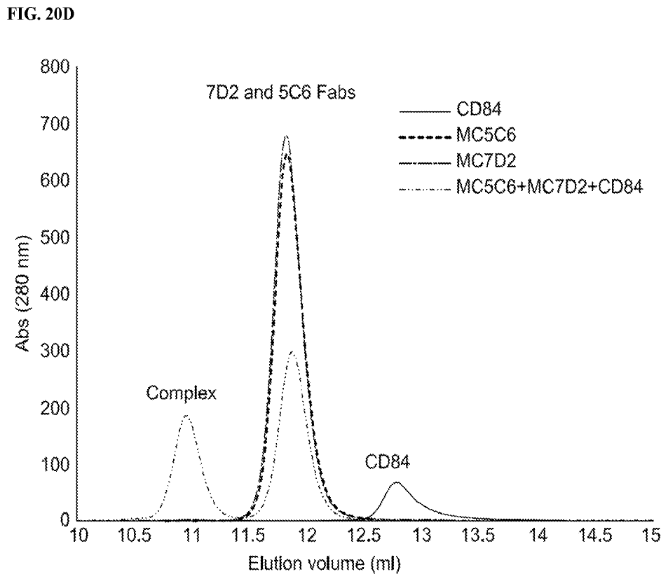

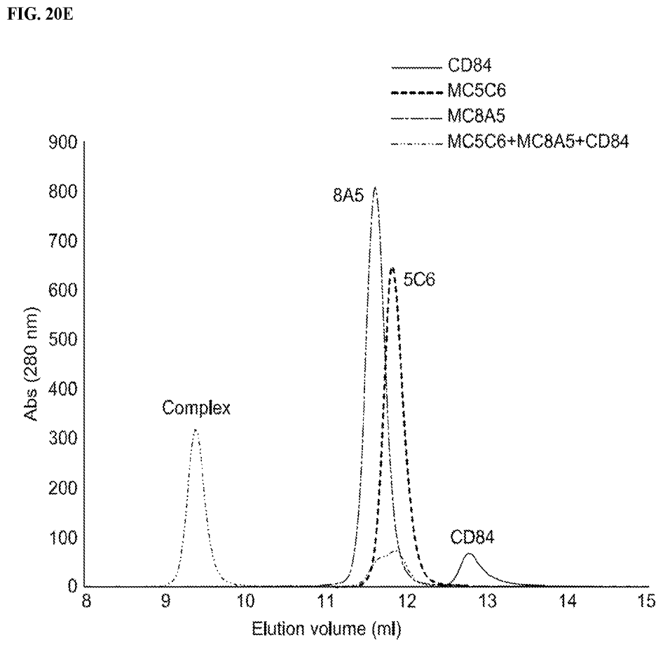

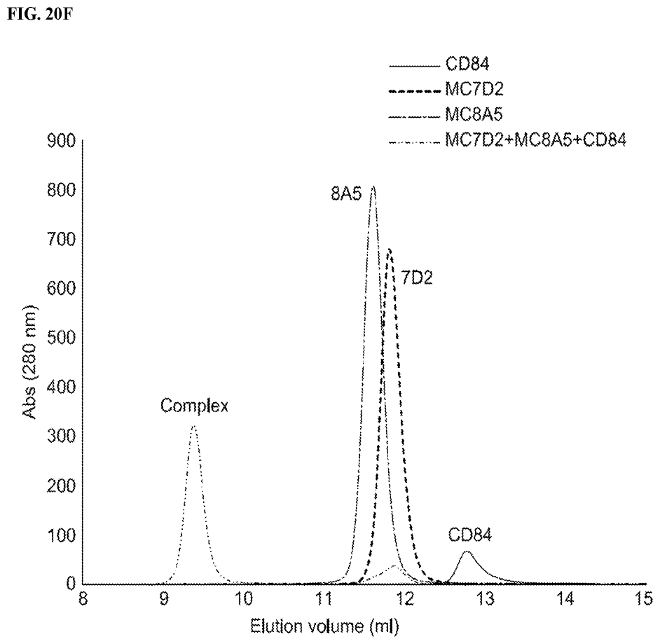

To efficiently target CD84 expressed AML cells in vivo, we generated three mouse anti-human chimeric monoclonal antibodies against CD84 (anti-CD84 antibody), including the monoclonal antibody 5C6, 7D2 and 8A5 among others. Antibody-Dependent Cellular Cytotoxicity (ADCC) assay shows that anti-CD84 7D2 antibody induced higher cytotoxicity in CD84 high-expressed cells THP1, but not in CD84 null/low expressed cells MMIS (FIG. 15A). We employed anti-CD84 7D2 antibody for further study. As expected, anti-CD84 7D2 antibody induced strongly ADCC in THP1 cells in presence of total peripheral blood mononuclear cells (PBMCs) or purified NK cells as effector cells (E), which exhibited dependent on ratio of effector vs target cells, as well as antibody dose (FIG. 15B). Notably, decreased tumor burden as well as extended survival were observed in luciferase expressed THP1-xenograft NSG mice following anti-CD84 7D2 antibody treatment (FIG. 16A). Meanwhile, we keep the same treatment strategy in U937-Luc+xenograft mouse model. Mice treated with anti-CD84 antibody showed a significant reduction of disease burden by bioluminescence compared with the mice treated with IgG, accompanied by a significantly prolonged survival (FIG. 16B). Consistently, in AML Patient-Derived Xenograft (PDX) model, we found reduced engraftment (indicated by human CD45/CD33) in bone marrow, spleen and peripheral blood, as well as reduced splenomegaly after anti-CD84 antibody treatment for three weeks (FIG. 17). Overall, our results suggested that CD84 is a survival receptor in AML and anti-CD84 antibody is a promising therapy for AML patient.

### Example 8: CD84 and 8A5 Interface

The PISA (Proteins, Interfaces, Structures and Assemblies) tool was used to calculate the buried surface area of CD84/8A5 structure. The interface area (in Å2, calculated as difference in total accessible surface areas of isolated and interfacing structures divided by two) of the human heavy chain (HC) and CD84 is 684 Å2. Within the interface are a number of hydrogen bonds including Ser99 (HC)-Gln24 (CD84), Thr53 (HC)-Gln24 (CD84), Ser33 (HC)-Glu25 (CD84); Thr30 (HC)-Gln24 (CD84) and and salt bridges His25 (HC) Glu25 (CD84).

The list of CD84 residues buried at the HC-CD84 interface is Val21, Asn22, ILe23, Gln24, Glu25, Pro26, Arg27, Gln28, Ser48, Glu49 Pro70, Tyr72, Ala95, Asp96, Pro97, Tyr98, Thr100, Lys102

The list of 8A5 residues of the HC is Thr30, Thr31, Tyr32, Ser33, His35, Trp47, Trp50, Asp52, Thr53, Ala54, Thr55, Glu57, Pro58, Thr59, Tyr60, Ala61, Lys65, Ser99, Tyr101, Trp102, Tyr103, Phe104

The interface between the human light chain (LC) and CD84 is 472 Å2 Within the interface are a number of hydrogen bonds including Asn 33 (LC)-Ser48 (CD84), Lys55 (LC)-Glu49 (CD84), Gly96 (LC)-Arg27 (CD84) and Asn35 (LC)-Ser48 (CD84) and salt bridges Lys55 (LC)-Glu49 (CD84) and Glu39 (LC)-Arg27 (CD84).

The list of CD84 residues buried at the LC-CD84 interface is Glu25, Arg27, Gln28, Val29, Lys30, Pro45, Gly46, Ssp47, Ser48, Glu49, Gln94 and Ala95

The list of 8A5 residues of the LC is His31, Ile32, Asn33, Gly34, Asn35, Tyr37, Glu39, Lys55, Phe94, Gly96, Ser97, His98, Vla99, Trp101

INFORMAL SEQUENCE LISTING

| SEQ ID NO: | Name of sequence | Sequence |
|---|---|---|
| 1. | 4B11 CDR H1 | GFTFSDF |
| 2. | 4B11 CDR H2 | RNKANDY |
| 3. | 4B11 CDR H3 | DAVWEPFAY |
| 4. | 4B11 CDR L1 | SANSSISSNSLH |
| 5. | 4B11 CDR L2 | RTSNLAS |
| 6. | 4B11 CDR L3 | QQGTSIPRT |
| 7. | 4B11 FR H1 | EVKLVESGGGLVQPGGSLRLSCATS |
| 8. | 4B11 FR H2 | YMDWVRQPPGKRLEWIATS |
| 9. | 4B11 FR H3 | TTEYSASVRGRFIVSRDTSQSILYLQMNALRAEDTAIYY |
| | | CAR |
| 10. | 4B11 FR H4 | WGQGTLVTVSA |
| 11. | 4B11 FR L1 | EIVLTQSPTTMAASPGEKITVTC |
| 12. | 4B11 FR L2 | WYQQKPGFSPKLLIY |
| 13. | 4B11 FR L3 | GVPARFSGSGSGTSYFLTIGTMEAEDVATYYC |
| 14. | 4B11 FR L4 | FGGGTKLEIK |
| 15. | 4B11 VH | EVKLVESGGGLVQPGGSLRLSCATSGFTFSDFYMDWVR |
| | | QPPGKRLEWIATSRNKANDYTTEYSASVRGRFIVSRDTS |
| | | QSILYLQMNALRAEDTAIYYCARDAVWEPFAYWGQGT |
| | | LVTVSA |
| 16. | 4B11 VL | EIVLTQSPTTMAASPGEKITVTCSANSSISSNSLHWYQQK |
| | | PGFSPKLLIYRTSNLASGVPARFSGSGSGTSYFLTIGTME |
| | | AEDVATYYCQQGTSIPRTFGGGTKLEIK |
| 17. | 4B11 HC | EVKLVESGGGLVQPGGSLRLSCATSGFTFSDFYMDWVR |
| | | QPPGKRLEWIATSRNKANDYTTEYSASVRGRFIVSRDTS |
| | | QSILYLQMNALRAEDTAIYYCARDAVWEPFAYWGQGT |
| | | LVTVSAASTKGPSVFPLAPSSKSTSGGTAALGCLVKDYF |
| | | PEPVTVSWNSGALTSGVHTFPAVLQSSGLYSLSSVVTVP |
| | | SSSLGTQTYICNVNHKPSNTKVDKKVEPKSC |
| 18. | 4B11 LC | EIVLTQSPTTMAASPGEKITVTCSANSSISSNSLHWYQQK |
| | | PGFSPKLLIYRTSNLASGVPARFSGSGSGTSYFLTIGTME |
| | | AEDVATYYCQQGTSIPRTFGGGTKLEIKRTVAAPSVFIFP |
| | | PSDEQLKSGTASVVCLLNNFYPREAKVQWKVDNALQS |
| | | GNSQESVTEQDSKDSTYSLSSTLTLSKADYEKHKVYAC |
| | | EVTHQGLSSPVTKSFNRGEC |
| 19. | 13D11 CDR H1 | GFSLNTSNM |
| 20. | 13D11 CDR H2 | WWDD |
| 21. | 13D11 CDR H3 | MRGDYTMDY |
| 22. | 13D11 CDR L1 | RASQSVSTSLYSYMH |
| 23. | 13D11 CDR L2 | YASDLES |
| 24. | 13D11 CDR L3 | QHSWEIPYT |
| 25. | 13D11 FR H1 | QVTLKESGPGLLQPSQTLSLTCSFS |
| 26. | 13D11 FR H2 | GVGWIRQPSGKDLEWLAHI |
| 27. | 13D11 FR H3 | VKRYNPALKSRLTISKDTSNNQVFLKIASVDTADTATYY |
| | | CAR |
| 28. | 13D11 FR H4 | WGQGTSVTVSS |
| 29. | 13D11 FR L1 | DIVLTQSPASLAVSLGQRATLSC |
| 30. | 13D11 FR L2 | WYQQKPGQPPKLLIK |
| 31. | 13D11 FR L3 | GVPARFSGSGSGTDFTLHIHPVEEEDTATYYC |
| 32. | 13D11 FR L4 | FGGGTKLEIK |
| 33. | 13D11 VH | QVTLKESGPGLLQPSQTLSLTCSFSGFSLNTSNMGVGWI |
| | | RQPSGKDLEWLAHIWWDDVKRYNPALKSRLTISKDTSN |
| | | NQVFLKIASVDTADTATYYCARMRGDYTMDYWGQGT |
| | | SVTVSS |
| 34. | 13D11 VL | DIVLTQSPASLAVSLGQRATLSCRASQSVSTSLYSYMHW |
| | | YQQKPGQPPKLLIKYASDLESGVPARFSGSGSGTDFTLHI |
| | | HPVEEEDTATYYCQHSWEIPYTFGGGTKLEIK |
| 35. | 13D11 HC | QVTLKESGPGLLQPSQTLSLTCSFSGFSLNTSNMGVGWI |
| | | RQPSGKDLEWLAHIWWDDVKRYNPALKSRLTISKDTSN |
| | | NQVFLKIASVDTADTATYYCARMRGDYTMDYWGQGT |
| | | SVTVSSASTKGPSVFPLAPSSKSTSGGTAALGCLVKDYF |
| | | PEPVTVSWNSGALTSGVHTFPAVLQSSGLYSLSSVVTVP |
| | | SSSLGTQTYICNVNHKPSNTKVDKKVEPKSC |
| 36. | 13D11 LC | DIVLTQSPASLAVSLGQRATLSCRASQSVSTSLYSYMHW |
| | | YQQKPGQPPKLLIKYASDLESGVPARFSGSGSGTDFTLHI |
| | | HPVEEEDTATYYCQHSWEIPYTFGGGTKLEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| 37. | 13D11 Hu HC | EIQLVESGGGLVKPGGSLRLSCAASGYTFTTYSMHWVR |
| | | QAPGKGLEWMGWIDTATGEPTLADDFKGRFTFSLDTSK |
| | | NTAYLQMNSLKIEDTAVYFCVKSAYWYFDVWGKGTTV |
| | | TVSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSC |
| 38. | 13D11 Hu LC | EVVMTQSPGTLSLSPGERATLSCRSSQSIVHINGNTYLE |
| | | WYQQKPGQAPRLLIYKVSNRFSGIPDRFSGSGSGTDFTL |
| | | TISRLEPEDFAVYYCFQGSHVPWTFGPGTKVDIKRTVAA |
| | | PSVFIFPPSDEQLKSGTASVVCLLNNFYPREAKVQWKVD |
| | | NALQSGNSQESVTEQDSKDSTYSLSSTLTLSKADYEKHK |
| | | VYACEVTHQGLSSPVTKSFNRGEC |
| 39. | 2H7 CDR H1 | GYSITSDY |
| 40. | 2H7 CDR H2 | SYSGT |
| 41. | 2H7 CDR H3 | LHYYGSRYKKLYAMDY |
| 42. | 2H7 CDR L1 | KASDNVGTYVS |
| 43. | 2H7 CDR L2 | GASNRYT |
| 44. | 2H7 CDR L3 | GQSYSYPT |
| 45. | 2H7 FR H1 | DVQLQESGPGLVKPSQSLSLTCTVT |
| 46. | 2H7 FR H2 | AWNWIRQFPGNKLEWMGYI |
| 47. | 2H7 FR H3 | TSYNPSLKSRISITRDTSKNQFFLQLNSVTTEDTATYYCA |
| | | S |
| 48. | 2H7 FR H4 | WGQGTLATVSS |
| 49. | 2H7 FR L1 | NIVMTQSPKSMSMSVGERVTLSC |
| 50. | 2H7 FR L2 | WYQQKPEQSPKLLIY |
| 51. | 2H7 FR L3 | GVPDRFTGSGSATDFTLTISSVQAEDLADYHC |
| 52. | 2H7 FR L4 | FGGGTKLEIK |
| 53. | 2H7 VH | DVQLQESGPGLVKPSQSLSLTCTVTGYSITSDYAWNWIR |
| | | QFPGNKLEWMGYISYSGTTSYNPSLKSRISITRDTSKNQF |
| | | FLQLNSVTTEDTATYYCASLHYYGSRYKKLYAMDYWG |
| | | QGTLATVSS |
| 54. | 2H7 VL | NIVMTQSPKSMSMSVGERVTLSCKASDNVGTYVSWYQ |
| | | QKPEQSPKLLIYGASNRYTGVPDRFTGSGSATDFTLTISS |
| | | VQAEDLADYHCGQSYSYPTFGGGTKLEIK |
| 55. | 2H7 HC | DVQLQESGPGLVKPSQSLSLTCTVTGYSITSDYAWNWIR |
| | | QFPGNKLEWMGYISYSGTTSYNPSLKSRISITRDTSKNQF |
| | | FLQLNSVTTEDTATYYCASLHYYGSRYKKLYAMDYWG |
| | | QGTLATVSSASTKGPSVFPLAPSSKSTSGGTAALGCLVK |
| | | DYFPEPVTVSWNSGALTSGVHTFPAVLQSSGLYSLSSVV |
| | | TVPSSSLGTQTYICNVNHKPSNTKVDKKVEPKSC |
| 56. | 2H7 LC | NIVMTQSPKSMSMSVGERVTLSCKASDNVGTYVSWYQ |
| | | QKPEQSPKLLIYGASNRYTGVPDRFTGSGSATDFTLTISS |
| | | VQAEDLADYHCGQSYSYPTFGGGTKLEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYA |
| | | CEVTHQGLSSPVTKSFNRGEC |
| 57. | 5C6 CDR H1 | GYTFTNY |
| 58. | 5C6 CDR H2 | FPGGGY |
| 59. | 5C6 CDR H3 | GNIAMDY |
| 60. | 5C6 CDR L1 | SASSSVSYVH |
| 61. | 5C6 CDR L2 | DTSKLAS |
| 62. | 5C6 CDR L3 | QQWNNSPRT |
| 63. | 5C6 FR H1 | QVHLQQSGAELVRPGTSVKISCKAS |
| 64. | 5C6 FR H2 | WLGWVKQRPGHGLEWIGDI |
| 65. | 5C6 FR H3 | INYHEKFKGKATLTADTSSSTAYMQLSSLTSEDSAVYFC |
| | | GY |
| 66. | 5C6 FR H4 | WGQGTSVTVSS |
| 67. | 5C6 FR L1 | QIILTQTPAIMSASPGEKVTMTC |
| 68. | 5C6 FR L2 | WYQQKSGTSPKRWIY |
| 69. | 5C6 FR L3 | GVPARFSGSGSGTSYSLTISTMEAEDAATYYC |
| 70. | 5C6 FR L4 | FGGGTKLEIK |
| 71. | 5C6 VH | QVHLQQSGAELVRPGTSVKISCKASGYTFTNYWLGWV |
| | | KQRPGHGLEWIGDIFPGGGYINYHEKFKGKATLTADTSS |
| | | STAYMQLSSLTSEDSAVYFCGYGNIAMDYWGQGTSVT |
| | | VSS |
| 72. | 5C6 VL | QIILTQTPAIMSASPGEKVTMTCSASSSVSYVHWYQQKS |
| | | GTSPKRWIYDTSKLASGVPARFSGSGSGTSYSLTISTME |
| | | AEDAATYYCQQWNNSPRTFGGGTKLEIK |
| 73. | 5C6 HC | QVHLQQSGAELVRPGTSVKISCKASGYTFTNYWLGWV |
| | | KQRPGHGLEWIGDIFPGGGYINYHEKFKGKATLTADTSS |
| | | STAYMQLSSLTSEDSAVYFCGYGNIAMDYWGQGTSVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSC |
| 74. | 5C6 LC | QIILTQTPAIMSASPGEKVTMTCSASSSVSYVHWYQQKS |
| | | GTSPKRWIYDTSKLASGVPARFSGSGSGTSYSLTISTME |
| | | AEDAATYYCQQWNNSPRTFGGGTKLEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYA |
| | | CEVTHQGLSSPVTKSFNRGEC |
| 75. | 7D2 CDR H1 | GYTFTNY |
| 76. | 7D2 CDR H2 | FPGGGY |
| 77. | 7D2 CDR H3 | GNVAMDY |
| 78. | 7D2 CDR L1 | SASSSVSYVH |
| 79. | 7D2 CDR L2 | DTSKLAS |
| 80. | 7D2 CDR L3 | QQWNNSPRT |
| 81. | 7D2 FR H1 | QVHLQQSGAELVRPGTSVKISCKAS |
| 82. | 7D2 FR H2 | WLGWVKQRPGHGLEWIGDI |
| 83. | 7D2 FR H3 | INYHEKFKGKATLTADTSSSTAYMQLSSLTSEDSAVYFC |
| | | AY |
| 84. | 7D2 FR H4 | WGQGTSVTVSS |
| 85. | 7D2 FR L1 | QIILTQTPAIMSASPGEKVTVTC |
| 86. | 7D2 FR L2 | WYQQKSGTSPKRWIY |
| 87. | 7D2 FR L3 | GVPARFSGSGSGTSYSLTISTMEAEDAATYYC |
| 88. | 7D2 FR L4 | FGGGTKLEIK |
| 89. | 7D2 VH | QVHLQQSGAELVRPGTSVKISCKASGYTFTNYWLGWV |
| | | KQRPGHGLEWIGDIFPGGGYINYHEKFKGKATLTADTSS |
| | | STAYMQLSSLTSEDSAVYFCAYGNVAMDYWGQGTSVT |
| | | VSS |
| 90. | 7D2 VL | QIILTQTPAIMSASPGEKVTVTCSASSSVSYVHWYQQKS |
| | | GTSPKRWIYDTSKLASGVPARFSGSGSGTSYSLTISTME |
| | | AEDAATYYCQQWNNSPRTFGGGTKLEIK |
| 91. | 7D2 HC | QVHLQQSGAELVRPGTSVKISCKASGYTFTNYWLGWV |
| | | KQRPGHGLEWIGDIFPGGGYINYHEKFKGKATLTADTSS |
| | | STAYMQLSSLTSEDSAVYFCAYGNVAMDYWGQGTSVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSC |
| 92. | 7D2 LC | QIILTQTPAIMSASPGEKVTVTCSASSSVSYVHWYQQKS |
| | | GTSPKRWIYDTSKLASGVPARFSGSGSGTSYSLTISTME |
| | | AEDAATYYCQQWNNSPRTFGGGTKLEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYA |
| | | CEVTHQGLSSPVTKSFNRGEC |
| 93. | 8A5 CDR H1 | GYTFTTY |
| 94. | 8A5 CDR H2 | DTATGE |
| 95. | 8A5 CDR H3 | SAYWYFDV |
| 96. | 8A5 CDR L1 | RSSQSIVHINGNTYLE |
| 97. | 8A5 CDR L2 | KVSNRFS |
| 98. | 8A5 CDR L3 | FQGSHVPWT |
| 99. | 8A5 FR H1 | QIQLVQSGPELKKPGETVKISCKAS |
| 100. | 8A5 FR H2 | SMHWVKQAPGKGLKWMGWI |
| 101. | 8A5 FR H3 | PTYADDFKGRFAFSLETSASTAYLQINNLNNDDTATYFC |
| | | VK |
| 102. | 8A5 FR H4 | WGAGTTVTVSS |
| 103. | 8A5 FR L1 | DVLMTQTPLSLPVSLGDQASISC |
| 104. | 8A5 FR L2 | WYLQKPGQSPKLLIY |
| 105. | 8A5 FR L3 | GVPDRFSGSGSGTDFTLKISRVEAEDLGVYYC |
| 106. | 8A5 FR L4 | FGGGTKLEIK |
| 107. | 8A5 VH | QIQLVQSGPELKKPGETVKISCKASGYTFTTYSMHWVK |
| | | QAPGKGLKWMGWIDTATGEPTYADDFKGRFAFSLETS |
| | | ASTAYLQINNLNNDDTATYFCVKSAYWYFDVWGAGTT |
| | | VTVSS |
| 108. | 8A5 VL | DVLMTQTPLSLPVSLGDQASISCRSSQSIVHINGNTYLE |
| | | WYLQKPGQSPKLLIYKVSNRFSGVPDRFSGSGSGTDFTL |
| | | KISRVEAEDLGVYYCFQGSHVPWTFGGGTKLEIK |
| 109. | 8A5 HC | QIQLVQSGPELKKPGETVKISCKASGYTFTTYSMHWVK |
| | | QAPGKGLKWMGWIDTATGEPTYADDFKGRFAFSLETS |
| | | ASTAYLQINNLNNDDTATYFCVKSAYWYFDVWGAGTT |
| | | VTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKKVEPKSC |
| 110. | 8A5 LC | DVLMTQTPLSLPVSLGDQASISCRSSQSIVHINGNTYLE |
| | | WYLQKPGQSPKLLIYKVSNRFSGVPDRFSGSGSGTDFTL |
| | | KISRVEAEDLGVYYCFQGSHVPWTFGGGTKLEIKRTVA |
| | | APSVFIFPPSDEQLKSGTASVVCLLNNFYPREAKVQWKV |
| | | DNALQSGNSQESVTEQDSKDSTYSLSSTLTLSKADYEKH |
| | | KVYACEVTHQGLSSPVTKSFNRGEC |
| 111. | CD84-IgG2A | KDSEIFTVNGILGESVTFPVNIQEPRQVKIIAWTSKTSVA |
| | | YVTPGDSETAPVVTVTHRNYYERIHALGPNYNLVISDLR |
| | | MEDAGDYKADINTQADPYTTTKRYNLQIYRRLGKPKIT |
| | | QSLMASVNSTCNVTLTCSVEKEEKNVTYNWSPLGEEGN |
| | | VLQIFQTPEDQELTYTCTAQNPVSNNSDSISARQLCADIA |
| | | MGFRTHHTGGGGSKPCPPCKCPAPNLLGGPSVFIFPPKIK |
| | | DVLMISLSPIVTCVVVDVSEDDPDVQISWFVNNVEVHT |
| | | AQTQTHREDYNSTLRVVSALPIQHQDWMSGKEFKCKV |
| | | NNKDLPAPIERTISKPKGSVRAPQVYVLPPPEEEMTKKQ |
| | | VTLTCMVTDFMPEDIYVEWTNNGKTELNYKNTEPVLDS |
| | | DGSYFMYSKLRVEKKNWVERNSYSCSVVHEGLHNHHT |
| | | TKSFSRTPGK |

### P EMBODIMENTS

P Embodiment 1. An anti-CD84 antibody comprising a light chain variable domain and a heavy chain variable domain, wherein said heavy chain variable domain comprises a CDR H1 as set forth in SEQ ID NO:75, a CDR H2 as set forth in SEQ ID NO:76, and a CDR H3 as set forth in SEQ ID NO:77; and wherein said light chain variable domain comprises a CDR L1 as set forth in SEQ ID NO:78, a CDR L2 as set forth in SEQ ID NO:79 and a CDR L3 as set forth in SEQ ID NO: 80.

P Embodiment 2. The anti-CD84 antibody of P embodiment 1, wherein said anti-CD84 antibody is a chimeric antibody.

P Embodiment 3. The anti-CD84 antibody of P embodiment 1 or 2, wherein said heavy chain variable domain comprises a FR H1 as set forth in SEQ ID NO:81, a FR H2 as set forth in SEQ ID NO:82, a FR H3 as set forth in SEQ ID NO:83 and a FR H4 as set forth in SEQ ID NO: 84.

P Embodiment 4. The anti-CD84 antibody of any one of P embodiments 1-3, wherein said light chain variable domain comprises a FR L1 as set forth in SEQ ID NO:85, a FR L2 as set forth in SEQ ID NO:86, a FR L3 as set forth in SEQ ID NO:87 and a FR L4 as set forth in SEQ ID NO: 88.

P Embodiment 5. The anti-CD84 antibody of any one of P embodiments 1˜4 wherein said heavy chain variable domain comprises the sequence of SEQ ID NO:89.

P Embodiment 6. The anti-CD84 antibody of any one of P embodiments 1-5, wherein said light chain variable domain comprises the sequence of SEQ ID NO:90.

P Embodiment 7. The anti-CD84 antibody of any one of P embodiments 1-6, wherein said anti-CD84 antibody comprises a heavy chain comprising the sequence of SEQ ID NO:91.

P Embodiment 8. The anti-CD84 antibody of any one of P embodiments 1-7, wherein said anti-CD84 antibody comprises a light chain comprising the sequence of SEQ ID NO:92.

P Embodiment 9. An anti-CD84 antibody comprising a light chain variable domain and a heavy chain variable domain, wherein said heavy chain variable domain comprises a CDR H1 as set forth in SEQ ID NO:19, a CDR H2 as set forth in SEQ ID NO:20, and a CDR H3 as set forth in SEQ ID NO:21; and wherein said light chain variable domain comprises a CDR L1 as set forth in SEQ ID NO:22, a CDR L2 as set forth in SEQ ID NO:23 and a CDR L3 as set forth in SEQ ID NO: 24.

P Embodiment 10. The anti-CD84 antibody of P embodiments 9, wherein said anti-CD84 antibody is a humanized antibody.

P Embodiment 11. The anti-CD84 antibody of any one of P embodiments 9-10, wherein said antibody comprises a light chain comprising the sequence of SEQ ID NO:37.

P Embodiment 12. The anti-CD84 antibody of any one of P embodiments 9-11, wherein said antibody comprises a heavy chain comprising the sequence of SEQ ID NO:38

P Embodiment 13. The anti-CD84 antibody of P embodiment 9, wherein said anti-CD84 antibody is a chimeric antibody.

P Embodiment 14. The anti-CD84 antibody of P embodiment 9 or 13, wherein said heavy chain variable domain comprises a FR H1 as set forth in SEQ ID NO:25, a FR H2 as set forth in SEQ ID NO:26, a FR H3 as set forth in SEQ ID NO:27 and a FR H4 as set forth in SEQ ID NO: 28.

P Embodiment 15. The anti-CD84 antibody of any one of P embodiments 9 or 13-14, wherein said light chain variable domain comprises a FR L1 as set forth in SEQ ID NO:29, a FR L2 as set forth in SEQ ID NO:30, a FR L3 as set forth in SEQ ID NO:31 and a FR L4 as set forth in SEQ ID NO:32.

P Embodiment 16. The anti-CD84 antibody of any one of P embodiments 9 or 13-15, wherein said heavy chain variable domain comprises the sequence of SEQ ID NO:33.

P Embodiment 17. The anti-CD84 antibody of any one of P embodiments 9 or 13-16, wherein said light chain variable domain comprises the sequence of SEQ ID NO:34.

P Embodiment 18. The anti-CD84 antibody of any one of P embodiments 9 or 13-17, wherein said anti-CD84 antibody comprises a heavy chain comprising the sequence of SEQ ID NO: 35.

P Embodiment 19. The anti-CD84 antibody of any one of P embodiments 9 or 13-18, wherein said anti-CD84 antibody comprises a light chain comprising the sequence of SEQ ID NO: 36.

P Embodiment 20. An anti-CD84 antibody comprising a light chain variable domain and a heavy chain variable domain, wherein said heavy chain variable domain comprises a CDR H1 as set forth in SEQ ID NO:39, a CDR H2 as set forth in SEQ ID NO:40, and a CDR H3 as set forth in SEQ ID NO:41; and wherein said light chain variable domain comprises a CDR L1 as set forth in SEQ ID NO:42, a CDR L2 as set forth in SEQ ID NO:43 and a CDR L3 as set forth in SEQ ID NO: 44.

P Embodiment 21. The anti-CD84 antibody of P embodiment 20, wherein said anti-CD84 antibody is a chimeric antibody.

P Embodiment 22. The anti-CD84 antibody of P embodiment 20 or 21, wherein said heavy chain variable domain comprises a FR H1 as set forth in SEQ ID NO:45, a FR H2 as set forth in SEQ ID NO:46, a FR H3 as set forth in SEQ ID NO:47 and a FR H4 as set forth in SEQ ID NO: 48.

P Embodiment 23. The anti-CD84 antibody of any one of P embodiments 20-22, wherein said light chain variable domain comprises a FR L1 as set forth in SEQ ID NO:49, a FR L2 as set forth in SEQ ID NO:50, a FR L3 as set forth in SEQ ID NO:51 and a FR L4 as set forth in SEQ ID NO: 52.

P Embodiment 24. The anti-CD84 antibody of any one of P embodiments 20-23 wherein said heavy chain variable domain comprises the sequence of SEQ ID NO:53.

P Embodiment 25. The anti-CD84 antibody of any one of P embodiments 20-24, wherein said light chain variable domain comprises the sequence of SEQ ID NO:54.

P Embodiment 26. The anti-CD84 antibody of any one of P embodiments 20-25, wherein said anti-CD84 antibody comprises a heavy chain comprising the sequence of SEQ ID NO:55.

P Embodiment 27. The anti-CD84 antibody of any one of P embodiments 20-26, wherein said anti-CD84 antibody comprises a light chain comprising the sequence of SEQ ID NO:56.

P Embodiment 28. An anti-CD84 antibody comprising a light chain variable domain and a heavy chain variable domain, wherein said heavy chain variable domain comprises a CDR H1 as set forth in SEQ ID NO:57, a CDR H2 as set forth in SEQ ID NO:58, and a CDR H3 as set forth in SEQ ID NO:59; and wherein said light chain variable domain comprises a CDR L1 as set forth in SEQ ID NO:60, a CDR L2 as set forth in SEQ ID NO:61 and a CDR L3 as set forth in SEQ ID NO: 62.

P Embodiment 29. The anti-CD84 antibody of P embodiment 28, wherein said anti-CD84 antibody is a chimeric antibody.

P Embodiment 30. The anti-CD84 antibody of P embodiment 28 or 29, wherein said heavy chain variable domain comprises a FR H1 as set forth in SEQ ID NO:63, a FR H2 as set forth in SEQ ID NO:64, a FR H3 as set forth in SEQ ID NO:65 and a FR H4 as set forth in SEQ ID NO: 66.

P Embodiment 31. The anti-CD84 antibody of any one of P embodiments 28-30, wherein said light chain variable domain comprises a FR L1 as set forth in SEQ ID NO:67, a FR L2 as set forth in SEQ ID NO:68, a FR L3 as set forth in SEQ ID NO:69 and a FR L4 as set forth in SEQ ID NO: 70.

P Embodiment 32. The anti-CD84 antibody of any one of P embodiments 28-31 wherein said heavy chain variable domain comprises the sequence of SEQ ID NO:71.

P Embodiment 33. The anti-CD84 antibody of any one of P embodiments 28-32, wherein said light chain variable domain comprises the sequence of SEQ ID NO:72.

P Embodiment 34. The anti-CD84 antibody of any one of P embodiments 28-33, wherein said anti-CD84 antibody comprises a heavy chain comprising the sequence of SEQ ID NO:73.

P Embodiment 35. The anti-CD84 antibody of any one of P embodiments 28-34, wherein said anti-CD84 antibody comprises a light chain comprising the sequence of SEQ ID NO:74.

P Embodiment 36. An anti-CD84 antibody comprising a light chain variable domain and a heavy chain variable domain, wherein said heavy chain variable domain comprises a CDR H1 as set forth in SEQ ID NO:93, a CDR H2 as set forth in SEQ ID NO:94, and a CDR H3 as set forth in SEQ ID NO:95; and wherein said light chain variable domain comprises a CDR L1 as set forth in SEQ ID NO:96, a CDR L2 as set forth in SEQ ID NO:97 and a CDR L3 as set forth in SEQ ID NO: 98.

P Embodiment 37. The anti-CD84 antibody of P embodiment 36, wherein said anti-CD84 antibody is a chimeric antibody.

P Embodiment 38. The anti-CD84 antibody of P embodiment 36 or 37, wherein said heavy chain variable domain comprises a FR H1 as set forth in SEQ ID NO:99, a FR H2 as set forth in SEQ ID NO: 100, a FR H3 as set forth in SEQ ID NO: 101 and a FR H4 as set forth in SEQ ID NO: 102.

P Embodiment 39. The anti-CD84 antibody of any one of P embodiments 36-38, wherein said light chain variable domain comprises a FR L1 as set forth in SEQ ID NO: 103, a FR L2 as set forth in SEQ ID NO: 104, a FR L3 as set forth in SEQ ID NO: 105 and a FR L4 as set forth in SEQ ID NO: 106.

P Embodiment 40. The anti-CD84 antibody of any one of P embodiments 36-39 wherein said heavy chain variable domain comprises the sequence of SEQ ID NO:107.

P Embodiment 41. The anti-CD84 antibody of any one of P embodiments 36-40, wherein said light chain variable domain comprises the sequence of SEQ ID NO: 108.

P Embodiment 42. The anti-CD84 antibody of any one of P embodiments 36-41, wherein said anti-CD84 antibody comprises a heavy chain comprising the sequence of SEQ ID NO: 109.

P Embodiment 43. The anti-CD84 antibody of any one of P embodiments 36-42, wherein said anti-CD84 antibody comprises a light chain comprising the sequence of SEQ ID NO:110.

P Embodiment 44. The anti-CD84 antibody of any one of P embodiments 1-43, wherein said anti-CD84 antibody is a Fab′ fragment.

P Embodiment 45. The anti-CD84 antibody of any one of P embodiments 1-44, wherein said anti-CD84 antibody is an IgG.

P Embodiment 46. The anti-CD84 antibody of any one of P embodiments 1, 3-6, 9, 14-17, 20, 22-25, 28, 30-33, or 36, or 38-41, wherein said light chain variable domain and said heavy chain variable domain form part of a scFv.

P Embodiment 47. The anti-CD84 antibody of any one of P embodiments 1-46, wherein said anti-CD84 antibody is capable of binding a CD84 protein.

P Embodiment 48. The anti-CD84 antibody of any one of P embodiments 1-47, wherein said anti-CD84 antibody is bound to a CD84 protein.

P Embodiment 49. The anti-CD84 antibody of P embodiment 47, wherein said CD84 protein is a human CD84 protein.

P Embodiment 50. The anti-CD84 antibody of P embodiment 48 or 49, wherein said CD84 protein forms part of a cell.

P Embodiment 51. The anti-CD84 antibody of any one of P embodiments 47-50, wherein said CD84 protein is expressed on the surface of a cell.

P Embodiment 52. The anti-CD84 antibody of P embodiment 50 or 51, wherein said cell is a cancer cell.

P Embodiment 53. The anti-CD84 antibody of P embodiment 52, wherein said cancer cell is a leukemia cancer cell, a myeloid cancer cell, or a lymphoma cancer cell.

P Embodiment 54. An anti-CD84 antibody, wherein said anti-CD84 antibody binds the same epitope as an antibody comprising: a heavy chain variable domain comprising a CDR H1 as set forth in SEQ ID NO:19, a CDR H2 as set forth in SEQ ID NO:20, and a CDR H3 as set forth in SEQ ID NO:21, and a light chain variable domain comprising a CDR L1 as set forth in SEQ ID NO: 22, a CDR L2 as set forth in SEQ ID NO:23 and a CDR L3 as set forth in SEQ ID NO:24.

P Embodiment 55. An anti-CD84 antibody, wherein said anti-CD84 antibody binds the same epitope as an antibody comprising: a heavy chain variable domain comprising a CDR H1 as set forth in SEQ ID NO:39, a CDR H2 as set forth in SEQ ID NO:40, and a CDR H3 as set forth in SEQ ID NO:41, and a light chain variable domain comprising a CDR L1 as set forth in SEQ ID NO: 42, a CDR L2 as set forth in SEQ ID NO:43 and a CDR L3 as set forth in SEQ ID NO:44.

P Embodiment 56. An anti-CD84 antibody, wherein said anti-CD84 antibody binds the same epitope as an antibody comprising: a heavy chain variable domain comprising a CDR H1 as set forth in SEQ ID NO:57, a CDR H2 as set forth in SEQ ID NO:58, and a CDR H3 as set forth in SEQ ID NO:59, and a light chain variable domain comprising a CDR L1 as set forth in SEQ ID NO: 60, a CDR L2 as set forth in SEQ ID NO:61 and a CDR L3 as set forth in SEQ ID NO:62.

P Embodiment 57. An anti-CD84 antibody, wherein said anti-CD84 antibody binds the same epitope as an antibody comprising: a heavy chain variable domain comprising a CDR H1 as set forth in SEQ ID NO:75, a CDR H2 as set forth in SEQ ID NO:76, and a CDR H3 as set forth in SEQ ID NO:77, and a light chain variable domain comprising a CDR L1 as set forth in SEQ ID NO: 78, a CDR L2 as set forth in SEQ ID NO:79 and a CDR L3 as set forth in SEQ ID NO:80.

P Embodiment 58. An anti-CD84 antibody, wherein said anti-CD84 antibody binds the same epitope as an antibody comprising: a heavy chain variable domain comprising a CDR H1 as set forth in SEQ ID NO:93, a CDR H2 as set forth in SEQ ID NO:94, and a CDR H3 as set forth in SEQ ID NO:95, and a light chain variable domain comprising a CDR L1 as set forth in SEQ ID NO: 96, a CDR L2 as set forth in SEQ ID NO:97 and a CDR L3 as set forth in SEQ ID NO:98.

P Embodiment 59. The anti-CD84 antibody of any one of P embodiments 1-58, wherein said anti-CD84 antibody is attached to a therapeutic or a diagnostic moiety.

P Embodiment 60. The anti-CD84 antibody of P embodiment 59, wherein said therapeutic moiety is an anti-cancer moiety.

P Embodiment 61. An isolated nucleic acid encoding an anti-CD84 antibody of any one of P embodiments 1-60.

62. A cell comprising an anti-CD84 antibody of any one of claims 1-60, or a nucleic acid of P embodiment 61.

P Embodiment 63. A pharmaceutical composition comprising a therapeutically effective amount of an antibody of any of P embodiments 1-60 and a pharmaceutically acceptable excipient.

P Embodiment 64. A method of forming an antibody capable of binding to CD84, said method comprises immunizing a mammal with a peptide comprising the sequence of SEQ ID NO: 111.

P Embodiment 65. A method of treating cancer in a subject in need thereof, said method comprising administering to a subject a therapeutically effective amount of an anti-CD84 antibody of any one of P embodiments 1-60 or a pharmaceutical composition of P embodiment 63, thereby treating cancer in said subject.

P Embodiment 66. The method of treating cancer of P embodiment 65, wherein said cancer is lymphoma, myeloma, or leukemia.

P Embodiment 67. The method of treating cancer of P embodiment 65 or 66, wherein said cancer is acute myeloid leukemia (AML).

What is claimed is:

1. An anti-CD84 antibody comprising a light chain variable domain and a heavy chain variable domain, wherein said heavy chain variable domain comprises a CDR H1 as set forth in SEQ ID NO:75, a CDR H2 as set forth in SEQ ID NO:76, and a CDR H3 as set forth in SEQ ID NO:77; and
wherein said light chain variable domain comprises a CDR L1 as set forth in SEQ ID NO: 78, a CDR L2 as set forth in SEQ ID NO:79 and a CDR L3 as set forth in SEQ ID NO:80.

2. The anti-CD84 antibody of claim 1, wherein said anti-CD84 antibody is a chimeric antibody.

3. The anti-CD84 antibody of claim 1, wherein said heavy chain variable domain comprises a FR H1 as set forth in SEQ ID NO:81, a FR H2 as set forth in SEQ ID NO:82, a FR H3 as set forth in SEQ ID NO:83 and a FR H4 as set forth in SEQ ID NO:84.

4. The anti-CD84 antibody of claim 1, wherein said light chain variable domain comprises a FR L1 as set forth in SEQ ID NO:85, a FR L2 as set forth in SEQ ID NO:86, a FR L3 as set forth in SEQ ID NO:87 and a FR L4 as set forth in SEQ ID NO:88.

5. The anti-CD84 antibody of claim 1, wherein said heavy chain variable domain comprises the sequence of SEQ ID NO:89.

6. The anti-CD84 antibody of claim 1, wherein said light chain variable domain comprises the sequence of SEQ ID NO:90.

7. The anti-CD84 antibody of claim 1, wherein said anti-CD84 antibody comprises a heavy chain comprising the sequence of SEQ ID NO:91.

8. The anti-CD84 antibody of claim 1, wherein said anti-CD84 antibody comprises a light chain comprising the sequence of SEQ ID NO:92.

9. An anti-CD84 antibody comprising a light chain variable domain and a heavy chain variable domain, wherein said heavy chain variable domain comprises a CDR H1 as set forth in SEQ ID NO:19, a CDR H2 as set forth in SEQ ID NO:20, and a CDR H3 as set forth in SEQ ID NO:21; and
wherein said light chain variable domain comprises a CDR L1 as set forth in SEQ ID NO: 22, a CDR L2 as set forth in SEQ ID NO:23 and a CDR L3 as set forth in SEQ ID NO:24.

10. The anti-CD84 antibody of claim 9, wherein said anti-CD84 antibody is a humanized antibody.

11. The anti-CD84 antibody of claim 9, wherein said antibody comprises a light chain comprising the sequence of SEQ ID NO:37.

12. The anti-CD84 antibody of claim 9, wherein said antibody comprises a heavy chain comprising the sequence of SEQ ID NO:38.

13. The anti-CD84 antibody of claim 9, wherein said anti-CD84 antibody is a chimeric antibody.

14. The anti-CD84 antibody of claim 9, wherein said heavy chain variable domain comprises a FR H1 as set forth in SEQ ID NO:25, a FR H2 as set forth in SEQ ID NO:26, a FR H3 as set forth in SEQ ID NO:27 and a FR H4 as set forth in SEQ ID NO:28.

15. The anti-CD84 antibody of claim 9, wherein said light chain variable domain comprises a FR L1 as set forth in SEQ ID NO:29, a FR L2 as set forth in SEQ ID NO:30, a FR L3 as set forth in SEQ ID NO:31 and a FR L4 as set forth in SEQ ID NO:32.

16. The anti-CD84 antibody of claim 9, wherein said heavy chain variable domain comprises the sequence of SEQ ID NO:33.

17. The anti-CD84 antibody of claim 9, wherein said light chain variable domain comprises the sequence of SEQ ID NO:34.

18. The anti-CD84 antibody of claim 9, wherein said anti-CD84 antibody comprises a heavy chain comprising the sequence of SEQ ID NO:35.

19. The anti-CD84 antibody of claim 9, wherein said anti-CD84 antibody comprises a light chain comprising the sequence of SEQ ID NO:36.

20. An anti-CD84 antibody comprising a light chain variable domain and a heavy chain variable domain, wherein said heavy chain variable domain comprises a CDR H1 as set forth in SEQ ID NO:39, a CDR H2 as set forth in SEQ ID NO:40, and a CDR H3 as set forth in SEQ ID NO:41; and
wherein said light chain variable domain comprises a CDR L1 as set forth in SEQ ID NO: 42, a CDR L2 as set forth in SEQ ID NO:43 and a CDR L3 as set forth in SEQ ID NO:44.

21. The anti-CD84 antibody of claim 20, wherein said anti-CD84 antibody is a chimeric antibody.

22. The anti-CD84 antibody of claim 20, wherein said heavy chain variable domain comprises a FR H1 as set forth in SEQ ID NO:45, a FR H2 as set forth in SEQ ID NO:46, a FR H3 as set forth in SEQ ID NO:47 and a FR H4 as set forth in SEQ ID NO:48.

23. The anti-CD84 antibody of claim 20, wherein said light chain variable domain comprises a FR L1 as set forth in SEQ ID NO:49, a FR L2 as set forth in SEQ ID NO:50, a FR L3 as set forth in SEQ ID NO:51 and a FR L4 as set forth in SEQ ID NO:52.

24. The anti-CD84 antibody of claim 20, wherein said heavy chain variable domain comprises the sequence of SEQ ID NO:53.

25. The anti-CD84 antibody of claim 20, wherein said light chain variable domain comprises the sequence of SEQ ID NO:54.

26. The anti-CD84 antibody of claim 20, wherein said anti-CD84 antibody comprises a heavy chain comprising the sequence of SEQ ID NO:55.

27. The anti-CD84 antibody of claim 20, wherein said anti-CD84 antibody comprises a light chain comprising the sequence of SEQ ID NO:56.

28. An anti-CD84 antibody comprising a light chain variable domain and a heavy chain variable domain, wherein said heavy chain variable domain comprises a CDR H1 as set forth in SEQ ID NO:57, a CDR H2 as set forth in SEQ ID NO:58, and a CDR H3 as set forth in SEQ ID NO:59; and
wherein said light chain variable domain comprises a CDR L1 as set forth in SEQ ID NO: 60, a CDR L2 as set forth in SEQ ID NO:61 and a CDR L3 as set forth in SEQ ID NO:62.

29. The anti-CD84 antibody of claim 28, wherein said anti-CD84 antibody is a chimeric antibody.

30. The anti-CD84 antibody of claim 28, wherein said heavy chain variable domain comprises a FR H1 as set forth in SEQ ID NO:63, a FR H2 as set forth in SEQ ID NO:64, a FR H3 as set forth in SEQ ID NO:65 and a FR H4 as set forth in SEQ ID NO:66.

31. The anti-CD84 antibody of claim 28, wherein said light chain variable domain comprises a FR L1 as set forth in SEQ ID NO:67, a FR L2 as set forth in SEQ ID NO:68, a FR L3 as set forth in SEQ ID NO:69 and a FR L4 as set forth in SEQ ID NO:70.

32. The anti-CD84 antibody of claim 28, wherein said heavy chain variable domain comprises the sequence of SEQ ID NO:71.

33. The anti-CD84 antibody of claim 28, wherein said light chain variable domain comprises the sequence of SEQ ID NO:72.

34. The anti-CD84 antibody of claim 28, wherein said anti-CD84 antibody comprises a heavy chain comprising the sequence of SEQ ID NO:73.

35. The anti-CD84 antibody of claim 28, wherein said anti-CD84 antibody comprises a light chain comprising the sequence of SEQ ID NO:74.

36. An anti-CD84 antibody comprising a light chain variable domain and a heavy chain variable domain, wherein said heavy chain variable domain comprises a CDR H1 as set forth in SEQ ID NO:93, a CDR H2 as set forth in SEQ ID NO:94, and a CDR H3 as set forth in SEQ ID NO:95; and
wherein said light chain variable domain comprises a CDR L1 as set forth in SEQ ID NO: 96, a CDR L2 as set forth in SEQ ID NO:97 and a CDR L3 as set forth in SEQ ID NO:98.

37. The anti-CD84 antibody of claim 36, wherein said anti-CD84 antibody is a chimeric antibody.

38. The anti-CD84 antibody of claim 36, wherein said heavy chain variable domain comprises a FR H1 as set forth in SEQ ID NO:99, a FR H2 as set forth in SEQ ID NO: 100, a FR H3 as set forth in SEQ ID NO: 101 and a FR H4 as set forth in SEQ ID NO: 102.

39. The anti-CD84 antibody of claim 36, wherein said light chain variable domain comprises a FR L1 as set forth in SEQ ID NO: 103, a FR L2 as set forth in SEQ ID NO: 104, a FR L3 as set forth in SEQ ID NO: 105 and a FR L4 as set forth in SEQ ID NO: 106.

40. The anti-CD84 antibody of claim 36, wherein said heavy chain variable domain comprises the sequence of SEQ ID NO:107.

41. The anti-CD84 antibody of claim 36, wherein said light chain variable domain comprises the sequence of SEQ ID NO:108.

42. The anti-CD84 antibody of claim 36, wherein said anti-CD84 antibody comprises a heavy chain comprising the sequence of SEQ ID NO:109.

43. The anti-CD84 antibody of claim 36, wherein said anti-CD84 antibody comprises a light chain comprising the sequence of SEQ ID NO:110.

44. The anti-CD84 antibody of any one of claim 1, 9, 20, 28 or 36, wherein said anti-CD84 antibody is a Fab′ fragment.

45. The anti-CD84 antibody of any one of claim 1, 9, 20, 28 or 36, wherein said anti-CD84 antibody is an IgG.

46. The anti-CD84 antibody of any one of claim 1, 9, 20, 28 or 36, wherein said light chain variable domain and said heavy chain variable domain form part of a scFv.

47. The anti-CD84 antibody of any one of claim 1, 9, 20, 28 or 36, wherein said anti-CD84 antibody is capable of binding a CD84 protein.

48. The anti-CD84 antibody of any one of claim 1, 9, 20, 28 or 36, wherein said anti-CD84 antibody is bound to a CD84 protein.

49. The anti-CD84 antibody of claim 47, wherein said CD84 protein is a human CD84 protein.

50. The anti-CD84 antibody of claim 48, wherein said CD84 protein forms part of a cell.

51. The anti-CD84 antibody of claim 47, wherein said CD84 protein is expressed on the surface of a cell.

52. The anti-CD84 antibody of claim 50, wherein said cell is a cancer cell.

53. The anti-CD84 antibody of claim 52, wherein said cancer cell is a leukemia cancer cell, a myeloid cancer cell, or a lymphoma cancer cell.

54. An anti-CD84 antibody, wherein said anti-CD84 antibody binds the same epitope as an antibody comprising: a heavy chain variable domain comprising a CDR H1 as set forth in SEQ ID NO:19, a CDR H2 as set forth in SEQ ID NO:20, and a CDR H3 as set forth in SEQ ID NO:21, and a light chain variable domain comprising a CDR L1 as set forth in SEQ ID NO: 22, a CDR L2 as set forth in SEQ ID NO:23 and a CDR L3 as set forth in SEQ ID NO:24.

55. An anti-CD84 antibody, wherein said anti-CD84 antibody binds the same epitope as an antibody comprising: a heavy chain variable domain comprising a CDR H1 as set forth in SEQ ID NO:39, a CDR H2 as set forth in SEQ ID NO:40, and a CDR H3 as set forth in SEQ ID NO:41, and a light chain variable domain comprising a CDR L1 as set forth in SEQ ID NO: 42, a CDR L2 as set forth in SEQ ID NO:43 and a CDR L3 as set forth in SEQ ID NO:44.

56. An anti-CD84 antibody, wherein said anti-CD84 antibody binds the same epitope as an antibody comprising: a heavy chain variable domain comprising a CDR H1 as set forth in SEQ ID NO:57, a CDR H2 as set forth in SEQ ID NO:58, and a CDR H3 as set forth in SEQ ID NO:59, and a light chain variable domain comprising a CDR L1 as set forth in SEQ ID NO: 60, a CDR L2 as set forth in SEQ ID NO:61 and a CDR L3 as set forth in SEQ ID NO:62.

57. An anti-CD84 antibody, wherein said anti-CD84 antibody binds the same epitope as an antibody comprising: a heavy chain variable domain comprising a CDR H1 as set forth in SEQ ID NO:75, a CDR H2 as set forth in SEQ ID NO:76, and a CDR H3 as set forth in SEQ ID NO:77, and a light chain variable domain comprising a CDR L1 as set forth in SEQ ID NO: 78, a CDR L2 as set forth in SEQ ID NO:79 and a CDR L3 as set forth in SEQ ID NO:80.

58. An anti-CD84 antibody, wherein said anti-CD84 antibody binds the same epitope as an antibody comprising: a heavy chain variable domain comprising a CDR H1 as set forth in SEQ ID NO:93, a CDR H2 as set forth in SEQ ID NO:94, and a CDR H3 as set forth in SEQ ID NO:95, and a light chain variable domain comprising a CDR L1 as set forth in SEQ ID NO: 96, a CDR L2 as set forth in SEQ ID NO:97 and a CDR L3 as set forth in SEQ ID NO:98.

59. The anti-CD84 antibody of any one of claim 1, 9, 20, 28, 36, 54, 55, 56, 57 or 58, wherein said anti-CD84 antibody is attached to a therapeutic or a diagnostic moiety.

60. The anti-CD84 antibody of claim 59, wherein said therapeutic moiety is an anti-cancer moiety.

61. An isolated nucleic acid encoding an anti-CD84 antibody of any one of claim 1, 9, 20, 28, 36, 54, 55, 56, 57 or 58.

62. A cell comprising an anti-CD84 antibody of any one of claim 1, 9, 20, 28, 36, 54, 55, 56, 57 or 58, or a nucleic acid of claim 61.

63. A pharmaceutical composition comprising a therapeutically effective amount of an antibody of any one of claim 1, 9, 20, 28, 36, 54, 55, 56, 57 or 58 and a pharmaceutically acceptable excipient.

64. A method of forming an antibody capable of binding to CD84, said method comprises immunizing a mammal with a peptide comprising the sequence of SEQ ID NO: 111.

65. A method of treating cancer in a subject in need thereof, said method comprising administering to a subject a therapeutically effective amount of an anti-CD84 antibody of any one of claim 1, 9, 20, 28, 36, 54, 55, 56, 57 or 58 or a pharmaceutical composition of claim 63, thereby treating cancer in said subject.

66. The method of treating cancer of claim 65, wherein said cancer is lymphoma, myeloma, or leukemia.

67. The method of treating cancer of claim 65, wherein said cancer is acute myeloid leukemia (AML).


SEQUENCE LISTING (111 sequences)

SEQ ID NO: 1 - protein, 7 aa, synthetic construct
GFTFSDF

SEQ ID NO: 2 - protein, 7 aa, synthetic construct
RNKANDY

SEQ ID NO: 3 - protein, 9 aa, synthetic construct
DAVWEPFAY

SEQ ID NO: 4 - protein, 12 aa, synthetic construct
SANSSISSNSLH

SEQ ID NO: 5 - protein, 7 aa, synthetic construct
RTSNLAS

SEQ ID NO: 6 - protein, 9 aa, synthetic construct
QQGTSIPRT

SEQ ID NO: 7 - protein, 25 aa, synthetic construct
EVKLVESGGGLVQPGGSLRLSCATS

SEQ ID NO: 8 - protein, 19 aa, synthetic construct
YMDWVRQPPGKRLEWIATS

SEQ ID NO: 9 - protein, 42 aa, synthetic construct
TTEYSASVRGRFIVSRDTSQSILYLQMNALRAEDTAIYYCAR

SEQ ID NO: 10 - protein, 11 aa, synthetic construct
WGQGTLVTVSA

SEQ ID NO: 11 - protein, 23 aa, synthetic construct
EIVLTQSPTTMAASPGEKITVTC

SEQ ID NO: 12 - protein, 15 aa, synthetic construct
WYQQKPGFSPKLLIY

SEQ ID NO: 13 - protein, 32 aa, synthetic construct
GVPARFSGSGSGTSYFLTIGTMEAEDVATYYC

SEQ ID NO: 14 - protein, 10 aa, synthetic construct
FGGGTKLEIK

SEQ ID NO: 15 - protein, 120 aa, synthetic construct
EVKLVESGGGLVQPGGSLRLSCATSGFTFSDFYMDWVRQPPGKRLEWIATSRNKANDYTTEYSASVRGRFIVSRDTSQSILYLQMNALRAEDTAIYYCARDAVWEPFAYWGQGTLVTVSA

SEQ ID NO: 16 - protein, 108 aa, synthetic construct
EIVLTQSPTTMAASPGEKITVTCSANSSISSNSLHWYQQKPGFSPKLLIYRTSNLASGVPARFSGSGSGTSYFLTIGTMEAEDVATYYCQQGTSIPRTFGGGTKLEIK

SEQ ID NO: 17 - protein, 223 aa, synthetic construct
EVKLVESGGGLVQPGGSLRLSCATSGFTFSDFYMDWVRQPPGKRLEWIATSRNKANDYTTEYSASVRGRFIVSRDTSQSILYLQMNALRAEDTAIYYCARDAVWEPFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 18 - protein, 215 aa, synthetic construct
EIVLTQSPTTMAASPGEKITVTCSANSSISSNSLHWYQQKPGFSPKLLIYRTSNLASGVPARFSGSGSGTSYFLTIGTMEAEDVATYYCQQGTSIPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 9 aa, synthetic construct
GFSLNTSNM

SEQ ID NO: 20 - protein, 4 aa, synthetic construct
WWDD

SEQ ID NO: 21 - protein, 9 aa, synthetic construct
MRGDYTMDY

SEQ ID NO: 22 - protein, 15 aa, synthetic construct
RASQSVSTSLYSYMH

SEQ ID NO: 23 - protein, 7 aa, synthetic construct
YASDLES

SEQ ID NO: 24 - protein, 9 aa, synthetic construct
QHSWEIPYT

SEQ ID NO: 25 - protein, 25 aa, synthetic construct
QVTLKESGPGLLQPSQTLSLTCSFS

SEQ ID NO: 26 - protein, 19 aa, synthetic construct
GVGWIRQPSGKDLEWLAHI

SEQ ID NO: 27 - protein, 42 aa, synthetic construct
VKRYNPALKSRLTISKDTSNNQVFLKIASVDTADTATYYCAR

SEQ ID NO: 28 - protein, 11 aa, synthetic construct
WGQGTSVTVSS

SEQ ID NO: 29 - protein, 23 aa, synthetic construct
DIVLTQSPASLAVSLGQRATLSC

SEQ ID NO: 30 - protein, 15 aa, synthetic construct
WYQQKPGQPPKLLIK

SEQ ID NO: 31 - protein, 32 aa, synthetic construct
GVPARFSGSGSGTDFTLHIHPVEEEDTATYYC

SEQ ID NO: 32 - protein, 10 aa, synthetic construct
FGGGTKLEIK

SEQ ID NO: 33 - protein, 119 aa, synthetic construct
QVTLKESGPGLLQPSQTLSLTCSFSGFSLNTSNMGVGWIRQPSGKDLEWLAHIWWDDVKRYNPALKSRLTISKDTSNNQVFLKIASVDTADTATYYCARMRGDYTMDYWGQGTSVTVSS

SEQ ID NO: 34 - protein, 111 aa, synthetic construct
DIVLTQSPASLAVSLGQRATLSCRASQSVSTSLYSYMHWYQQKPGQPPKLLIKYASDLESGVPARFSGSGSGTDFTLHIHPVEEEDTATYYCQHSWEIPYTFGGGTKLEIK

SEQ ID NO: 35 - protein, 222 aa, synthetic construct
QVTLKESGPGLLQPSQTLSLTCSFSGFSLNTSNMGVGWIRQPSGKDLEWLAHIWWDDVKRYNPALKSRLTISKDTSNNQVFLKIASVDTADTATYYCARMRGDYTMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 36 - protein, 218 aa, synthetic construct
DIVLTQSPASLAVSLGQRATLSCRASQSVSTSLYSYMHWYQQKPGQPPKLLIKYASDLESGVPARFSGSGSGTDFTLHIHPVEEEDTATYYCQHSWEIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 220 aa, synthetic construct
EIQLVESGGGLVKPGGSLRLSCAASGYTFTTYSMHWVRQAPGKGLEWMGWIDTATGEPTLADDFKGRFTFSLDTSKNTAYLQMNSLKIEDTAVYFCVKSAYWYFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 38 - protein, 219 aa, synthetic construct
EVVMTQSPGTLSLSPGERATLSCRSSQSIVHINGNTYLEWYQQKPGQAPRLLIYKVSNRFSGIPDRFSGSGSGTDFTLTISRLEPEDFAVYYCFQGSHVPWTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 8 aa, synthetic construct
GYSITSDY

SEQ ID NO: 40 - protein, 5 aa, synthetic construct
SYSGT

SEQ ID NO: 41 - protein, 16 aa, synthetic construct
LHYYGSRYKKLYAMDY

SEQ ID NO: 42 - protein, 11 aa, synthetic construct
KASDNVGTYVS

SEQ ID NO: 43 - protein, 7 aa, synthetic construct
GASNRYT

SEQ ID NO: 44 - protein, 8 aa, synthetic construct
GQSYSYPT

SEQ ID NO: 45 - protein, 25 aa, synthetic construct
DVQLQESGPGLVKPSQSLSLTCTVT

SEQ ID NO: 46 - protein, 19 aa, synthetic construct
AWNWIRQFPGNKLEWMGYI

SEQ ID NO: 47 - protein, 41 aa, synthetic construct
TSYNPSLKSRISITRDTSKNQFFLQLNSVTTEDTATYYCAS

SEQ ID NO: 48 - protein, 11 aa, synthetic construct
WGQGTLATVSS

SEQ ID NO: 49 - protein, 23 aa, synthetic construct
NIVMTQSPKSMSMSVGERVTLSC

SEQ ID NO: 50 - protein, 15 aa, synthetic construct
WYQQKPEQSPKLLIY

SEQ ID NO: 51 - protein, 32 aa, synthetic construct
GVPDRFTGSGSATDFTLTISSVQAEDLADYHC

SEQ ID NO: 52 - protein, 10 aa, synthetic construct
FGGGTKLEIK

SEQ ID NO: 53 - protein, 125 aa, synthetic construct
DVQLQESGPGLVKPSQSLSLTCTVTGYSITSDYAWNWIRQFPGNKLEWMGYISYSGTTSYNPSLKSRISITRDTSKNQFFLQLNSVTTEDTATYYCASLHYYGSRYKKLYAMDYWGQGTLATVSS

SEQ ID NO: 54 - protein, 106 aa, synthetic construct
NIVMTQSPKSMSMSVGERVTLSCKASDNVGTYVSWYQQKPEQSPKLLIYGASNRYTGVPDRFTGSGSATDFTLTISSVQAEDLADYHCGQSYSYPTFGGGTKLEIK

SEQ ID NO: 55 - protein, 228 aa, synthetic construct
DVQLQESGPGLVKPSQSLSLTCTVTGYSITSDYAWNWIRQFPGNKLEWMGYISYSGTTSYNPSLKSRISITRDTSKNQFFLQLNSVTTEDTATYYCASLHYYGSRYKKLYAMDYWGQGTLATVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 56 - protein, 213 aa, synthetic construct
NIVMTQSPKSMSMSVGERVTLSCKASDNVGTYVSWYQQKPEQSPKLLIYGASNRYTGVPDRFTGSGSATDFTLTISSVQAEDLADYHCGQSYSYPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 7 aa, synthetic construct
GYTFTNY

SEQ ID NO: 58 - protein, 6 aa, synthetic construct
FPGGGY

SEQ ID NO: 59 - protein, 7 aa, synthetic construct
GNIAMDY

SEQ ID NO: 60 - protein, 10 aa, synthetic construct
SASSSVSYVH

SEQ ID NO: 61 - protein, 7 aa, synthetic construct
DTSKLAS

SEQ ID NO: 62 - protein, 9 aa, synthetic construct
QQWNNSPRT

SEQ ID NO: 63 - protein, 25 aa, synthetic construct
QVHLQQSGAELVRPGTSVKISCKAS

SEQ ID NO: 64 - protein, 19 aa, synthetic construct
WLGWVKQRPGHGLEWIGDI

SEQ ID NO: 65 - protein, 41 aa, synthetic construct
INYHEKFKGKATLTADTSSSTAYMQLSSLTSEDSAVYFCGY

SEQ ID NO: 66 - protein, 11 aa, synthetic construct
WGQGTSVTVSS

SEQ ID NO: 67 - protein, 23 aa, synthetic construct
QIILTQTPAIMSASPGEKVTMTC

SEQ ID NO: 68 - protein, 15 aa, synthetic construct
WYQQKSGTSPKRWIY

SEQ ID NO: 69 - protein, 32 aa, synthetic construct
GVPARFSGSGSGTSYSLTISTMEAEDAATYYC

SEQ ID NO: 70 - protein, 10 aa, synthetic construct
FGGGTKLEIK

SEQ ID NO: 71 - protein, 116 aa, synthetic construct
QVHLQQSGAELVRPGTSVKISCKASGYTFTNYWLGWVKQRPGHGLEWIGDIFPGGGYINYHEKFKGKATLTADTSSSTAYMQLSSLTSEDSAVYFCGYGNIAMDYWGQGTSVTVSS

SEQ ID NO: 72 - protein, 106 aa, synthetic construct
QIILTQTPAIMSASPGEKVTMTCSASSSVSYVHWYQQKSGTSPKRWIYDTSKLASGVPARFSGSGSGTSYSLTISTMEAEDAATYYCQQWNNSPRTFGGGTKLEIK

SEQ ID NO: 73 - protein, 219 aa, synthetic construct
QVHLQQSGAELVRPGTSVKISCKASGYTFTNYWLGWVKQRPGHGLEWIGDIFPGGGYINYHEKFKGKATLTADTSSSTAYMQLSSLTSEDSAVYFCGYGNIAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 74 - protein, 213 aa, synthetic construct
QIILTQTPAIMSASPGEKVTMTCSASSSVSYVHWYQQKSGTSPKRWIYDTSKLASGVPARFSGSGSGTSYSLTISTMEAEDAATYYCQQWNNSPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 7 aa, synthetic construct
GYTFTNY

SEQ ID NO: 76 - protein, 6 aa, synthetic construct
FPGGGY

SEQ ID NO: 77 - protein, 7 aa, synthetic construct
GNVAMDY

SEQ ID NO: 78 - protein, 10 aa, synthetic construct
SASSSVSYVH

SEQ ID NO: 79 - protein, 7 aa, synthetic construct
DTSKLAS

SEQ ID NO: 80 - protein, 9 aa, synthetic construct
QQWNNSPRT

SEQ ID NO: 81 - protein, 25 aa, synthetic construct
QVHLQQSGAELVRPGTSVKISCKAS

SEQ ID NO: 82 - protein, 19 aa, synthetic construct
WLGWVKQRPGHGLEWIGDI

SEQ ID NO: 83 - protein, 41 aa, synthetic construct
INYHEKFKGKATLTADTSSSTAYMQLSSLTSEDSAVYFCAY

SEQ ID NO: 84 - protein, 11 aa, synthetic construct
WGQGTSVTVSS

SEQ ID NO: 85 - protein, 23 aa, synthetic construct
QIILTQTPAIMSASPGEKVTVTC

SEQ ID NO: 86 - protein, 15 aa, synthetic construct
WYQQKSGTSPKRWIY

SEQ ID NO: 87 - protein, 32 aa, synthetic construct; identical to SEQ ID NO: 69

SEQ ID NO: 88 - protein, 10 aa, synthetic construct
FGGGTKLEIK

SEQ ID NO: 89 - protein, 116 aa, synthetic construct
QVHLQQSGAELVRPGTSVKISCKASGYTFTNYWLGWVKQRPGHGLEWIGDIFPGGGYINYHEKFKGKATLTADTSSSTAYMQLSSLTSEDSAVYFCAYGNVAMDYWGQGTSVTVSS

SEQ ID NO: 90 - protein, 106 aa, synthetic construct
QIILTQTPAIMSASPGEKVTVTCSASSSVSYVHWYQQKSGTSPKRWIYDTSKLASGVPARFSGSGSGTSYSLTISTMEAEDAATYYCQQWNNSPRTFGGGTKLEIK

SEQ ID NO: 91 - protein, 219 aa, synthetic construct
QVHLQQSGAELVRPGTSVKISCKASGYTFTNYWLGWVKQRPGHGLEWIGDIFPGGGYINYHEKFKGKATLTADTSSSTAYMQLSSLTSEDSAVYFCAYGNVAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 92 - protein, 213 aa, synthetic construct
QIILTQTPAIMSASPGEKVTVTCSASSSVSYVHWYQQKSGTSPKRWIYDTSKLASGVPARFSGSGSGTSYSLTISTMEAEDAATYYCQQWNNSPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 7 aa, synthetic construct
GYTFTTY

SEQ ID NO: 94 - protein, 6 aa, synthetic construct
DTATGE

SEQ ID NO: 95 - protein, 8 aa, synthetic construct
SAYWYFDV

SEQ ID NO: 96 - protein, 16 aa, synthetic construct
RSSQSIVHINGNTYLE

SEQ ID NO: 97 - protein, 7 aa, synthetic construct
KVSNRFS

SEQ ID NO: 98 - protein, 9 aa, synthetic construct
FQGSHVPWT

SEQ ID NO: 99 - protein, 25 aa, synthetic construct
QIQLVQSGPELKKPGETVKISCKAS

SEQ ID NO: 100 - protein, 19 aa, synthetic construct
SMHWVKQAPGKGLKWMGWI

SEQ ID NO: 101 - protein, 41 aa, synthetic construct
PTYADDFKGRFAFSLETSASTAYLQINNLNNDDTATYFCVK

SEQ ID NO: 102 - protein, 11 aa, synthetic construct
WGAGTTVTVSS

SEQ ID NO: 103 - protein, 23 aa, synthetic construct
DVLMTQTPLSLPVSLGDQASISC

SEQ ID NO: 104 - protein, 15 aa, synthetic construct
WYLQKPGQSPKLLIY

SEQ ID NO: 105 - protein, 32 aa, synthetic construct
GVPDRFSGSGSGTDFTLKISRVEAEDLGVYYC

SEQ ID NO: 106 - protein, 10 aa, synthetic construct
FGGGTKLEIK

SEQ ID NO: 107 - protein, 117 aa, synthetic construct
QIQLVQSGPELKKPGETVKISCKASGYTFTTYSMHWVKQAPGKGLKWMGWIDTATGEPTYADDFKGRFAFSLETSASTAYLQINNLNNDDTATYFCVKSAYWYFDVWGAGTTVTVSS

SEQ ID NO: 108 - protein, 112 aa, synthetic construct
DVLMTQTPLSLPVSLGDQASISCRSSQSIVHINGNTYLEWYLQKPGQSPKLLIYKVSNRFSGVPDRFSGSGSGTDFTLKISRVEAEDLGVYYCFQGSHVPWTFGGGTKLEIK

SEQ ID NO: 109 - protein, 220 aa, synthetic construct
QIQLVQSGPELKKPGETVKISCKASGYTFTTYSMHWVKQAPGKGLKWMGWIDTATGEPTYADDFKGRFAFSLETSASTAYLQINNLNNDDTATYFCVKSAYWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 110 - protein, 219 aa, synthetic construct
DVLMTQTPLSLPVSLGDQASISCRSSQSIVHINGNTYLEWYLQKPGQSPKLLIYKVSNRFSGVPDRFSGSGSGTDFTLKISRVEAEDLGVYYCFQGSHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 434 aa, synthetic construct
KDSEIFTVNGILGESVTFPVNIQEPRQVKIIAWTSKTSVAYVTPGDSETAPVVTVTHRNYYERIHALGPNYNLVISDLRMEDAGDYKADINTQADPYTTTKRYNLQIYRRLGKPKITQSLMASVNSTCNVTLTCSVEKEEKNVTYNWSPLGEEGNVLQIFQTPEDQELTYTCTAQNPVSNNSDSISARQLCADIAMGFRTHHTGGGGSKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK